SEQUENCE LISTING (44 sequences)

SEQ ID NO: 1 - protein, 404 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 2 - protein, 382 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 3 - protein, 381 aa, Homo sapiens
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 4 - protein, 339 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLG

SEQ ID NO: 5 - protein, 317 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLG

SEQ ID NO: 6 - protein, 316 aa, Homo sapiens
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLG

SEQ ID NO: 7 - protein, 94 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVR

SEQ ID NO: 8 - protein, 93 aa, Homo sapiens
QNITARIGEPLVLKCKGAPKKPPQRLEWKLNTGRTEAWKVLSPQGGGPWDSVARVLPNGSLFLPAVGIQDEGIFRCQAMNRNGKETKSNYRVR

SEQ ID NO: 9 - protein, 30 aa, Homo sapiens
AQNITARIGEPLVLKCKGAPKKPPQRLEWK

SEQ ID NO: 10 - protein, 29 aa, Homo sapiens
QNITARIGEPLVLKCKGAPKKPPQRLEWK

SEQ ID NO: 11 - protein, 98 aa, Homo sapiens
PEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALR

SEQ ID NO: 12 - protein, 91 aa, Homo sapiens
PRVWEPVPLEEVQLVVEPEGGAVAPGGTVTLTCEVPAQPSPQIHWMKDGVPLPLPPSPVLILPEIGPQDQGTYSCVATHSSHGPQESRAVS

SEQ ID NO: 13 - protein, 101 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGK

SEQ ID NO: 14 - protein, 100 aa, Homo sapiens
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGK

SEQ ID NO: 15 - protein, 114 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAG

SEQ ID NO: 16 - protein, 113 aa, Homo sapiens
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAG

SEQ ID NO: 17 - protein, 204 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQ

SEQ ID NO: 18 - protein, 203 aa, Homo sapiens
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQ

SEQ ID NO: 19 - protein, 229 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAP

SEQ ID NO: 20 - protein, 228 aa, Homo sapiens
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAP

SEQ ID NO: 21 - protein, 7 aa, Homo sapiens
VYQIPGK

SEQ ID NO: 22 - protein, 30 aa, Homo sapiens
TAPIQPRVWEPVPLEEVQLVVEPEGGAVAP

SEQ ID NO: 23 - protein, 20 aa, Homo sapiens
VYQIPGKPEIVDSASELTAG

SEQ ID NO: 24 - protein, 5 aa, Homo sapiens
TAPIQ

SEQ ID NO: 25 - DNA, 354 bp, Homo sapiens
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctac

SEQ ID NO: 26 - DNA, 369 bp, Homo sapiens
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaag

SEQ ID NO: 27 - DNA, 408 bp, Homo sapiens
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggt

SEQ ID NO: 28 - DNA, 690 bp, Homo sapiens
atggcagccggaacagcagttggagcctgggcgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagaggggagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgg

SEQ ID NO: 29 - DNA, 753 bp, Homo sapiens
atggcagccggaacagcagttggagcctgggcgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagaggggagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcct

SEQ ID NO: 30 - DNA, 1386 bp, Artificial Sequence
  Artificial = synthetic construct
atggcagccggaacagcagttggagcctgggcgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagaggggagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 31 - DNA, 1041 bp, Artificial Sequence
  Artificial = synthetic construct
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 32 - protein, 461 aa, Artificial Sequence
  Artificial = synthetic construct
MAAGTAVGAWALVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 439 aa, Artificial Sequence
  Artificial = synthetic construct
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 438 aa, Artificial Sequence
  Artificial = synthetic construct
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 346 aa, Artificial Sequence
  Artificial = synthetic construct
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 324 aa, Artificial Sequence
  Artificial = synthetic construct
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 323 aa, Artificial Sequence
  Artificial = synthetic construct
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 633 bp, Homo sapiens
ccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 40 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 663 bp, Homo sapiens
ccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 42 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 43 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 25 aa, Homo sapiens
ISIIEPGEEGPTAGSVGGSGLGTLA

(19) United States
(12) Patent Application Publication
(10) Pub. No.: US 2013/0064835 A1
(43) Pub. Date: Mar. 14, 2013

(54) RAGE REGULATES ROCK ACTIVITY IN CARDIOVASCULAR DISEASE

(71) Applicant: Ann Marie Schmidt, Franklin Lakes, NJ (US)

(72) Inventor: Ann Marie Schmidt, Franklin Lakes, NJ (US)

(21) Appl. No.: 13/500,885

(22) PCT Filed: Oct. 8, 2010
(86) PCT No.: PCT/US10/52069

§ 371 Date: Nov. 27, 2012

Related U.S. Application Data
(60) Provisional application No. 61/278,581, filed on Oct. 8, 2009.

(51) Int. Cl. A61K 39/395; A61P 11/00; A61K 38/17; A61K 31/517; A61K 31/4745; A61P 13/12; A61P 35/00

(52) U.S. Cl. 424/172.1; 514/266.23; 514/283; 514/15.4; 514/1.5; 514/19.3; 514/19.4

(57) ABSTRACT

A method is provided for treating a RAGE-related disorder in a subject afflicted therewith comprising administering to the subject a therapeutically effective amount of an antagonist of rho-associated protein kinase 1 (ROCK1) so as to thereby treat the RAGE-related disorder. A method is also provided for treating a ROCK1-related disorder in a subject afflicted therewith comprising administering to the subject therapeutically effective amount of antagonist of receptor for advanced glycation end products (RAGE) so as to thereby treat the ROCK-related disorder.

This application claims priority of U.S. Provisional Application No. 61/278,581, filed Oct. 8, 2009, the contents of which are hereby incorporated by reference into this application.

Throughout this application, various publications are referenced by citation or in parentheses by number. Full citations for the numbered references may be found at the end of the specification immediately preceding the claims. The disclosures of all of these publications in their entireties are hereby incorporated by reference into this application to more fully describe the state of the art to which this invention pertains.

The work disclosed herein was made with government support under grant no. HL60901 from the National Heart, Lung, and Blood Institute. Accordingly, the U.S. Government has certain rights in this invention.

### BACKGROUND

The multi-ligand Receptor for AGE (RAGE) contributes to atherosclerosis in ApoE null mice in both the non-diabetic and diabetic states. Previous studies using soluble RAGE, the extracellular ligand-binding domain of RAGE, or homozygous RAGE null mice showed that blockade or deletion of RAGE resulted in marked reduction in atherosclerotic lesion area and complexity compared to control animals (1-6). In parallel, significant down-regulation of inflammatory mediators and matrix metalloproteinases was evident in ApoE null mice aortas devoid of RAGE compared to those of RAGE-expressing ApoE null mice.

Although these findings suggested that RAGE modulated inflammatory gene expression in ApoE null mouse aorta, they did not reveal the pathways by which RAGE contributed to atherosclerosis.

### SUMMARY OF THE INVENTION

A method of treating a renal disease in a subject comprising administering to the subject an amount of an antagonist of receptor for advanced glycation end products (RAGE) effective to treat the renal disease in the subject.

A method of treating a disease involving apoptosis of cardiomyocytes in a subject comprising administering to the subject an amount of an antagonist of receptor for advanced glycation end products (RAGE) effective to treat the disease involving apoptosis of cardiomyocytes in the subject.

A method of treating a lung disease in a subject comprising administering to the subject an amount of an antagonist of receptor for advanced glycation end products (RAGE) effective to treat the lung disease in the subject.

A method of enhancing the efficacy of a chemotherapeutic agent in inducing apoptosis of a tumor cell in a subject comprising administering to the subject a chemotherapeutic agent and an amount of an antagonist of receptor for advanced glycation end products (RAGE) effective to enhance the efficacy of the chemotherapeutic agent in inducing apoptosis of the tumor cell the subject.

A method of treating a colorectal cancer, breast cancer, or pancreatic cancer in a subject comprising administering to the subject an amount of an antagonist of receptor for advanced glycation end products (RAGE) effective to treat the colorectal cancer, breast cancer, or pancreatic cancer in the subject.

A method of inhibiting metastasis of pancreatic cancer in a subject comprising administering to the subject an amount of an antagonist of receptor for advanced glycation end products (RAGE) effective to inhibit metastasis of the cancer in the subject.

A method of treating pancreatic inflammation in a subject comprising administering to the subject an amount of an antagonist of receptor for advanced glycation end products (RAGE) effective to treat pancreatic inflammation in the subject.

A method of treating cerebral vasospasm in a subject comprising administering to the subject an amount of an antagonist of receptor for advanced glycation end products (RAGE) effective to treat cerebral vasospasm in the subject.

A method of treating glaucoma in a subject comprising administering to the subject an amount of an antagonist of receptor for advanced glycation end products (RAGE) effective to treat glaucoma in the subject.

A method of treating tinnitus in a subject comprising administering to the subject an amount of an antagonist of receptor for advanced glycation end products (RAGE) effective to treat tinnitus in the subject.

A method of treating spinal cord injury in a subject comprising administering to the subject an amount of an antagonist of receptor for advanced glycation end products (RAGE) effective to treat spinal cord injury in the subject.

A method of treating a neurodegenerative disease in a subject comprising administering to the subject an amount of an antagonist of rho-associated protein kinase 1 (ROCK1) effective to treat the neurodegenerative disease in the subject.

A method of treating a diabetes-associated inflammatory disease in a subject comprising administering to the subject an amount of an antagonist of rho-associated protein kinase 1 (ROCK1) effective to treat the diabetes-associated inflammatory disease in the subject.

A method of treating a cardiovascular disease in a subject comprising administering to the subject an amount of an antagonist of rho-associated protein kinase 1 (ROCK1) effective to treat the cardiovascular disease in the subject.

A method of treating a vascular disease in a subject comprising administering to the subject an amount of an antagonist of rho-associated protein kinase 1 (ROCK1) effective to treat the vascular disease in the subject.

A method of treating a receptor for advanced glycation end products (RAGE)-associated inflammatory disease in a subject comprising administering to the subject an amount of an antagonist of rho-associated protein kinase 1 (ROCK1) effective to treat the RAGE-associated chronic inflammatory disease in the subject.

A method of treating a renal cell carcinoma, prostate cancer, biliary cancer, or lung cancer in a subject comprising administering to the subject an amount of an antagonist of rho-associated protein kinase 1 (ROCK1) effective to treat the renal cell carcinoma, prostate cancer, biliary cancer, breast cancer or lung cancer in the subject.

A method of promoting survival of a liver in a subject subsequent to a partial hepatectomy in the subject comprising administering to the subject before, after or during the partial hepatectomy an amount of an antagonist of rho-associated protein kinase 1 (ROCK1) effective to promote survival of the liver in the subject.

A method of treating metabolic syndrome in a subject comprising administering to the subject an amount of an antagonist of rho-associated protein kinase 1 (ROCK1) effective to treat metabolic syndrome in the subject.

A method of treating obesity in a subject comprising administering to the subject an amount of an antagonist of rho-associated protein kinase 1 (ROCK1) effective to treat obesity in the subject.

A method of treating periodontal disease in a subject comprising administering to the subject an amount of an antagonist of rho-associated protein kinase 1 (ROCK1) effective to treat the periodontal disease in the subject.

A method for treating hyperglycemia in a subject comprising administering to the subject an antagonist of an antagonist of rho-associated protein kinase 1 (ROCK1) in an amount effective to treat hyperglycemia in the subject.

A method for reducing levels of insulin in blood in a subject comprising administering to the subject an antagonist of rho-associated protein kinase 1 (ROCK1) in an amount effective to reduce insulin levels in blood in the subject.

A method for reducing levels of blood cholesterol in a subject comprising administering to the subject an antagonist of rho-associated protein kinase 1 (ROCK1) in an amount effective to reduce blood cholesterol levels in the subject.

A method for reducing levels of triglycerides in a subject comprising administering to the subject an antagonist of rho-associated protein kinase 1 (ROCK1) in an amount effective to reduce triglyceride levels in the subject.

A method for reducing levels of leptins in a subject comprising administering to the subject an antagonist of rho-associated protein kinase 1 (ROCK1) in an amount effective to reduce leptin levels in the subject.

A method for treating a subject with a condition associated with interaction of an amyloid-beta peptide with RAGE on a cell.

A method of treating a symptom of diabetes in a diabetic subject which comprises administering to the subject an antagonist of rho-associated protein kinase 1 (ROCK1) in an amount effective treat the symptom of diabetes in the subject.

A method of alleviating a RAGE-associated inflammation in a subject which comprises administering to the subject an antagonist of rho-associated protein kinase 1 (ROCK1) in an amount effective treat the RAGE-associated inflammation in the subject.

A method of inhibiting metastasis of a non-pancreatic cancer in a subject comprising administering to the subject an antagonist of rho-associated protein kinase 1 (ROCK1) effective to inhibit metastasis of the non-pancreatic cancer in the subject.

A method of inhibiting new tissue growth in blood vessels in a subject, wherein the subject has experienced blood vessel injury, which comprises administering to the subject an antagonist of rho-associated protein kinase 1 (ROCK1) in an amount effective so as to inhibit new tissue growth in the subject's blood vessels.

A method of inhibiting neointimal formation in blood vessels in a subject, wherein the subject has experienced blood vessel injury, which comprises administering to the subject an antagonist of rho-associated protein kinase 1 (ROCK1) in an amount effective so as to inhibit neointimal formation in blood vessels in the subject's blood vessels.

A method of inhibiting the onset of glomerulosclerosis, proteinuria, or albunuria in a subject comprising administering to the subject a prophylactically effective amount of an antagonist of rho-associated protein kinase 1 (ROCK1) so as to thereby inhibit the onset of glomerulosclerosis, proteinuria, or albunuria in the subject.

A method for treating a RAGE-related disorder in a subject afflicted therewith comprising administering to the subject a therapeutically effective amount of an antagonist of rho-associated protein kinase 1 (ROCK1) so as to thereby treat the RAGE-related disorder.

A method for treating a ROCK1-related disorder in a subject afflicted therewith comprising administering to the subject a therapeutically effective amount of antagonist of receptor for advanced glycation end products (RAGE) so as to thereby treat the ROCK-related disorder.

### BRIEF DESCRIPTION OF THE FIGURES

FIG. 1. Tgf-β KEGG Pathway analysis: effect of diabetes in ApoE null mice. Tgf-β KEGG Pathway, gene symbols that are colored reflect genes that are statistically significantly differentially expressed in ApoE null mice with diabetes relative to non-diabetic ApoE null mice. Up-regulated genes are shown in dark gray, and down-regulated genes are shown in black. Numbers indicate perturbation factors (which may be different in magnitude and even in sign than fold-changes. KEGG Pathways often represent several different and related proteins by a single protein [for example, Tgf-β1, Tgf-β2, and Tgf-β3 are all represented as Tgf-β]). In such a case, the perturbation factor for the product of the gene with non-zero fold change is given in the figure.

FIG. 2. Tgf-β KEGG Pathway analysis: effect of deletion of RAGE in diabetic ApoE null mice. Tgf-β KEGG Pathway, gene symbols that are colored reflect genes that are statistically significantly differentially expressed in diabetic ApoE null/RAGE null mice relative to diabetic ApoE null mice. Down-regulated genes are shown in black. Numbers indicate perturbation factors as in FIG. 1.

FIG. 3. Regulation of Thbs1, TGFβ-2 and ROCK1 protein in ApoE null mouse aorta. In order to validate the mRNA transcript findings on the above candidate genes, total aorta tissue was lysed and subjected to Western blotting as described above using primary antibodies for detection of Thbs1 (A), Tgf-β2 (B) and ROCK1 (C). After probing with the primary antibody, membranes were stripped and re-probed with antibodies to detect GAPDH. In each case, lane 1 represents non-diabetic ApoE null; lane 2 represents diabetic ApoE null; lane 3 represents non-diabetic ApoE null/RAGE null and lane 4 represents diabetic ApoE null/RAGE null. Statistical analyses for these data are illustrated in Table 6.

FIG. 4. Localization of RAGE, Thbs1, Tgf-β2 and ROCK1 antigens in the aortas of non-diabetic and diabetic ApoE null and ApoE null/RAGE null mice. Confocal microscopy was performed on aorta tissue and subjected to immunostaining for detection of the following antigens: RAGE antigen. (A). Left column reveals staining with a RAGE specific antibody. Middle column reveals staining with monoclonal mouse smooth muscle actin antibody specific to smooth muscle cell α-actin. Right column reveals the merge of left and right column images. (B). Left column reveals staining with RAGE-specific antibody. Middle column reveals staining with antibody specific for CD31/PECAM1. Right column reveals the merge of left and right column images. Single black square reveals staining with nonimmune IgG control. Thbs1 antigen. (C). Left column reveals staining with a Thbs1 specific antibody. Middle column reveals staining with RAGE specific antibody. Right column reveals the merge of left and right column images.(D) Left column reveals staining with Thbs1-specific antibody. Middle column reveals staining with smooth muscle cell specific antibody. Right column reveals the merge of left and right column images. (E). Left column reveals staining with Thbs1 specific antibody. Middle column reveals staining with CD31/PECAM specific antibody. Right column reveals merge of left and right column images. Single black square reveals staining with nonimmune IgG control. Tgf-β2 antigen. (F). Left column reveals staining with a Tgf-β2 specific antibody. Middle column reveals staining with RAGE specific antibody. Right column reveals the merge of left and right column images. (G). Left column reveals staining with Tgf-β2 specific antibody. Middle column reveals staining with smooth muscle cell specific antibody. Right column reveals the merge of left and right column images. (H). Left column reveals staining with Tgf-β2 specific antibody. Middle column reveals staining with CD31/PECAM1 specific antibody. Right column reveals merge of left and right column images. Single black square reveals staining with nonimmune IgG control. ROCK1 antigen. (I). Left column reveals staining with a ROCK1 specific antibody. Middle column reveals staining with RAGE specific antibody. Right column reveals the merge of left and right column images. (J). Left column reveals staining with ROCK1 specific antibody. Middle column reveals staining with smooth muscle cell specific antibody. Right column reveals the merge of left and right column images. (K). Left column reveals staining with ROCK1 specific antibody. Middle column reveals staining with CD31/PECAM1 specific antibody. Right column reveals merge of left and right column images. Single black square reveals staining with nonimmune IgG control. Original magnifications: ×200.

FIG. 5. ROCK1 activation in ApoE null aorta and primary SMCs: effect of RAGE. Aortas were retrieved from the indicated mice at age 9 weeks (a) or primary murine aortic SMCs were treated with S100b for the indicated times (b). Lysates were prepared and ROCK1 activity determined. Statistical considerations are indicated in the figure.

FIG. 6. Venn diagram depicting the effect of diabetes and RAGE deletion in ApoE null mouse aorta. The Venn diagram shows the intersection of comparison 1, diabetic ApoE null relative to non-diabetic ApoE null, with comparison 4, diabetic ApoE null/RAGE null relative to diabetic ApoE null. Although there are 53 genes which are statistically significantly differentially expressed in diabetic ApoE null relative to the non-diabetic ApoE null state, and 216 genes which are statistically significantly differentially expressed in diabetic ApoE null/RAGE null relative to diabetic ApoE null, only 15 of these genes are statistically significantly differentially expressed in both comparisons. Note that there is very little overlap of the genes which are differentially expressed both in the onset of diabetes in apoE null mice and in the effect of RAGE deletion in diabetic ApoE null mice.

FIG. 7. Deletion of RAGE suppresses diabetes-accelerated atherosclerosis. 7A: Male ApoE null (N=8) and Apo E null/RAGE null mice (N=7) were rendered diabetic with streptozotocin at age 6 weeks. Mice were sacrificed at age 14 weeks and aortas were retrieved. Mean atherosclerotic lesion area at the aortic sinus is reported; statistical considerations are indicated in the text. 7B-7C: At age 24 weeks, an age at which significant lesions would have formed in RAGE-expressing ApoE null mice, a significant increase in % macrophages/lesion area and % SMCs/lesion area was observed in diabetic ApoE null vs. non-diabetic ApoE null mice.

FIG. 8. Studies in primary SMCs retrieved from RAGE-expressing or RAGE-deficient mouse aortas. 8A and 8B: show that incubation of wild-type SMCs with RAGE ligand S1000 resulted in significantly increased proliferation and migration, but S100B failed to stimulate proliferation and migration in RAGE-deficient SMCs. 8C and 8D: pre-treatment of wild-type SMCs with anti-Tgf-β2 antibody resulted in a significant decrease in proliferation and migration compared to treatment with an IgG control. 8E and 8F: treatment of SMCs with S100B in the presence of ROCK inhibitors Y27632 or fasudil significantly reduced S100B-stimulated proliferation and migration.

FIG. 9. Proposed mechanism by which diabetes and RAGE contribute to atherosclerosis in ApoE null mice. Based on in-depth analysis of microarray findings, the mechanisms by which diabetes accelerates atherosclerosis in ApoE null mice (A) and by which RAGE accelerates atherosclerosis in diabetic ApoE null mice (B) are considered. In both cases, the left column represents the pathway, and the right column represents the observed change in concentration of mRNA and protein and inferred change in activation of proteins and processes. Numbers accompanying each molecular step are Pathway Express perturbation factors. Note that Tgf-□R appears in two steps for the sake of reliability, but only has one perturbation factor, as any other protein in the pathway.

FIG. 10. Key to left column symbols of FIG. 9.

FIG. 11. Key to right column symbols of FIG. 9.

### DETAILED DESCRIPTION OF THE INVENTION

The multi-ligand Receptor for AGE (RAGE) (SEQ ID NO:1) contributes to atherosclerosis in apolipoprotein (ApoE) null mice.

As used herein, an antagonist of rho-associated protein kinase 1 (ROCK1) is a chemical substance, for example a small molecule or an antibody, which reverses, reduces or blocks the physiological effect induced by ROCK1 acting as an agonist.

A “RAGE” antagonist is a chemical substance, for example a small molecule or an antibody, which reverses, reduces or blocks the physiological effect induced by RAGE acting as an agonist.

A small molecule as used herein is an organic molecule or organic-based molecule having a molecular weight of less than 200 Daltons. In an embodiment, the small molecules of the present invention are those having a molecular weight of less than 160 Daltons. In an embodiment, the small molecule is an organic small molecule.

Non-limiting examples of RAGE fusion proteins that can be used as RAGE antagonists in the present invention are described, for example, in the following publications: PCT International Application Publication No. WO/2008/100470; PCT International Application Publication No. WO/2004/016229; PCT International Application Publication No. WO 2006/017647 A1; PCT International Application Publication No. WO 2006/017643 A1; U.S. Patent Application Publication No. US 2006/140933; U.S. Patent Application Publication No. US 2006/078562; U.S. Patent Application No. US 2006/0057679; U.S. Patent Application Publication No. 2006/0030527; PCT International Application Publication No. WO/2007/094926; U.S. Patent Application Publication No. US 2006-0078562 A1; and U.S. Pat. No. 7,470,521, all of which are hereby incorporated by reference in their entireties. RAGE fusion proteins which can be used as the RAGE antagonists recited in the present invention include those comprising soluble RAGE (sRAGE) (SEQ ID NO:4) or a derivative thereof, for example a polypeptide being identical to SEQ ID NO:4 except for a glycine as as residue no. 1 instead of a methionine, those comprising the V-domain of RAGE (SEQ ID NO:7; SEQ ID NO:8) the RAGE ligand binding site (SEQ ID NO:9; SEQ ID NO:10), and those comprising RAGE (SEQ ID NO:1) or a portion thereof but without the first 19, 20, 21, 22 or 23 (leader sequence) amino acids, for example SEQ ID NOs:5, 6, 7, 8, 11, 12, 13, 14, 15, 16, 17, or 18. In addition, fusion proteins comprising fragments of these sequences may be used. The second polypeptide of the RAGE fusion proteins can comprise a non-RAGE polypeptide such as an immunoglobulin derived polypeptide, e.g. a human IgG-derived polypeptide. The second polypeptide of the RAGE fusion proteins can comprise a heavy chain fragment such as an Fc fragment, for example a heavy chain hinge polypeptide. In an embodiment the second polypeptide of the RAGE fusion protein is a C_{H}2 and/or C_{H}3 domain of an immunoglobulin (for example see SEQ ID NOs 38 and 40). In an embodiment the second polypeptide of the RAGE fusion protein is a RAGE polypeptide as described above (e.g. sRAGE) linked to a polypeptide comprising a C_{H}2 domain of an immunoglobulin. In an embodiment the second polypeptide can comprise an interdomain linker derived from RAGE. In an embodiment the C_{H}2 domain comprises SEQ ID NO:42. RAGE fusion proteins that may be employed as RAGE antagonists in the current invention are also described in PCT International Application Publication No. WO 2006/017643 which is hereby incorporated by reference in its entirety. In an embodiment the RAGE fusion protein comprises the sequence set forth in SEQ ID NO:30 or SEQ ID NO:31. In an embodiment the RAGE fusion protein comprises the sequence set forth in one of SEQ ID NOs:32-37.

Non-limiting examples of other RAGE antagonists are described, for example, in the following publications: U.S. Patent Application Publication No. US 2008/119512; U.S. Pat. No. 7,361,678; PCT International Application Publication No. WO/2003/075921; PCT International Application Publication No. WO 2007/089616; PCT International Application Publication No. WO 2007/076200; PCT International Application Publication No. WO 2007/0286858; PCT International Application Publication No. WO/2008/153957; PCT International Application Publication No. WO/2008/123914; PCT International Application Publication No. WO/2007/130302; U.S. Pat. No. 7,361,678; U.S. Pat. No. 7,423,177; U.S. Pat. No. 7,087,632; U.S. Pat. No. 7,361,678; U.S. Pat. No. 7,067,554; U.S. Pat. No. 6,613,801; U.S. Pat. No. 5,864,018, all of which are hereby incorporated by reference in their entireties.

Other examples of RAGE antagonists that can be employed in the methods described herein are anti-RAGE antibodies (e.g. see Lutterloh, E., Expert Opinion on Pharmacotherapy, June 2007, Vol. 8, No. 9, Pages 1193-1196 and Flyvbjerg at al. Diabetes January 2004 vol. 53 no. 1 166-172; both of which are hereby incorporated by reference in their entirety). In an embodiment the anti-RAGE antibody is a monoclonal antibody. Examples are those disclosed in Lutterloh et al., Crit. Care 11(6):R122 (2007), ccforum.com/content/11/6/R122), which is hereby incorporated by reference in its entirety.

Other examples of RAGE antagonists are TransTech Pharma TTP448 and TransTech Pharma TTP4000 (TransTech, North Carolina, USA). In an embodiment, the RAGE antagonist is a small molecule. In one embodiment, the small molecule is a compound having the structure:

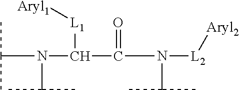

wherein L1 is a C1-C4 alkyl group and L2 is a direct bond, and Aryl₁ and Aryl₂ are aryl, wherein each of Aryl₁ and Aryl₂ are substituted by at least one lipophilic group selected from the group consisting of
a) —Y—C1-6 alkyl;

### b) —Y-aryl;

c) —Y—C-1-6 alkylaryl;
d) —Y—C1-6-alkyl-NR7R8;
e) —Y—C1-6-alkyl-W—R20;
wherein
   Y and W are, independently selected from the group consisting of —CH2-, —O—, —N(H), —S—, SO2-, —CON(H)—, —NHC(O)—, —NHCON(H)—, —NHSOa2-, —SO2(H)—, —C(O)—O—, —NHSO2NH—, —O—CO—,

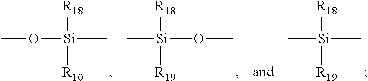

and
f) halogen, hydroxyl, cyano, carbamoyl, and carboxyl;
wherein
R18 and R19 are independently selected from the group consisting of aryl, C1-C6 alkyl, C1-C6 alkylaryl, C1-C6 alkoxy, and C1-C6 alkoxyaryl;
R20 is selected from the group consisting of aryl, C1-C6 alkyl, and C1-C6 alkylaryl;
R7, R8, R9 and R10 are independently selected from the group consisting of hydrogen, aryl, C1-C6 alkyl, and C1-C6 alkylaryl; and wherein R7 and R8 may be taken together to form a ring having the formula —(CH₂)ₘ—X—(CH₂)n- bonded to the nitrogen atom to which R7 and R8 are attached, wherein m and n are, independently, 1, 2, 3, or 4; X is selected from the group consisting of —CH2-, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—,

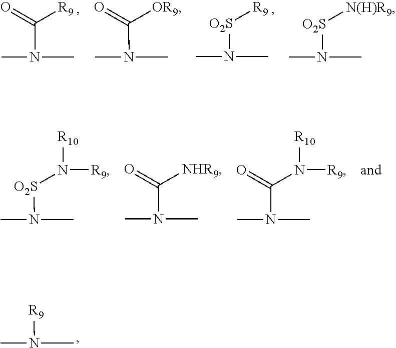

or a pharmaceutically acceptable salt thereof, wherein at least one of Aryl₁ and Aryl₂ is substituted with a lipophilic group of the formula —Y—C1-6-alkyl-NR₇R₈.

In one embodiment, the small molecule is a compound having the structure:

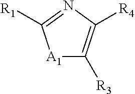

wherein
   R₁ is -hydrogen, -alkyl, -alkenyl, or -alkynyl, Alis —N(R₂)—;
   wherein
   R₂ is -phenyl,

### R₃ is

a) -hydrogen,
b) -halogen,
c) -hydroxyl,
d) -cyano,
e) -carbamoyl,
f) -carboxyl,
g) -aryl,
h) -cycloalkyl,
i) -alkyl,
j) -alkenyl,
k) -alkynyl,
l) -alkylene-aryl,
m) -alkylene-cycloalkyl,
n) -fused cycloalkylaryl,
o) -alkylene-fused cycloalkylaryl,

### p) —C(O)—O-alkyl,

### q) —C(O)—O-alkylene-aryl,

### r) —C(O)—NH-alkyl,

### s) —C(O)—NH-alkylene-aryl,

t) —SO₂-alkyl,
u) —SO₂-alkylene-aryl,
v) —SO₂-aryl,

### w) —SO₂—NH-alkyl,

### x) —SO₂NH-alkylene-aryl,

### y) —C(O)-alkyl,

### z) —C(O)-alkylene-aryl,

### aa) -G4-G5-G6-R7,

bb) —Y1-alkyl,
cc) —Y1-aryl,
dd) —Y1-alkylene-aryl,
ee) —Y1-alkylene-NR₉R₁₀, or
ff) —Y1-alkylene-W1-R₁₁,
wherein
G4 and G6 are independently selected from the group consisting of: alkylene, alkenylene, alkynylene, cycloalkylene, arylene, -alkylene-aryl, -alkenylene-aryl, -alkenylene-heteroaryl, and a direct bond;
G5 is —O—, —S—, —N(R₈)—, —S(O)—, —S(O)2-, —C(O)—, —O—C(O)—, —C(O)—O—, —C(O)N(R₈)—, —N(R₈)C(O)—, —S(O)₂N(R₈)—, N(R₈)S(O)₂—, —O-alkylene-C(O)—, —(O)C-alkylene-O—, —O-alkylene-, -alkylene-O—, alkylene, alkenylene, alkynylene, cycloalkylene, arylene, fused cycloalkylarylene, or a direct bond, wherein R₈ is -hydrogen, -aryl, -alkyl, -alkylene-aryl, or -alkylene-O-aryl;
wherein
R₇ is -hydrogen, -aryl, -cycloalkyl, -alkyl, -alkenyl, -alkynyl, -alkylene-aryl, -alkylene-cycloalkyl, -fused cycloalkylaryl, or -alkylene-fused cycloalkylaryl;
   Y1 and W1 are independently selected from the group consisting of —CH₂—, —O—, —N(H), —S—, —SO₂—, —CON(H)—, —NHC(O)—, —NHCON(H)—, —NHSO₂—, —SO₂N(H)—, —C(O)—O—, —NHSO₂NH—, —O—CO—,

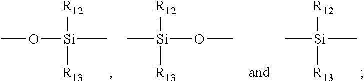

wherein
R₁₂ and R₁₃ are independently selected from the group consisting of: -aryl, -alkyl, -alkylene-aryl, -alkoxy, and -alkylene-O-aryl; and R₉, R₁₀, and R₁₁ are independently selected from the group consisting of: -aryl, -alkyl, and -alkylene-aryl;

### R4 is

a) -phenyl,
b) -phenylene-G5-G6-R7,
c) -phenylene-alkylene-G5-G6-R7, or
d) -phenylene-alkenylene-G5-G6-R7,
wherein
G6 is alkylene, alkenylene, alkynylene, cycloalkylene, heterocyclylene, arylene, heteroarylene, -alkylene-aryl, -alkylene-heteroaryl, -alkenylene-aryl, -alkenylene-heteroaryl, or a direct bond;
G5 is —O—, —S—, —N(R₈)—, —S(O)—, —S(O)₂—, —C(O)—, —O—C(O)—, —C(O)—O—, —C(O)N(R₈)—, N(R₈)C(O)—, —S(O)₂N(R₈)—, N(R₈)S(O)₂—, —O-alkylene-C(O)—, —(O)C-alkylene-O—, —O-alkylene-, -alkylene-O—, alkylene, alkenylene, alkynylene, cycloalkylene, heterocyclylene, arylene, heteroarylene, fused cycloalkylarylene, fused cycloalkylheteroarylene, fused heterocyclylarylene, fused heterocyclylheteroarylene, or a direct bond,
wherein
R₈ is -hydrogen, -aryl, -alkyl, -alkylene-aryl, or -alkylene-O-aryl; R7 is hydrogen, aryl, heteroaryl, cycloalkyl, heterocyclyl, alkyl, alkenyl, alkynyl, -alkylene-aryl, -alkylene-heteroaryl, -alkylene-heterocyclyl, -alkylene-cycloalkyl, fused cycloalkylaryl, fused cycloalkylheteroaryl, fused heterocyclylaryl, fused heterocyclylheteroaryl, alkylene-fused cycloalkylaryl, -alkylene-fused cycloalkylheteroaryl, -alkylene-fused heterocyclylaryl, or -alkylene-fused heterocyclylheteroaryl;
wherein
the aryl and/or alkyl group(s) in R₃, R₇, R₈, R₉, R₁₀, R₁₁, R₁₂, and R₁₃, may be optionally substituted 1-4 times with a substituent group, wherein said substituent group(s) are independently selected from the group consisting of:

### a) —H,

b) -halogen,
c) -hydroxyl,
d) -cyano,
e) -carbamoyl,
f) -carboxyl,
g) —Y2-alkyl,
h) —Y2-aryl,
i) —Y2-alkylene-aryl,
j) —Y2-alkylene-W2-R₁₈,

### k) —Y3-Y4-NR23R24,

### l) —Y3-Y4-NH—C(═NR₂₅)NR₂₃R₂₄, and

### m) —Y3-Y4-C(═NR₂₅)NR₂₃R₂₄,

wherein
Y2 and W2 are independently selected from the group consisting of —CH₂—, —N(H), —S—, SO₂—, —CON(H)—, —NHC(O)—, —NHCON(H)—, —NHSO₂—, —SO₂N(H)—, —C(O)—O—, —NHSO₂NH—, —O—S(O)₂—, —O—CO—,

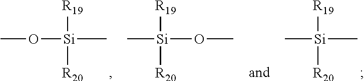

wherein R₁₉ and R₂₀ are independently selected from the group consisting of: -hydrogen, -aryl, -alkyl, -alkylene-aryl, -alkoxy, and -alkylene-O-aryl; R₁₈ is -aryl, -alkyl, -alkylene-aryl, or -alkylene-O-aryl;
Y3 is selected from the group consisting of a direct bond, —CH2-, —O—, —N(H), —S—, —SO₂—, —C(O)—, —CON(H)—, —NHC(O)—, —NHCON(H)—, —NHSO₂—, —SO₂N(H)—, —C(O)—O—, —NHSO₂NH—, —O—CO—,

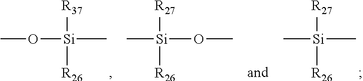

wherein R₂₇ and R₂₆ are independently selected from the group consisting of: -aryl, -alkyl, -alkylene-aryl, -alkoxy, and -alkyl-O-aryl;

### Y4 is

a) -alkylene,
b) -alkenylene,
c) -alkynylene,
d) -arylene,
e) -cycloalkylene,
f) -alkylene-arylene,
g) -alkylene-cycloalkylene,
h) -arylene-alkylene,
i) -cycloalkylene-alkylene,

### j) —O—,

### k) —S—,

### l) —S(O)₂—, or

### m) —S(O)—,

wherein said alkylene groups may optionally contain one or more O, S, S(O), or SO₂ atoms;
and R₂₃, R₂₄, and R₂₅ are independently selected from the group consisting of: -hydrogen, -aryl, -alkyl, -alkylene-aryl, and -alkylene-O-aryl,
and
wherein
R2 may be optionally substituted 1-4 times with a substituent group, wherein said substituent group(s) are independently selected from the group consisting of:

### a) —H,

b) -halogen,
c) -hydroxyl,
d) -cyano,
e) -carbamoyl,
f) -carboxyl,
g) —Y2-alkyl,
h) —Y2-aryl,
i) —Y2-heteroaryl,
j) —Y2-alkylene-heteroaryl-aryl,
k) —Y2-alkylene-aryl,
l) —Y2-alkylene-W2-R₁₈,

### m) —Y3-Y4-NR₂₃R₂₄,

### n) —Y3-Y4-NH—C(═NR₂₅)NR₂₃R₂₄,

### o) —Y3-Y4—C(═NR₂₅)NR₂₃R₂₄, and

### p) —Y3-Y4-Y5-A2,

wherein
Y2 and W2 are independently selected from the group consisting of —CH₂—, —O—, —N(H), —S—, SO₂—, —CON(H)—, —NHC(O)—, —NHCON(H)—, —NHSO₂—, —SO2N(H)—, —C(O)—O—, —NHSO₂NH—, —O—S(O)₂—, —O—CO—,

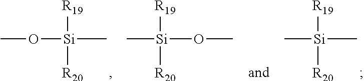

wherein R₁₉ and R₂₀ are independently selected from the group consisting of: -hydrogen, -aryl, -alkyl, -alkylene-aryl, alkoxy, and -alkylene-O-aryl;
R18 is -aryl, -alkyl, -alkylene-aryl, -alkylene-heteroaryl, or -alkylene-O-aryl,
Y3 and Y5 are independently selected from the group consisting of a direct bond, —CH₂—, —O—, —N(H), —S—, SO₂—, —C(O)—, —CON(H)—, —NHC(O)—, —NHCON(H)—, —NHSO₂—, —SO₂N(H)—, —C(O)—O—, —NHSO2NH—, —O—, CO—,

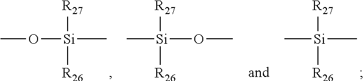

wherein R₂₇ and R₂₆ are independently selected from the group consisting of: -aryl, -alkyl, -alkylene-aryl, -alkoxy, and -alkyl-O-aryl;

### Y4 is

a) -alkylene,
b) -alkenylene,
c) -alkynylene,
d) -arylene,
e) -heteroarylene,
f) -cycloalkylene,
g) -heterocyclylene,
h) -alkylene-arylene,
i) -alkylene-heteroarylene,
j) -alkylene-cycloalkylene,
k) -alkylene-heterocyclylene,
l) -arylene-alkylene,
m) -heteroarylene-alkylene,
n) -cycloalkylene-alkylene,
o) -heterocyclylene-alkylene,

### p) —O—,

### q) —S—,

### r) —S(O)₂—, or

### s) —S(O)—,

wherein said alkylene groups may optionally contain one or more O, S, S(O), or SO₂ atoms;

### A2 is

a) heterocyclyl, fused arylheterocyclyl, or fused heteroarylheterocyclyl, containing at least one basic nitrogen atom, or
b) -imidazolyl, and
R₂₃, R₂₄, and R₂₅ are independently selected from the group consisting of: -hydrogen, -aryl, -heteroaryl, -alkylene-heteroaryl, -alkyl, -alkylene-aryl, -alkylene-O-aryl, and -alkylene-O-heteroaryl; and R₂₃ and R₂₄ may be taken together to form a five-membered ring having the formula —(CH₂)s-X3-(CH₂)t- bonded to the nitrogen atom to which R₂₃ and R₂₄ are attached
wherein
s and t are, independently, 1, 2, 3, or 4;
X3 is a direct bond, —CH₂—, —O—, —S—, —S(O)₂—, —C(O)—, —CON(H)—, —NHC(O)—, —NHCON(H)—, —NHSO₂—, —SO₂N(H)—, —C(O)—O—, —O—C(O)—, —NHSO2NH—,

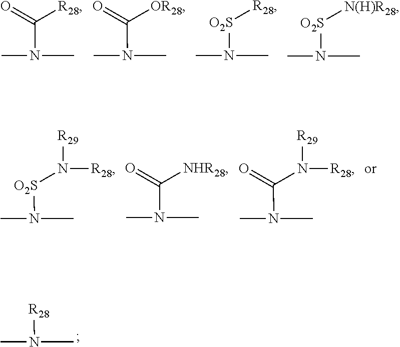

wherein R₂₈ and R₂₉ are independently selected from the group consisting of: -hydrogen, -aryl, -heteroaryl, -alkyl, -alkylene-aryl, and -alkylene-heteroaryl;
wherein the alkyl and/or aryl groups in the optional substituents
g) —Y2-alkyl,
h) —Y2-aryl,
i) —Y2-heteroaryl,
j) —Y2-alkylene-heteroaryl,
k) —Y2-alkylene-aryl,
l) —Y2-alkylene-W2-R₁₈,

### m) —Y3-Y4-NR₂₃R₂₄,

### n) —Y3-Y4-NH—C(═NR₂₅)NR₂₃R₂₄,

### o) —Y3-Y4-C(═NR₂₅)NR₂₃R₂₄, and

p)—Y3-Y4-Y5-A2,
of R2 may be optionally substituted 1-4 times with a substituent independently selected from the group consisting of:
a) halogen,
b) perhaloalkyl,
c) alkyl,
d) cyano,
e) alkyloxy,
f) aryl, and
g) aryloxy, and
wherein the aryl and/or alkyl group(s) in R4 may be optionally substituted 1-4 times with a substituent group, wherein said substituent group(s) are independently selected from the group consisting of:

### a) —H,

b) -halogen,
c) -hydroxyl,
d) -cyano,
e) -carbamoyl,
f) -carboxyl,
g) —Y2-alkyl,
h) —Y2-aryl,
i) —Y2-heteroaryl,
j) —Y2-alkylene-heteroaryl-aryl,
k) —Y2-alkylene-aryl,
l) —Y2-alkylene-W2-R₁₈,

### m) —Y3-Y4-NR₂₃R₂₄,

### n) —Y3-Y4-NH—C(═NR₂₅)NR₂₃R₂₄,

### o) —Y3-Y4-C(═NR₂₅)NR₂₃R₂₄, and

### p) —Y3-Y4-Y5-A2,

wherein
Y2 and W2 are independently selected from the group consisting of —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—S(O)₂—, —O—CO—,

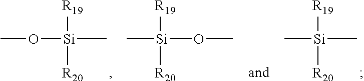

wherein R₁₂ and R₂₀ are independently selected from the group consisting of: -hydrogen, -aryl, -alkyl, -alkylene-aryl, alkoxy, and -alkylene-O-aryl;
R18 is -aryl, -alkyl, -alkylene-aryl, -alkylene-heteroaryl, or -alkylene-O-aryl;
Y3 and Y5 are independently selected from the group consisting of a direct bond, —CH2-, —N(H), —S—, SO₂—, —C(O)—, —CON(H)—, —NHC(O)—, —NHCON(H)—, —NHSO₂—, —SO₂N(H)—, —C(O)—O—, —NHSO₂NH—, —O—CO—,

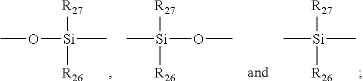

wherein R₂₇ and R₂₆ are independently selected from the group consisting of: -aryl, -alkyl, -alkylene-aryl, -alkoxy, and -alkyl-O-aryl;

### Y4 is

a) -alkylene,
b) -alkenylene,
c) -alkynylene,
d) -arylene,
e) -heteroarylene,
f) -cycloalkylene,
g) -heterocyclylene,
h) -alkylene-arylene,
i) -alkylene-heteroarylene,
j) -alkylene-cycloalkylene,
k) -alkylene-heterocyclylene,
l) -arylene-alkylene,
m) -heteroarylene-alkylene,
n) -cycloalkylene-alkylene,
o) -heterocyclylene-alkylene,

### p) —O—,

### q) —S—,

### r) —S(O)₂—, or

### s) —S(O)—,

wherein said alkylene groups may optionally contain one or more O, S, S(O), or SO₂ atoms;

### A2 is

a) heterocyclyl, fused arylheterocyclyl, or fused heteroarylheterocyclyl, containing at least one basic nitrogen atom, or
b) -imidazolyl, and
R₂₃, R₂₄, and R₂₅ are independently selected from the group consisting of: -hydrogen, -aryl, -heteroaryl, -alkylene-heteroaryl, -alkyl, -alkylene-aryl, -alkylene-O-aryl, and -alkylene-O-heteroaryl; and R₂₃ and R₂₄ may be taken together to form a five-membered ring having the formula —(CH₂)s-X3-(CH₂)t- bonded to the nitrogen atom to which R₂₃ and R₂₄ are attached
wherein

s and t are, independently, 1, 2, 3, or 4;

X3 is a direct bond, —CH₂—, —O—, —S—, —S(O)₂—, —C(O)—, —CON(H)—, —NHC(O)—, —NHCON(H)—, —NHSO₂—, —SO₂N(H)—, —C(O)—O—, —O—C(O)—, —NHSO₂NH—,

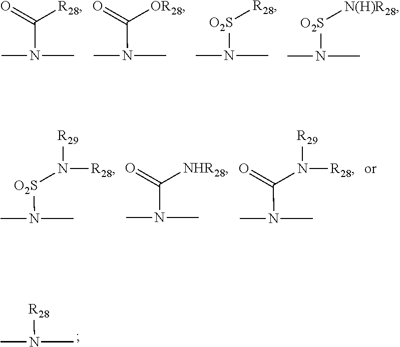

wherein R₂₈ and R₂₉ are independently selected from the group consisting of: -hydrogen, -aryl, -heteroaryl, -alkyl, -alkylene-aryl, and -alkylene-heteroaryl;
wherein the alkyl and/or aryl groups in the optional substituents
g) —Y2-alkyl,
h) —Y2-aryl,
i) —Y2-heteroaryl,
j) —Y2-alkylene-heteroaryl,
k) —Y2-alkylene-aryl,
l) —Y2-alkylene-W2-R₁₈,

### m) —Y3-Y4-NR₂₃R₂₄,

### n) —Y3-Y4-NH—C(═NR₂₅)NR₂₃R₂₄,

### o) —Y3-Y4-C(═NR₂₅)NR₂₃R₂₄, and

### p) —Y3-Y4-Y5-A2,

of R₂ and R₄ may be optionally substituted 1-4 times with a substituent independently selected from the group consisting of:
a) halogen,
b) perhaloalkyl,
c) alkyl,
d) cyano,
e) alkyloxy,
f) aryl, and
g) aryloxy, and
wherein the ring or rings containing a heteroatom in the heteroaryl, heteroarylene, heterocyclyl, heterocyclene, fused arylheterocyclyl, or fused heteroarylheterocyclyl groups in R₂ or R₄ or in a substituent of R₂ or R₄ is a five membered nitrogen containing ring, and
wherein
at least one of R₂ and R₄ is substituted with at least one group of the formula

—Y3-Y4-NR₂₃R₂₄,

—Y3-Y4-NH—C(═NR25)NR23R24,

—Y3-Y4-C(═NR25)NR23R24, or

—Y3-Y4-Y5-A2,

with the proviso that no more than one of R23, R24, and
R25 is aryl or heteroaryl;
or a pharmaceutically acceptable salt thereof.

In one embodiment, the antagonist is a compound having the structure:

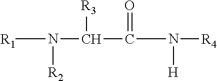

wherein
R1 and R2 are independently selected from

### a) —H;

b) —C1-6 alkyl;
c) -aryl;
d) —C1-6 alkylaryl;
e) —C(O)—O—C1-6 alkyl;
f) —C(O)—O—C1-6 alkylaryl;
h) —C(O)—NH—C1-6 alkylaryl;
i) —SO2-C1-6 alkyl;
j) —SO2-C1-6 alkylaryl;
k) —SO2-aryl;
l) —SO2-NH—C1-6 alkyl;
m) —SO2-NH—C1-6 alkylaryl;
n)

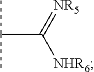

o) —C(O)—C1-6 alkyl; and
p) —C(O)—C1-6 alkylaryl;
R₃ is selected from
(a) -aryl; and
(b) —C1-3 alkylaryl,
wherein aryl is substituted by C1-6 alkyl, C1-6 alkoxy, C1-6 alkylaryl, or C1-6 alkoxyaryl;
   R4 is selected from

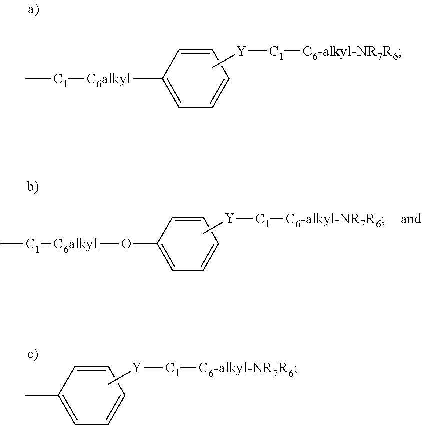

R5 and R6 are independently selected from the group consisting of hydrogen, C1-C6 alkyl, C1-C6 alkylaryl, and aryl; and wherein
the aryl and/or alkyl group(s) in R₁, R₂, R₄, R₅, R₆, R₇, R₈, R₉, R₁₀, R₁₈, R₁₉, and R₂₀ may be optionally substituted 1-4 times with a substituent group, wherein said substituent group(s) or the term substituted refers to groups selected from the group consisting of:

### a) —H;

b) —Y—C1-6 alkyl;

### —Y-aryl;

—Y—C1-6 alkylaryl;
—Y—C1-6-alkyl-NR7R8; and
—Y—C₁₋₆-alkyl-W—R20; and
c) halogen, hydroxyl, cyano, carbamoyl, or carboxyl; and
wherein
Y and W are independently selected from the group consisting of —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—CO—,

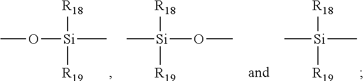

R₁₈ and R₁₉ are independently selected from the group consisting of aryl, C1-C6 alkyl, C1-C6 alkylaryl, C1-C6 alkoxy, and C1-C6 alkoxyaryl;
R₂₀ is selected from the group consisting of aryl, C1-C6 alkyl, and C1-C6 alkylaryl;
R₇, R₈, R₉ and R₁₀ are independently selected from the group consisting of hydrogen, aryl C1-C6 alkyl, and C1-C6 alkylaryl; and wherein
R₇ and R₈ may be taken together to form a ring having the formula —(CH₂)m-X—(CH₂)n- bonded to the nitrogen atom to which R₇ and R₈ are attached, and/or R₅ and R₆ may, independently, be taken together to form a ring having the formula —(CH₂)m-X—(CH₂)n- bonded to the nitrogen atoms to which R5 and R6 are attached, wherein m and n are, independently, 1, 2, 3, or 4; X is selected from the group consisting of —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—,

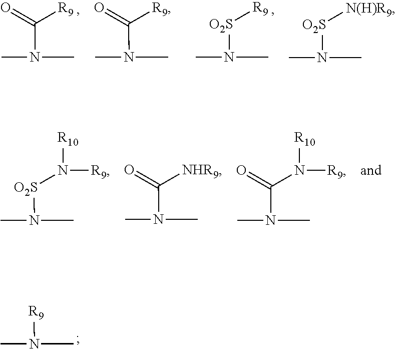

or a pharmaceutically acceptable salt thereof. It is understood that the above are all non-limiting examples of RAGE antagonists that can be employed in the present invention.

ROCK inhibitors (antagonists) include Fasudil, (5-(1,4-diazepane-1-sulfonyl)isoquinoline and the hydrochloride) Asahi-Kasei Pharmaceuticals, Inc., Japan, and Tocris Bioscience, Ellisville Mo.; Y27632, Mitsubishi Pharmaceuticals, Japan, and Y27632, Sigma Aldrich, USA; Y39983, Mitsubishi Pharmaceuticals, Japan; Senju Pharmaceuticals, Japan; and Novartis AG, Germany; Wf-536 Mitsubishi Pharmaceutical, Japan; SLx-2119, Surface Logix, Inc. USA; Azabenzimidazole-aminofurazans, Glaxo-Smith-Kline, UK; DE-104, olefins, isoquinolines, indazoles, pyridinealkene derivatives, Santen Pharmaceuticals, Japan; UBE Industries, Japan; H-1152P; Kowa Pharmaceuticals, Japan; ROKa inhibitor, BioFocus plc, USA; HMN-1152, Nagoya University, Japan; 4-(1-aminoalkyl)-N-(4-pyridyl)cyclohexanecarboxamides, BioAxone Therapeutics, Canada; Rhostatin, BioAxone Therapeutics, Canada; BA-210, BA-207, BA-215, BA-285, BA-1037, BioAxone Therapeutics, Canada; Ki-23095, Kirin Brewery Co., Japan; VAS-012, VasGene Therapeutics, USA; quinazoline ROCK inhibitor, Bayer AG, Germany. See Lioa, J K. Et al., J Cardiovasc Pharmacol (2007); 50:17-24, the contents of which are hereby incorporated by reference in their entirety.

As disclosed herein, ROCK indications may be treated using RAGE antagonists. The importance of ROCK in Kidney disease is described in Fu, P. J Am Soc Nephrol. 2006 November; 17(11):3105-14 which discusses a signaling mechanism of renal fibrosis in unilateral ureteral obstructive kidney disease in ROCK1 knockout mice. WO/2005/085469 discusses the role of ROCK in certain cardiovascular diseases, where cardiomyocyte apoptosis is present, and Chang J., Proc Natl Acad Sci USA. 2006 Sep. 26; 103(39):14495-500 discusses activation of Rho-. associated coiled-coil protein kinase 1 (ROCK-1) by caspase-3 cleavage playing an essential role in cardiac myocyte apoptosis. In Coudray A. M. et al., Int. J. Oncol. 2005 August; 27(2):553-61, increased anticancer activity of the chemotherapy agent thymidylate synthase inhibitor BGC9331 combined with the topoisomerase I inhibitor SN-38 in human colorectal and breast cancer cells was seen through induction of apoptosis and ROCK cleavage through caspase-3-dependent and -independent mechanisms. Rho-kinase (ROCK-1 and ROCK-2) were found upregulated in oleic acid-induced lung injury (Köksel O, Eur J Pharmacol. 2005 Mar. 7; 510(1-2):135-42), a model for Acute Respiratory Distress Syndrome. The role of ROCK in pancreatic inflammation and fibrosis is explained in Masamune A, Br J Pharmacol. 2003 December; 140(7):1292-302, Rho kinase inhibitors block activation of pancreatic stellate cells. In addition, Kaneko K., (Pancreas. 2002 April; 24(3):251-7) show expression of ROCK-1 in human pancreatic cancer, and its down-regulation by morpholino oligo antisense can reduce the migration of pancreatic cancer cells. All of the references cited in this paragraph are hereby incorporated by reference in their entirety.

As disclosed herein, RAGE indications may be treated using ROCK antagonists. Ishiguro H, (Prostate. 2005 Jun. 15; 64(1):92-100) describes that the receptor for advanced glycation end products (RAGE) and its ligand, amphoterin are overexpressed and associated with prostate cancer development. Hudson BI (Pharm Res. 2004 July; 21(7):1079-86) describe that RAGE is a novel target for drug intervention in diabetic vascular disease. Flyvbjerg A. describe the long-term renal effects of a neutralizing RAGE antibody in obese type 2 diabetic mice (Diabetes. 2004 January; 53(1):166-72). The role of RAGE in cancer is discussed in Riehl A, Cell Commun Signal. 2009 May 8; 7:12, “The receptor RAGE: Bridging inflammation and cancer” and Logsdon C D, Curr Mol Med. 2007 December; 7(8):777-89 “RAGE and RAGE ligands in cancer. The role of RAGE atherosclerosis and diabetes is discussed in Schmidt A M, Curr Atheroscler Rep. 2000 September; 2(5):430-6, “Atherosclerosis and diabetes: the RAGE connection”. Other RAGE indications are discussed in Koyama, H. et al. Arterioscler Thromb Vasc Biol. 2005 December; 25(12):2587-93 (Metabolic Syndrome); Katz, J. et al. J Periodontol. 2005 July (peridodontal disease, smoking-related); 76(7):1171-4; Cataldegirmen, G. et al. J Exp Med. 2005 Feb. 7; 201(3):473-84 (hepatocyte regeneration after partial hepatectomy); Sparvero, L. J. et al. J Transl Med. 2009 Mar. 17; 7:17; Raman, K. G. et al. Am J Physiol Gastrointest Liver Physiol. 2006 October; 291(4):G556-65 (intestinal barrier dysfunction); Yan, S. F. et al. Expert Rev Mol Med. 2009 Mar. 12; 11:e9 (neuropathy); and Yan, S.F. at al. J Mol. Med. 2009 March; 87(3):235-47 (nephropathy). All of the references cited in this paragraph are hereby incorporated by reference in their entirety.

“Treating” a disorder/disease shall mean slowing, stopping or reversing the disorder's progression, and/or ameliorating, lessening, or removing symptoms of the disorder. Thus treating a disorder encompasses reversing the disorder's progression, including up to the point of eliminating the disorder itself.

As used herein, an “immunoglobulin domain” is a sequence of amino acids that is structurally homologous, or identical to, a domain of an immunoglobulin. The length of the sequence of amino acids of an immunoglobulin domain may be up to 500 amino acids. In one embodiment, an immunoglobulin domain may be less than 250 amino acids. In an example embodiment, an immunoglobulin domain may be about 80-150 amino acids in length. For example, the variable region, and the CH1, CH2, and CH3 regions of an IgG (including human IgG) are each immunoglobulin domains. In another example, the variable, the CH1, CH2, CH3 and CH4 regions of an IgM are each immunoglobulin domains.

As used herein, a “RAGE immunoglobulin domain” is a sequence of amino acids from RAGE protein that is structurally homologous, or identical to, a domain of an immunoglobulin. For example, a RAGE immunoglobulin domain may comprise the RAGE V-domain, the RAGE Ig-like C2-type 1 domain (“C1 domain”), or the RAGE Ig-like C2-type 2 domain (“C2 domain”).

“Inflammatory vascular disease” shall mean a disease of the vascular or cardiovascular system of a mammal comprising an inflammatory response in a tissue of the vascular or cardiovascular system, for example a blood vessel thereof. In an embodiment, the disease is diabetic cardiovascular disease.

As used herein, “renal disease” means a pathological state affecting the normal physiological functioning of a mammalian kidney including, but not limited to, one or more of acute renal failure, chronic renal failure, abnormal renal transport syndromes, cystic kidney diseases, glomerular diseases, obstructive uropathies, and tubulointerstitial diseases, ureteral obstructive kidney disease and renal fibrosis.

As used herein, “lung disease” means a pathological state affecting the normal physiological functioning of a mammalian lung including chronic obstructive pulmonary disease, bronchitis, asthma and other inflammatory pulmonary diseases including those caused by environmental irritants, interstitial diseases, mediastinal and pleural disorders, bronchiectasis, and acute respiratory distress syndrome.

As used herein, “neurodegenerative disease” means a pathological state causing degradation in the normal physiological functioning of a mammalian neuron or collection of neurons (central or peripheral) including neuropathies, autoimmune neurodegenerative diseases such as multiple sclerosis, amyotrophic lateral sclerosis, Guillian-Barre syndrome, and central neurodegenerative such as Alzheimer's diseases.

As used herein, “cardiovascular disease” means a pathological state affecting the normal physiological functioning of a mammalian heart and/or the cardiac blood supply and/or other vascular components including arteriosclerosis, atherosclerosis, cardiomyopathies, coronary artery disease, peripheral vascular diseases, congestive heart failure, myocardial infarction, and ischemia/re-perfusion injury.

Further description of the various diseases recited in this disclosure may be found in The Merck Manual, 17th Edition (1999), Merck Research Laboratories, Whitehouse Station, N.J., U.S.A. which is hereby incorporated by reference for description of the diseases/disorders recited herein.

The administration of RAGE antagonsists or ROCK antagonists described herein may be by way of compositions containing one of the antagonists and a pharmacetically acceptable carrier. As used herein, a “pharmaceutical acceptable carrier” is a pharmaceutically acceptable solvent, suspending agent or vehicle, for delivering an active compound to a mammal, including humans. The carrier may be liquid, aerosol, gel or solid and is selected with the planned manner of administration in mind. In an embodiment, the pharmaceutical carrier is a sterile pharmaceutically acceptable solvent suitable for intravenous administration. In an embodiment, the pharmaceutical carrier is a pharmaceutically acceptable solid suitable for oral administration.

“Administering” the antagonists described herein can be effected or performed using any of the various methods and delivery systems known to those skilled in the art. The administering can be, for example, intravenous, oral, intramuscular, intravascular, intra-arterial, intracoronary, intramyocardial, intraperitoneal, and subcutaneous. Other non-limiting examples include via topical coating of a blood vessel, coating of a device to be placed within the subject, coating of an instrument used during a procedure which, for example, otherwise results in blood vessel injury, or contacting blood of the subject during extracorporeal circulation. In embodiments, administration is effected by injection or via a catheter.

Injectable drug delivery systems tha may be employed in the methods described herein include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention, i.e. a therapeutically effective amount. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Treatment of the diseases recited herein, e.g. of a cardiovascular disease, encompasses inducing inhibition, regression, or stasis of the disorder.

As used herein “about” with regard to a stated number encompasses a range of + one percent to − one percent of the stated value. By way of example, “100” therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101. Accordingly, “about 100” includes, in an embodiment, 100. Where a range is given in the specification it is understood that the range includes all integers and 0.1 units within that range, and any sub-range thereof. For example, a range of 77 to 90 includes 77, 78, 79, 80, and 81 etc., as well as 77 to 80 and 83-89, etc.

The methods of treatment described herein with the ROCK1 antagonist or RAGE antagonist may be a component of a combination therapy or an adjunct therapy. This combination therapy can be sequential therapy where the patient is treated first with one drug and then the other, or the two drugs are given simultaneously. These can be administered independently by the same route or by two or more different routes of administration depending on the dosage forms employed.

A method of treating a renal disease in a subject comprising administering to the subject an amount of an antagonist of receptor for advanced glycation end products (RAGE) effective to treat the renal disease in the subject.

In an embodiment the renal disease is ureteral obstructive kidney disease or renal fibrosis.

A method of treating a disease involving apoptosis of cardiomyocytes in a subject comprising administering to the subject an amount of an antagonist of receptor for advanced glycation end products (RAGE) effective to treat the disease involving apoptosis of cardiomyocytes in the subject.

A method of treating a lung disease in a subject comprising administering to the subject an amount of an antagonist of receptor for advanced glycation end products (RAGE) effective to treat the lung disease in the subject.

In an embodiment the lung disease is Acute Respiratory Distress Syndrome.

A method of enhancing the efficacy of a chemotherapeutic agent in inducing apoptosis of a tumor cell in a subject comprising administering to the subject a chemotherapeutic agent and an amount of an antagonist of receptor for advanced glycation end products (RAGE) effective to enhance the efficacy of the chemotherapeutic agent in inducing apoptosis of the tumor cell the subject.

In an embodiment the tumor cell is a colorectal cancer cell, a brain cancer cell, or a breast cancer cell.

In an embodiment the chemotherapeutic agent is thymidylate synthase inhibitor BGC9331 or topoisomerase I inhibitor SN-38.

A method of treating a colorectal cancer, breast cancer, or pancreatic cancer in a subject comprising administering to the subject an amount of an antagonist of receptor for advanced glycation end products (RAGE) effective to treat the colorectal cancer, breast cancer, or pancreatic cancer in the subject.

In an embodiment the cancer is pancreatic cancer.

A method of inhibiting metastasis of pancreatic cancer in a subject comprising administering to the subject an amount of an antagonist of receptor for advanced glycation end products (RAGE) effective to inhibit metastasis of the cancer in the subject.

In an embodiment the pancreatic cancer is a cancer of the pancreatic stellar cells.

A method of treating pancreatic inflammation in a subject comprising administering to the subject an amount of an antagonist of receptor for advanced glycation end products (RAGE) effective to treat pancreatic inflammation in the subject.

A method of treating cerebral vasospasm in a subject comprising administering to the subject an amount of an antagonist of receptor for advanced glycation end products (RAGE) effective to treat cerebral vasospasm in the subject.

A method of treating glaucoma in a subject comprising administering to the subject an amount of an antagonist of receptor for advanced glycation end products (RAGE) effective to treat glaucoma in the subject.

A method of treating tinnitus in a subject comprising administering to the subject an amount of an antagonist of receptor for advanced glycation end products (RAGE) effective to treat tinnitus in the subject.

A method of treating spinal cord injury in a subject comprising administering to the subject an amount of an antagonist of receptor for advanced glycation end products (RAGE) effective to treat spinal cord injury in the subject.

In an embodiment of the methods the antagonist is a RAGE antibody, a small molecule RAGE antagonist, a fusion protein RAGE antagonist, or a polypeptide RAGE antagonist.

In an embodiment of the methods the subject is a human subject.

A method of treating a neurodegenerative disease in a subject comprising administering to the subject an amount of an antagonist of rho-associated protein kinase 1 (ROCK1) effective to treat the neurodegenerative disease in the subject.

In an embodiment the neurodegenerative disease is Alzheimer's disease or multiple sclerosis.

A method of treating a diabetes-associated inflammatory disease in a subject comprising administering to the subject an amount of an antagonist of rho-associated protein kinase 1 (ROCK1) effective to treat the diabetes-associated inflammatory disease in the subject.

In an embodiment the diabetes-associated inflammatory disease is a diabetic neuropathy, a diabetic nephropathy, diabetic retinopathy, diabetic vascular disease, or diabetic atherosclerosis.

A method of treating a cardiovascular disease in a subject comprising administering to the subject an amount of an antagonist of rho-associated protein kinase 1 (ROCK1) effective to treat the cardiovascular disease in the subject.

In an embodiment the cardiovascular disease is congestive heart failure, myocardial infarction, ischemia/re-perfusion injury, or atherosclerosis.

A method of treating a vascular disease in a subject comprising administering to the subject an amount of an antagonist of rho-associated protein kinase 1 (ROCK1) effective to treat the vascular disease in the subject.

In an embodiment the vascular disease is a diabetic vascular disease, atherosclerosis, accelerated atherosclerosis, hyperlipidemic atherosclerosis, or peripheral vascular disease.

A method of treating a receptor for advanced glycation end products (RAGE)-associated inflammatory disease in a subject comprising administering to the subject an amount of an antagonist of rho-associated protein kinase 1 (ROCK1) effective to treat the RAGE-associated chronic inflammatory disease in the subject.

In an embodiment the RAGE-associated inflammatory disease is inflammatory bowel disease (IBD), is intestinal barrier dysfunction subsequent to hemorrhagic shock, or is seizure-induced neuronal damage.

A method of treating a renal cell carcinoma, prostate cancer, biliary cancer, or lung cancer in a subject comprising administering to the subject an amount of an antagonist of rho-associated protein kinase 1 (ROCK1) effective to treat the renal cell carcinoma, prostate cancer, biliary cancer, breast cancer or lung cancer in the subject.

In an embodiment the cancer is prostate cancer and is hormone refractory.

A method of promoting survival of a liver in a subject subsequent to a partial hepatectomy in the subject comprising administering to the subject before, after or during the partial hepatectomy an amount of an antagonist of rho-associated protein kinase 1 (ROCK1) effective to promote survival of the liver in the subject.

In an embodiment the amount of the ROCK1 antagonist is also effective to elicit hepatocyte regeneration in the liver of the subject.

A method of treating metabolic syndrome in a subject comprising administering to the subject an amount of an antagonist of rho-associated protein kinase 1 (ROCK1) effective to treat metabolic syndrome in the subject.

A method of treating obesity in a subject comprising administering to the subject an amount of an antagonist of rho-associated protein kinase 1 (ROCK1) effective to treat obesity in the subject.

A method of treating periodontal disease in a subject comprising administering to the subject an amount of an antagonist of rho-associated protein kinase 1 (ROCK1) effective to treat the periodontal disease in the subject.

In an embodiment the periodontal disease is smoking-related periodontal disease.

A method for treating hyperglycemia in a subject comprising administering to the subject an antagonist of an antagonist of rho-associated protein kinase 1 (ROCK1) in an amount effective to treat hyperglycemia in the subject.

A method for reducing levels of insulin in blood in a subject comprising administering to the subject an antagonist of rho-associated protein kinase 1 (ROCK1) in an amount effective to reduce insulin levels in blood in the subject.

A method for reducing levels of blood cholesterol in a subject comprising administering to the subject an antagonist of rho-associated protein kinase 1 (ROCK1) in an amount effective to reduce blood cholesterol levels in the subject.

A method for reducing levels of triglycerides in a subject comprising administering to the subject an antagonist of rho-associated protein kinase 1 (ROCK1) in an amount effective to reduce triglyceride levels in the subject.

A method for reducing levels of leptins in a subject comprising administering to the subject an antagonist of rho-associated protein kinase 1 (ROCK1) in an amount effective to reduce leptin levels in the subject.

A method for treating a subject with a condition associated with interaction of an amyloid-beta peptide with RAGE on a cell.

In an embodiment the condition is diabetes, Alzheimers' disease, senility, renal failure, hyperlipidemic atherosclerosis, neuronal cytotoxicity, dementia associated with head trauma, amyotrophic lateral sclerosis, multiple sclerosis or neuronal degeneration.

A method of treating a symptom of diabetes in a diabetic subject which comprises administering to the subject an antagonist of rho-associated protein kinase 1 (ROCK1) in an amount effective treat the symptom of diabetes in the subject.

In an embodiment the symptom is abnormal wound healing, a heart attack, a stroke, peripheral vascular disease, amputation, kidney disease, kidney failure, blindness, neuropathy, inflammation, exaggerated restenosis or impotence.

In an embodiment the symptom is abnormal wound healing and the method results in improved wound healing.

A method of alleviating a RAGE-associated inflammation in a subject which comprises administering to the subject an antagonist of rho-associated protein kinase 1 (ROCK1) in an amount effective treat the RAGE-associated inflammation in the subject.

In an embodiment the RAGE-associated inflammation is systemic lupus erythematosus, nephritis, vascular inflammation, arthritis, inflammatory colitis, chronic inflammatory bowel disease, asthma or ulcerative colitis.

A method of inhibiting metastasis of a non-pancreatic cancer in a subject comprising administering to the subject an antagonist of rho-associated protein kinase 1 (ROCK1) effective to inhibit metastasis of the non-pancreatic cancer in the subject.

A method of inhibiting new tissue growth in blood vessels in a subject, wherein the subject has experienced blood vessel injury, which comprises administering to the subject an antagonist of rho-associated protein kinase 1 (ROCK1) in an amount effective so as to inhibit new tissue growth in the subject's blood vessels.

A method of inhibiting neointimal formation in blood vessels in a subject, wherein the subject has experienced blood vessel injury, which comprises administering to the subject an antagonist of rho-associated protein kinase 1 (ROCK1) in an amount effective so as to inhibit neointimal formation in blood vessels in the subject's blood vessels.

A method of inhibiting the onset of glomerulosclerosis, proteinuria, or albunuria in a subject comprising administering to the subject a prophylactically effective amount of an antagonist of rho-associated protein kinase 1 (ROCK1) so as to thereby inhibit the onset of glomerulosclerosis, proteinuria, or albunuria in the subject.

A method for treating a RAGE-related disorder in a subject afflicted therewith comprising administering to the subject a therapeutically effective amount of an antagonist of rho-associated protein kinase 1 (ROCK1) so as to thereby treat the RAGE-related disorder.

In an embodiment the disorder is sepsis, atherosclerosis, multiple sclerosis, systemic lupus erythematosus, sepsis, transplant rejection, asthma, arthritis, tumor growth, cancer, metastasis of a cancer, complications due to diabetes, retinopathy, neuropathy, nephropathy, impotence, impaired wound healing, gastroparesis, Alzheimer's disease, Huntington's disease, amyotrophic lateral sclerosis, neointimal formation, amyloid angiopathy, inflammation, glomerular injury, seizure-induced neuronal damage, acute skin inflammation, chronic skin inflammation, psoriasis, atopic dermatitis, rheumatoid arthritis, lung inflammation, asthma, chronic obstructive pulmonary disease, diabetes, renal failure, hyperlipidemic atherosclerosis associated with diabetes, diabetic late complication increased vascular permeability, diabetic late complication nephropathy, diabetic late complication retinopathy, diabetic late complication neuropathy, neuronal cytotoxicity, amyotrophic lateral sclerosis, multiple sclerosis, dementia associated with head trauma, neuronal degeneration, restenosis, Down's syndrome, amyloidosis, periodontal disease or erectile dysfunction.

In an embodiment the RAGE-related disorder is retinopathy and the ROCK1 antagonist is administered to an eye of the subject.

In an embodiment the RAGE-related disorder is a nephropathy and the ROCK1 antagonist is administered via a catheter the subject

In an embodiment of the methods described herein the antagonist is a small molecule ROCK1 antagonist.

In an embodiment of the methods described herein the subject is a human subject.

A method for treating a ROCK1-related disorder in a subject afflicted therewith comprising administering to the subject a therapeutically effective amount of antagonist of receptor for advanced glycation end products (RAGE) so as to thereby treat the ROCK-related disorder.

In an embodiment the RAGE antagonist is a RAGE antibody, a small molecule RAGE antagonist, a fusion protein RACE antagonist, or a polypeptide RAGE antagonist.

In an embodiment of the methods described herein the subject is a human subject.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

To delineate the specific mechanisms by which RAGE accelerated early atherosclerosis, Affymetrix gene expression arrays were performed on aortas of non-diabetic and diabetic ApoE null mice expressing RAGE or devoid of RAGE at nine weeks of age, as this reflected a time point at which frank atherosclerotic lesions were not yet present, but at which genes likely involved in diabetes-dependent and RAGE-dependent atherogenesis would be identifiable. The data revealed that there is in fact very little overlap of the genes which are differentially expressed both in the onset of diabetes in ApoE null mice, and in the effect of RAGE deletion in diabetic ApoE null mice. Pathway-Express analysis revealed that the transforming growth factor-β pathway (tgf-β) and focal adhesion pathways would be expected to play a significant role in both the mechanism by which diabetes facilitates the formation of atherosclerotic plagues in ApoE null mice, and the mechanism by which deletion of RAGE ameliorates this effect. Quantitative polymerase chain reaction studies, Western blotting and confocal microscopy showed that RAGE-dependent acceleration of atherosclerosis in ApoE null mice is dependent, at least in part, on the action of the ROCK1 branch of the tgf-β family.

### Animal Studies

Male ApoE null mice in the C57BL/6 background were purchased from Jackson Laboratories. Homozygous RAGE null mice were backcrossed >12 generations into C57BL/6 prior to crossing with ApoE null mice to generate ApoE null/RAGE null breeding pairs. Mice were maintained at all times on a 12-hour light-dark cycle in a pathogen-free environment with free access to normal rodent chow and water.

All procedures were performed and approved by the Institutional Animal Care and Use Committee at Columbia University. Genomic DNA was isolated from tail biopsies, and PCR analysis was used to identify the deficiency of both genes ApoE and RAGE. At age 6 weeks, certain mice were rendered diabetic by administration of 5 daily intraperitoneal injections of streptozotocin, 65 mg/kg in citrate buffer (0.05 mol/L; pH 4.5) (Sigma Aldrich). Control mice were treated with citrate buffer alone. Serum glucose was measured from tail vein blood using a glucometer; serum glucose on at least two separate occasions of >250 mg/dl was considered diabetic state. Beginning at age 9 or 14 weeks, certain diabetic and nondiabetic mice were euthanized. Serum glucose was measured again before euthanasia to ensure that mice remained diabetic and the mice were weighed.

Four mice in each of the four categories were sampled at age 9 weeks for glucose, body weight, serum cholesterol, and RNA experiments, with the exception that 3 non-diabetic ApoE null/RAGE null mice were sampled. Western blotting was performed on all 4 mice in each group. An additional set of 4 mice per group was prepared for ROCK1 activation experiments. Ten mice each in the ApoE null and ApoE /RAGE null categories were initially made diabetic for experiments at 14 weeks, but, of these, only 8 ApoE null mice, and only 7 ApoE /RAGE null mice, remained diabetic at 14 weeks and were the subjects of glucose, weight, cholesterol, and atherosclerotic lesion experiments. Total aortic segments from root to bifurcation were snap-frozen for further analysis. Aortic roots were embedded into OCT (Tissue-Tek) and further sectioned for histological and immuohistochemical analysis.

Assessment of statistical significance of changes in serum glucose levels between different genotype and disease states at 9 and 14 weeks was determined using the 2 sample t-test. The statistical significance of the glucose concentration being above the diabetic threshold of 250 mg/ml, for nominally diabetic mice, and below the threshold for nominally non-diabetic mice, was determined using the 1 sample t-test. 95% confidence limits of the weight were calculated using the 1 sample t-test.

### Biochemical Analyses

Levels of total cholesterol were determined in plasma of mice fasted for at least 8 hrs prior to euthanasia using chromogenic assays (Thermo Electron Corporation). The statistical significance of changes in cholesterol between different genotype and disease at 9 and 14 weeks was determined using the 2 sample t-test (Ihaka R Gentleman R (1996) R: A language for data analysis and graphics. J Computational Graphical Statistics 5:299-314)

### Quantification of Atherosclerotic Lesion Area

The frozen sections from aortic roots were fixed in 10% buffered formalin for histology studies. Six 6-μm sections were collected at 80-μm intervals starting at a 100-μm distance from the appearance of the aortic valves. The sections were stained with Oil Red 0 and counterstained with hematoxylin. Atherosclerotic lesion areas were quantified using a Zeiss microscope and image analysis system (AxioVision 4.5). Four serial sections each was placed on 6 slides (total 24 sections), and mean lesion area was calculated by determining the mean lesion area of 1 section/slide for a total of 6 sections examined. The investigator was blinded to the experimental conditions. The statistical significance of changes in atherosclerotic lesion area between 14 week diabetic ApoE null and ApoE /RAGE null mice was determined using the 2 sample t-test.

### RNA Isolation and GeneChip Analysis.

High-quality RNA samples were extracted from the four groups of mice at age 9 weeks: diabetic ApoE null (n=4), non-diabetic ApoE null (n=4), diabetic ApoE null/RAGE null (n=4) and non-diabetic ApoE null/RAGE null aortas (n=3). RNA from 3 mice was employed in the last group secondary to failure to generate cRNA from one of the mice. Total aortic RNA was isolated from the indicated mice by using TRIzol (Invitrogen) and RNeasy MinElute Cleanup (QIAGEN Inc.) including a DNase step. Total RNA concentration and quality were assessed on a 2100 Bioanalyzer system (Agilent Technologies). All of the samples displayed an RNA integrity score >8, and there was no indication of RNA degradation or contamination with DNA. To prepare for expression analyses, cDNA was in vitro transcribed into biotin labeled antisense cRNA using an Affymetrix kit according to the standard kit protocol. 1 μg of RNA from each sample was hybridized to Affymetrix Mouse Genome 430 2.0 GeneChips (Gene Expression Omnibus Platform Accession number GPL1261). Arrays were scanned with GeneChip Scanner 3000-7G with GCOS software. Scanning was performed according to the protocol described in the Affymetrix GeneChip® Expression Analysis Technical Manual, November 2004 Edition.

All arrays in this study were normalized together using Robust Multiarray Average (RMA) (Irizarry R A, et al (2003) Summaries of Affymetrix GeneChip probe level data. Nucleic Acids Res 33 (Database Issue):D562-566). Log fold changes between conditions and the statistical significance of these fold changes were determined using contrasts in Linear Models for MicroArrays (LIMMA) (Smyth GK (2004) Linear Models and Empirical Bayes methods for assessing Differential Expression in Microarray Experiments. Statistical Applications in Genetics and Molecular Biology 3: Article 3, www.bepress.com/sagmb/vol3/iss1/art3/). Both RMA and LIMMA are implemented in Bioconductor (Gentleman RC, at al (2004) Bioconductor: open software development for computational biology and bioinformatics. Genome Biol 5: R80) which runs under R (Irizarry R A, et al (2003) Summaries of Affymetrix GeneChip probe level data. Nucleic Acids Res 33 (Database Issue):D562-566). The data have been deposited in the gene expression omnibus GEO (Barrett T, et al. (2005) NCBI GEO: mining millions of expression profiles-database and tools. Nucleic Acids Res 33(Database issue):D562-566), series accession number GSE15729. Accession numbers (Genelds) and gene symbols in NCBI Entrez Gene Database (where official symbols differ from that used in this paper, the official symbols are given first): Acta2/SMA(11475), Ager/RAGE (11596), ApoE(11816), Ltbp1(268977), PECAM1/CD31(18613), Ppp2r1b/PP2A(73699), Tgfbr1(21812), Tgfbr2(21813), RhoA (1848), Tgfb2/Tgf-b2 (21808), Thbs1(21825), Rock1(19877), and Smurf2(66313). Probesets were taken to be statistically significantly differentially expressed for a contrast between 2 conditions if their Bayesian log odds parameter, B>0. The Benjamini-Hochberg false discovery rate (P(BH)) (Benjamini Y & Hochberg Y (1995) Controlling the false discovery rate; A practical and powerful approach to multiple testing. J Roy. Stat. Soc. Ser B 57:289-300) is also given for comparison for select genes.

The KEGG Pathways (Kanehisa M, Goto S, Kawashima S, Okuno Y & Hattori M (2004) The KEGG resource for deciphering the genome. Nucleic Acids Res 32(Database issue):D277-280) associated with differential expression between conditions were identified with Pathway Express which identifies the pathways associated with differential expression in a way that takes pathway structure into account. Pathways with a gamma p-value calculated using the hypergeometric distribution and corrected for false discoveries of ≦0.05 are taken to be statistically significantly associated with the differential expression of a given contrast.

Real-Time RT-PCR validation

The differential expression of especially interesting genes was validated using RT-PCR. Total aortic RNA (0.5 μg) was reverse transcribed with SuperScript II according to the manufacturer's protocol (Invitrogen). After dilution of the cDNA to 50 μl, 1.5 μl of cDNA were amplified by real-time PCR on a sequence-detection system (Prism 7900HT1; ABI). ABI Assay-on-Demand kits containing primers and probes for mouse transforming growth factor-β2 (mTgfb2) (Mm01321738_m1), mouse thrombospondin-1 (mThbsl) (Mm01335418_m1), and mouse rho-associated protein kinase 1 (mROCK1) (Mm01225244_g1) were used. 18s rRNA was used as an endogenous reference to correct for differences in the amount of RNA.

Data were analyzed by the 2-ΔΔCT method. The statistical significance (p-values and 95% confidence limits) of these measurements were determined by the t-test as implemented in R (Ihaka R & Gentleman R (1996) R: A language for data analysis and graphics. J Computational Graphical Statistics 5:299-314). The t-test was applied to the count and log-fold change data so that reported 95% confidence limits for antilogs of counts and fold changes may be slightly asymmetric.

### Western Blot Analysis

Total lysate from mouse aorta was immunoblotted and probed with antibodies to Thbs1, TGF-β2 and ROCK1. Total lysate from mouse aorta was immunoblotted and probed with Thbs1-specific antibody (Lifespan biosciences, catalog #LS-C33686), TGF-β2-specific antibody (from Santa Cruz Biotechnology, catalog #sc-90) and ROCK1-specific antibody (Santa Cruz Biotechnology, catalog #sc-17794). HRP-conjugated donkey anti-rabbit IgG (Amersham Pharmacia Biotechnology, catalog #NA934) or HRP-conjugated sheep anti-mouse IgG (Amersham Pharmacia Biotechnology, catalog #NA931) was used to identify sites of binding of the primary antibody. After probing with the primary antibodies, membranes were stripped of bound immunoglobulins and reprobed with GAPDH (Abcam, catalog #ab8245). Blots were scanned with an Alfalmager TM 2200 scanner with AlfaEase (Alfalmager) FC 2200 software. Results are reported as a relative absorbance of test antigen to GAPDH.

### Immunohistochemistry

Acetone-fixed cryostat aortic sections were subjected to confocal microscopy for detection and merged images of RAGE, Thbs1, TGF-β2, and ROCK1 in endothelium and smooth muscle layers using specific antibodies to these two cell types and Bio-Rad Radiance 2000 Confocal System and the Lasersharp 2000 software (Bio-Rad). Acetone-fixed cryostat aortic sections were preincubated with CAS-BLOCK (Zymed; Invitrogen) for 30 minutes followed by avidin-biotin block for 15 minutes; sections were then subjected to incubation with primary rabbit polyclonal RAGE IgG, (Schmidt A M, et al. (1992) Isolation and characterization of two binding proteins for advanced glycosylation end products from bovine lung which are present on the endothelial cell surface. J. Biol. Chem. 267(21): 14987-14997; Hori O, et al. (1995) The receptor for advanced glycation end products (RAGE) is a cellular binding site for amphoterin. Mediation of neurite outgrowth and co-expression of RAGE and amphoterin in the developing nervous system. J Biol Chem 270(43): 25752-25761) and Thbs1, TGF-2, and ROCK1 antibodies as described above, followed by donkey anti-rabbit IgG as described above. Subsequently, Alexa Fluor 555 conjugate (Invitrogen) was incubated for 30 minutes. After washing, rat monoclonal CD31/PECAM1 antibody (Abcam, Catalog #ab7388) or mouse monoclonal smooth muscle actin (DakoCytomatin, CodeM0851) antibody were incubated for 1 hour followed by anti-rabbit or anti-mouse IgG, described above for 30 minutes, and then incubated with Alexa Fluor 488 conjugate (Invitrogen) for 30 minutes. Rabbit IgG (Zymed; Invitrogen) or omission of the primary antibody was used as a negative control. Slides were mounted with Vectorshield mounting media (Vector) and observed with an oil immersion objective using a Nikon E800 microscope. Images were collected using a Bio-Rad Radiance 2000 Confocal System and the Lasersharp 2000 software (Bio-Rad).

### ROCK1 Activity Assays

Activation of ROCK1 was evaluated on lysates of aorta or primary murine aortic smooth muscle cells retrieved from wild-type or RAGE null mice. In the latter case, SMCs were stimulated with S100B (10 μg/ml for the indicated times and subjected to ROCK1 activity assays (7-8). Aortas or SMCs were lysed using a Triton X-100 lysis buffer containing 50 mM Tris (pH 7.5), 10 mM MgCl2, 0.5 M NaCl and 1% Triton X-100. The samples were incubated with Anti-ROCK1 antibody described above for 1 hr further with Protein G coated agarose beads for overnight at 4° C., and the beads were washed three times with lysis buffer. The amount of ROCK1 associated with the beads was incubated with 50 μl kinase buffer, 1 μl 10 mM ATP (Cell Signalling) and 0.5 μg phosphorylated myosin phosphatase-1 (MYPT1)/protein phosphatase-1 regulatory (inhibitor) subunit 12A (Ppplrl2a) substrate (Upstate Biotech) at 300 C for 30 min. Reaction was stopped by adding sample buffer, boiled for 5 min and the amount of phosphorylated MYPT1 was examined by immunoblotting using ThrB50-phosphorylation specific antibody 36-003 (Upstate Biotechnology). After probing with the primary antibodies, membranes were stripped of bound immunoglobulins and reprobed with ROCK1 antibody as described above for normalization. Equal quantities of ROCK1 were treated with equal quantities of MYPT1/Ppp1r12a. Relative absorbances were measured as described above, and these were used to determine the relative activation of ROCK1. Statistical analysis was performed by the t-test and the results anti-log-transformed as above.

### Smooth Muscle Cell Migration and Proliferation Assays.

Wild-type and RAGE null mice vascular smooth muscle cells were isolated and cultured from the aorta and employed through passage 5 to 7. Migration assays were performed using the QCM Colorimetric Cell Migration Assay (Chemicon). Cells (3×105/well) were seeded into the upper chambers fitted with a lower 8 μm porous polycarbonate membrane, and the insert was placed in the lower chamber of a 24-well dish containing Dulbeccos' modified Eagle medium and no stimulant, S100B (10 μg/ml; generously provided by Dr. Guenter Fritz), Tgf-β2 (10 ng/ml; R&D Systems), or PDGF (10 ng/ml; R&D Systems) and incubated at 37° C. for 5 hrs and relative migration was measured according to the manufacturer's instructions. In experiments using anti-Tgf-β2 IgG and nonimmune IgG (10 μg/ml, both from Santa Cruz Biotechnology, Santa Cruz, Calif.), Y27632 (10 μM, Sigma Aldrich) and fasudil hydrochloride (10 μM, Tocris Bioscience, Ellisville Mo.) cells were preincubated with these agents or vehicle alone for 2 hrs in the case of antibodies and 30 mins in the case of Y27632 or fasudil at 37° C. prior to the addition to the chemotaxis chambers. Proliferation of cultured SMCs was quantified by measurement of incorporation of tritiated thymidine. SMCs were seeded at a density of 2×104 cells/wells in 24-well tissue culture-treated plates and incubated in serum-free DMEM for 16 hrs. Following a 2 hr preincubation with the indicated concentration of anti-Tgf-β2, nonimmune IgG, Y27632 or fasudil, cells were exposed to serum-free DMEM containing the indicated concentration of S100B or PDGF (10 ng/ml) along with 3H-thymidine (1 μCi/well). After the incubation period, cells were harvested 48 hrs later, and cellular proliferation was determined based on the incorporation of tritiated thymidine. Cell counting was performed and confirmed that increased trititated thymidine incorporation reflected an increase in cell number.

### Results

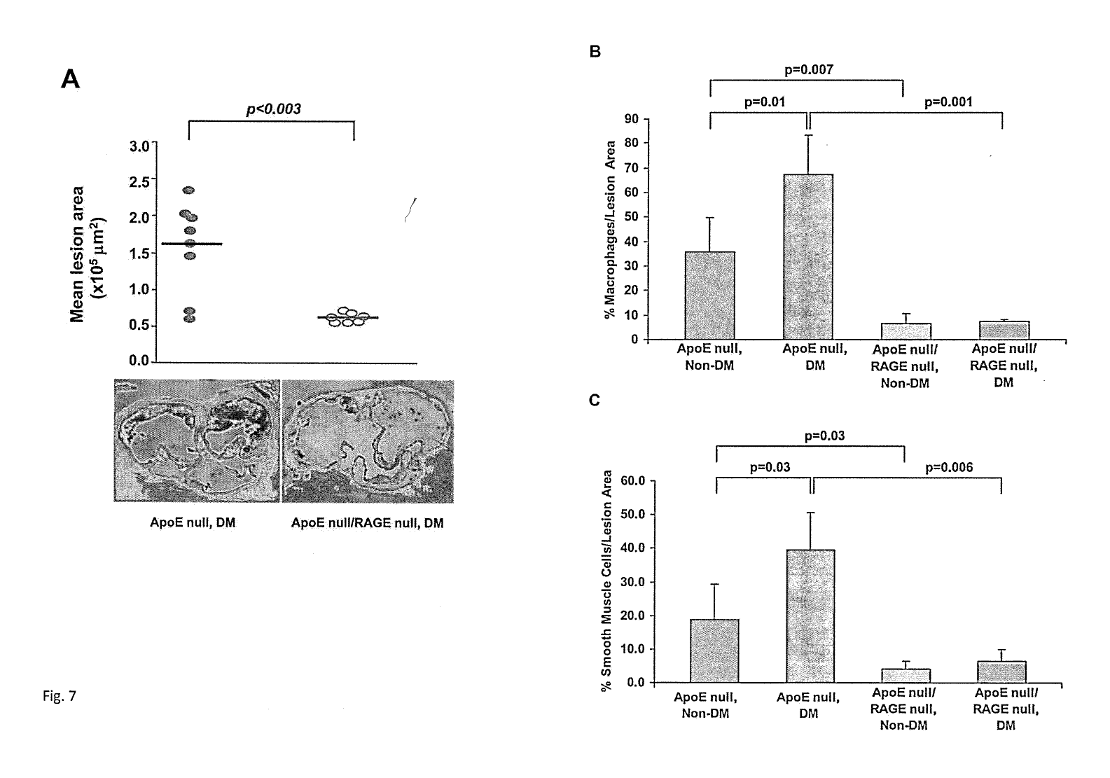

It was previously established that deletion of RAGE in non-diabetic ApoE null mice reduced atherosclerosis and vascular inflammation at age 14 weeks. To test these concepts in diabetes, studies were performed in RAGE-expressing or RAGE null ApoE null mice rendered diabetic at age 6 weeks. At 14 weeks of age, mean atherosclerotic lesion area at the aortic root in diabetic ApoE null mice was ≈2.8-fold higher in RAGE-expressing vs. RAGE-deficient ApoE null animals (1.59±0.23 vs. 0.57±0.03×10⁵ μm², respectively; p<0.003) (FIG. 7). Levels of serum glucose and cholesterol were not different between the two cohorts of diabetic mice (data not shown), suggesting that RAGE-dependent acceleration of diabetic atherosclerosis was not accounted for by glucose- or lipid-dependent factors.

Factors were examined that might account for the beneficial effects of RAGE deletion. Diabetic mice displayed a significantly higher plasma glucose level than non-diabetic mice, and importantly, there was no statistically significant dependence of the glucose concentration of either diabetic or non-diabetic mice on RAGE expression. The plasma cholesterol concentration and body weights of the mice in the various experimental conditions revealed no statistically significant dependence of cholesterol concentration or body weight on either genotype or disease state.

The lesion content of macrophages and smooth muscle cells (SMCs) was characterized by determining the percent (%) macrophages/lesion area and the % SMC/lesion area. At age 24 weeks, an age at which significant lesions would have formed in RAGE-expressing ApoE null mice, a significant increase in % macrophages/lesion area and % SMCs/lesion area was observed in diabetic ApoE null vs. non-diabetic ApoE null mice (FIGS. 7B & 7C, respectively). Consistent with important roles for RAGE in these processes, both diabetic and non-diabetic ApoE null/RAGE null mice displayed significantly decreased % macrophages/lesion area and % SMCs/lesion area compared to their respective RAGE-expressing ApoE null cohorts (FIGS. 7B & C, respectively).

These data suggested that RAGE contributed importantly to atherosclerosis in ApoE null mice in a manner independent of glucose, cholesterol or body weight, but in a manner linked to significant changes in lesion size and content. Thus, the specific mechanisms by which RAGE contributed to atherogenesis in ApoE null mice were sought. Accordingly, entire aortas were retrieved from non-diabetic and diabetic ApoE null mice at age 9 weeks, a time point at which the mice had not yet developed gross atherosclerotic plaques. RNA was prepared from individual aorta samples and subjected to Affymetrix gene arrays. Four comparisons of genome-wide differential expression between conditions were made. Each condition was defined by both its genotype and presence or absence of diabetes. The comparisons were as follows: 1. diabetic ApoE null relative to non-diabetic ApoE null; 2. non-diabetic ApoE null/RAGE null relative to non-diabetic ApoE null; 3. diabetic ApoE null/RAGE null relative to non-diabetic ApoE null/RAGE null; and 4. diabetic ApoE null/RAGE null relative to diabetic ApoE null aorta.

The number of unique genes with the Bayesian log-odds factor B>0 (indicating that the odds of differential expression is greater than 1) are reported. Only genes with Genbank symbols were counted, and genes with more than one probeset were only counted once. Using these parameters, it was reported that the onset of diabetes affects transcription in ApoE null mice (53 genes, comparison 1) much more than in ApoE null/RAGE null mice (3 genes, comparison 3), and that deletion of the RAGE gene in ApoE null mice affects transcription much more if the mice are diabetic (216 genes, comparison 4) than if they are non-diabetic (0 genes, comparison 2). Finally, more genes are affected by deletion of RAGE in diabetic ApoE null mice (216 genes, comparison 4) than by onset of diabetes in ApoE null mice (53 genes, comparison 1). Tables 1 and 2 show the log fold changes (log₂FC) and B values for all genes with B>0 for comparison 1 and comparison 4 respectively, the two comparisons with a non-negligible number of differentially expressed genes.

TABLE 1
Differentially Expressed Genes in Diabetic ApoE null Relative to Non-Diabetic ApoE null mice

| | | | log₂ | |
|---|---|---|---|---|
| Affymetrix ID | Gene Symbol | Gene Name | FC | B |
| 1423828_at | Fasn | fatty acid synthase | −2.33 | 4.36 |
| 1417877_at | 2310005P05Rik | RIKEN cDNA 2310005P05 gene | −0.63 | 2.96 |
| 1424542_at | S100a4 | S100 calcium binding protein A4 | 0.88 | 2.34 |
| 1416288_at | Dnaja1 | DnaJ (Hsp40) homolog, subfamily A, member 1 | 0.76 | 2.29 |
| 1425993_a_at | Hsp110 | heat shock protein 110 | 1.12 | 2.05 |
| 1419359_at | Hexim1 | hexamethylene bis-acetamide inducible 1 | 0.51 | 2.05 |
| 1434185_at | Acaca | acetyl-Coenzyme A carboxylase alpha | −1.57 | 1.81 |
| 1460179_at | Dnaja1 | DnaJ (Hsp40) homolog, subfamily A, member 1 | 0.69 | 1.71 |
| 1460645_at | Chordc1 | cysteine and histidine-rich domain (CHORD)-containing, zinc-binding protein 1 | 0.48 | 1.62 |
| 1416872_at | Tspan6 | tetraspanin 6 | 0.70 | 1.61 |
| 1427127_x_at | Hspa1b | heat shock protein 1B | 1.49 | 1.56 |
| 1460302_at | Thbs1 | thrombospondin 1 | 1.32 | 1.50 |
| 1423566_a_at | Hsp110 | heat shock protein 110 | 1.41 | 1.50 |
| 1437218_at | Fn1 | fibronectin 1 | 0.76 | 1.42 |
| 1460011_at | Cyp26b1 | cytochrome P450, family 26, subfamily b, polypeptide 1 | 0.70 | 1.40 |
| 1428190_at | Slc25a1 | solute carrier family 25 (mitochondrial carrier, citrate transporter), member 1 | −1.37 | 1.30 |
| 1456081_a_at | Aacs | acetoacetyl-CoA synthetase | −1.13 | 1.24 |
| 1428219_at | Rybp | RING1 and YY1 binding protein | 0.56 | 1.24 |
| 1452318_a_at | Hspa1b | heat shock protein 1B | 1.27 | 1.10 |
| 1439200_x_at | NA | NA | −1.75 | 1.00 |
| 1431802_a_at | D5Wsu178e | DNA segment, Chr 5, Wayne State University 178, expressed | 0.40 | 0.99 |
| 1428973_s_at | 0610012D17Rik | RIKEN cDNA 0610012D17 gene | 0.53 | 0.98 |
| 1422478_a_at | Acss2 | acyl-CoA synthetase short-chain family member 2 | −1.63 | 0.95 |
| 1423797_at | Aacs | acetoacetyl-CoA synthetase | −1.22 | 0.95 |
| 1427126_at | Hspa1b | heat shock protein 1B | 1.77 | 0.94 |
| 1448501_at | Tspan6 | tetraspanin 6 | 0.76 | 0.94 |
| 1451979_at | Kras | v-Ki-ras2 Kirsten rat sarcoma viral oncogene homolog | 0.43 | 0.93 |
| 1435337_at | Tshz3 | teashirt zinc finger family member 3 | 0.59 | 0.88 |
| 1424737_at | Thrsp | thyroid hormone responsive SPOT14 homolog (Rattus) | −1.16 | 0.88 |
| 1450541_at | Pvt1 | plasmacytoma variant translocation 1 | 0.54 | 0.88 |
| 1415860_at | Kpna2 | karyopherin (importin) alpha 2 | 0.53 | 0.79 |
| 1459874_s_at | Mtmr4 | myotubularin related protein 4 | 0.63 | 0.79 |
| 1448531_at | Lmnb2 | lamin B2 | 0.50 | 0.75 |
| 1416563_at | Ctps | cytidine 5′-triphosphate synthase | 0.73 | 0.74 |
| 1416529_at | Emp1 | epithelial membrane protein 1 | 0.64 | 0.68 |
| 1452805_at | D11Wsu47e | DNA segment, Chr 11, Wayne State University 47, expressed | 0.42 | 0.62 |
| 1449609_at | NA | NA | −0.40 | 0.61 |
| 1449528_at | Figf | c-fos induced growth factor | 0.61 | 0.59 |
| 1418659_at | Tparl | TPA regulated locus | 0.59 | 0.54 |
| 1452619_a_at | Agbl3 | ATP/GTP binding protein-like 3 | −0.43 | 0.51 |
| 1449044_at | Eef1e1 | eukaryotic translation elongation factor 1 epsilon 1 | 0.65 | 0.48 |
| 1418322_at | Crem | cAMP responsive element modulator | 0.61 | 0.48 |
| 1452406_x_at | Erdr1 | erythroid differentiation regulator 1 | −1.43 | 0.46 |
| 1439443_x_at | Tkt | transketolase | −0.79 | 0.43 |
| 1451015_at | Tkt | transketolase | −1.16 | 0.39 |
| 1447476_at | NA | NA | −0.37 | 0.39 |
| 1417196_s_at | Wwc2 | WW, C2 and coiled-coil domain containing 2 | 0.51 | 0.37 |
| 1435484_at | BF642829 | expressed sequence BF642829 | 0.80 | 0.37 |
| 1428989_at | 0710001D07Rik | RIKEN cDNA 0710001D07 gene | −0.84 | 0.36 |
| 1417741_at | Pygl | liver glycogen phosphorylase | −1.48 | 0.35 |
| 1435294_at | Mtmr11 | myotubularin related protein 11 | −0.37 | 0.30 |
| 1460330_at | Anxa3 | annexin A3 | 0.62 | 0.29 |
| 1428944_at | Ube1l2 | ubiquitin-activating enzyme E1-like 2 | 0.45 | 0.26 |
| 1416632_at | Mod1 | malic enzyme, supernatant | −1.20 | 0.25 |
| 1451666_at | Acly | ATP citrate lyase | −1.55 | 0.23 |
| 1454991_at | Slc7a1 | solute carrier family 7 (cationic amino acid transporter, y+ system), member 1 | 0.61 | 0.22 |
| 1456626_a_at | 1110005A23Rik | RIKEN cDNA 1110005A23 gene | 0.41 | 0.21 |
| 1422479_at | Acss2 | acyl-CoA synthetase short-chain family member 2 | −1.57 | 0.19 |
| 1452433_at | NA | NA | −0.46 | 0.18 |
| 1454993_a_at | Sfrs3 | splicing factor, arginine/serine-rich 3 (SRp20) | 0.46 | 0.16 |
| 1425326_at | Acly | ATP citrate lyase | −1.86 | 0.15 |
| 1418119_at | Rbm8a | RNA binding motif protein 8a | 0.41 | 0.14 |
| 1433897_at | AI597468 | expressed sequence AI597468 | 0.58 | 0.12 |
| 1460583_at | Golt1b | golgi transport 1 homolog B (S. cerevisiae) | 0.48 | 0.12 |
| 1416629_at | Slc1a5 | solute carrier family 1 (neutral amino acid transporter), member 5 | −0.59 | 0.11 |
| 1423796_at | Sfpq | splicing factor proline/glutamine rich (polypyrimidine tract binding protein associated) | 0.71 | 0.10 |
| 1435902_at | Nudt18 | nudix (nucleoside diphosphate linked moiety X)-type motif 18 | −0.41 | 0.09 |
| 1417460_at | Ifitm2 | interferon induced transmembrane protein 2 | 0.39 | 0.09 |
| 1450958_at | Tm4sf1 | transmembrane 4 superfamily member 1 | 0.64 | 0.09 |
| 1459835_s_at | Reln | reelin | 0.67 | 0.07 |
| 1436338_at | Ppp2r1b | protein phosphatase 2 (formerly 2A), regulatory subunit A (PR 65), beta isoform | −0.53 | 0.05 |
| 1440899_at | Fmo5 | flavin containing monooxygenase 5 | −0.48 | 0.05 |
| 1444560_at | NA | NA | 0.49 | 0.05 |
| 1425137_a_at | H2-Q10 | histocompatibility 2, Q region locus 10 | −1.34 | 0.04 |
| 1445894_at | NA | NA | 0.42 | 0.03 |
| 1427052_at | Acacb | acetyl-Coenzyme A carboxylase beta | −1.59 | 0.01 |

TABLE 2
Differentially Expressed Genes in Diabetic ApoE null/RAGE null Relative to Diabetic ApoE null mice

| Affymetrix ID | Gene Symbol | Gene Name | log₂ FC | B |
|---|---|---|---|---|
| 1452707_at | 4631423F02Rik | RIKEN cDNA 4631423F02 gene | −1.23 | 4.90 |
| 1439200_x_at | NA | NA | 2.45 | 4.43 |
| 1421834_at | Pip5k1a | phosphatidylinositol-4-phosphate 5-kinase, type 1 alpha | −0.82 | 4.28 |
| 1431413_at | Ramp1 | receptor (calcitonin) activity modifying protein 1 | −0.77 | 4.07 |
| 1435606_at | Gal3st4 | galactose-3-O-sulfotransferase 4 | −0.84 | 4.05 |
| 1460583_at | Golt1b | golgi transport 1 homolog B (S. cerevisiae) | −0.73 | 4.03 |
| 1447886_at | 0610040B09Rik | RIKEN cDNA 0610040B09 gene | −0.80 | 3.81 |
| 1419431_at | Ereg | epiregulin | −1.52 | 3.77 |
| 1450742_at | Bysl | bystin-like | −0.72 | 3.76 |
| 1426319_at | Pdgfd | platelet-derived growth factor, D polypeptide | −1.24 | 3.67 |
| 1416121_at | Lox | lysyl oxidase | −0.96 | 3.62 |
| 1452406_x_at | Erdr1 | erythroid differentiation regulator 1 | 1.96 | 3.56 |
| 1418322_at | Crem | cAMP responsive element modulator | −0.84 | 3.52 |
| 1456791_at | AA407452 | EST AA407452 | −0.50 | 3.35 |
| 1439849_at | NA | NA | −0.67 | 3.28 |
| 1425425_a_at | Wif1 | Wnt inhibitory factor 1 | −1.55 | 3.21 |
| 1447490_at | NA | NA | −1.49 | 3.20 |
| 1423250_a_at | Tgfb2 | transforming growth factor, beta 2 | −1.12 | 3.19 |
| 1418572_x_at | Tnfrsf12a | tumor necrosis factor receptor superfamily, member 12a | −1.41 | 3.13 |
| 1437498_at | NA | NA | −0.50 | 3.06 |
| 1436002_at | C230013L11Rik | RIKEN cDNA C230013L11 gene | −1.45 | 3.04 |
| 1429637_at | 2210419I08Rik | RIKEN cDNA 2210419I08 gene | −1.14 | 2.88 |
| 1458573_at | 9530026P05Rik | RIKEN cDNA 9530026P05 gene | 0.88 | 2.84 |
| 1417430_at | Cdr2 | cerebellar degeneration-related 2 | −0.99 | 2.77 |
| 1456532_at | Pdgfd | platelet-derived growth factor, D polypeptide | −0.81 | 2.71 |
| 1460645_at | Chordc1 | cysteine and histidine-rich domain (CHORD)-containing, zinc-binding protein 1 | −0.51 | 2.70 |
| 1424373_at | Armcx3 | armadillo repeat containing, X-linked 3 | −0.46 | 2.68 |
| 1448501_at | Tspan6 | tetraspanin 6 | −0.89 | 2.66 |
| 1427127_x_at | Hspa1b | heat shock protein 1B | −1.59 | 2.57 |
| 1439779_at | NA | NA | 0.56 | 2.55 |
| 1429564_at | Pcgf5 | polycomb group ring finger 5 | −0.69 | 2.52 |
| 1435337_at | Tshz3 | teashirt zinc finger family member 3 | −0.68 | 2.50 |
| 1440534_at | NA | NA | −1.20 | 2.50 |
| 1419149_at | Serpine1 | serine (or cysteine) peptidase inhibitor, clade E, member 1 | −1.88 | 2.47 |
| 1452318_a_at | Hspa1b | heat shock protein 1B | −1.41 | 2.42 |
| 1422307_at | NA | NA | −0.47 | 2.38 |
| 1452284_at | Ptprz1 | protein tyrosine phosphatase, receptor type Z, polypeptide 1 | −1.20 | 2.36 |
| 1422587_at | Tmem45a | transmembrane protein 45a | −0.77 | 2.36 |
| 1460011_at | Cyp26b1 | cytochrome P450, family 26, subfamily b, polypeptide 1 | −0.75 | 2.32 |
| 1424483_at | Mobk1b | MOB1, Mps One Binder kinase activator-like 1B (yeast) | −0.39 | 2.28 |
| 1433651_at | Wtip | WT1-interacting protein | −0.74 | 2.28 |
| 1446541_at | 4930434E21Rik | RIKEN cDNA 4930434E21 gene | −0.76 | 2.27 |
| 1448201_at | Sfrp2 | secreted frizzled-related protein 2 | −1.20 | 2.26 |
| 1433481_at | Fkbp14 | FK506 binding protein 14 | −0.79 | 2.23 |
| 1434572_at | Hdac9 | histone deacetylase 9 | −1.05 | 2.23 |
| 1433897_at | AI597468 | expressed sequence AI597468 | −0.72 | 2.12 |
| 1448892_at | Dock7 | dedicator of cytokinesis 7 | −0.73 | 2.12 |
| 1418571_at | Tnfrsf12a | tumor necrosis factor receptor superfamily, member 12a | −1.22 | 2.11 |
| 1435106_at | 3732412D22Rik | RIKEN cDNA 3732412D22 gene | −0.81 | 2.11 |
| 1430798_x_at | Mrpl15 | mitochondrial ribosomal protein L15 | 0.81 | 2.08 |
| 1448487_at | Lrrfip1 | leucine rich repeat (in FLII) interacting protein 1 | −0.80 | 2.07 |
| 1427484_at | Eml5 | echinoderm microtubule associated protein like 5 | 0.63 | 2.06 |
| 1420696_at | Sema3c | sema domain, immunoglobulin domain (Ig), short basic domain, secreted, (semaphorin) 3C | −0.89 | 2.05 |
| 1452257_at | Bdh1 | 3-hydroxybutyrate dehydrogenase, type 1 | −0.90 | 2.04 |
| 1425185_at | 5830417C01Rik | RIKEN cDNA 5830417C01 gene | −0.50 | 2.04 |
| 1435248_a_at | Btaf1 | BTAF1 RNA polymerase II, B-TFIID transcription factor-associated, | −0.48 | 2.04 |
| | | (Mot1 homolog, S. cerevisiae) | | |
| 1416288_at | Dnaja1 | DnaJ (Hsp40) homolog, subfamily A, member 1 | −0.70 | 2.03 |
| 1449065_at | Acot1 | acyl-CoA thioesterase 1 | 1.26 | 2.02 |
| 1428910_at | 2310022B05Rik | RIKEN cDNA 2310022B05 gene | −0.82 | 2.02 |
| 1450389_s_at | Pip5k1a | phosphatidylinositol-4-phosphate 5-kinase, type 1 alpha | −0.68 | 1.94 |
| 1435473_at | Gm347 | gene model 347, (NCBI) | −0.64 | 1.93 |
| 1440215_at | C130086A10 | NA | −0.79 | 1.93 |
| 1420688_a_at | Sgce | sarcoglycan, epsilon | −0.50 | 1.92 |
| 1438328_at | Hcfc2 | host cell factor C2 | −0.63 | 1.92 |
| 1448228_at | Lox | lysyl oxidase | −0.92 | 1.91 |
| 1448648_at | 9130005N14Rik | RIKEN cDNA 9130005N14 gene | −0.88 | 1.87 |
| 1448468_a_at | Kcnab1 | potassium voltage-gated channel, shaker-related subfamily, beta member 1 | −0.95 | 1.86 |
| 1427448_at | Rabep1 | rabaptin, RAB GTPase binding effector protein 1 | −0.39 | 1.86 |
| 1435574_at | NA | NA | −0.88 | 1.85 |
| 1418852_at | Chrna1 | cholinergic receptor, nicotinic, alpha polypeptide 1 (muscle) | −0.84 | 1.82 |
| 1427126_at | Hspa1b | heat shock protein 1B | −1.88 | 1.81 |
| 1417877_at | 2310005P05Rik | RIKEN cDNA 2310005P05 gene | 0.52 | 1.80 |
| 1439089_at | Zbtb41 | zinc finger and BTB domain containing 41 homolog | −0.49 | 1.79 |
| 1419595_a_at | Ggh | gamma-glutamyl hydrolase | −0.51 | 1.79 |
| 1421624_a_at | Enah | enabled homolog (Drosophila) | −1.00 | 1.77 |
| 1456483_at | Zfp9 | zinc finger protein 9 | −0.73 | 1.77 |
| 1451240_a_at | Glo1 | glyoxalase 1 | 0.81 | 1.76 |
| 1426306_a_at | Maged2 | melanoma antigen, family D, 2 | −0.84 | 1.76 |
| 1436319_at | Sulf1 | sulfatase 1 | −0.73 | 1.74 |
| 1444418_at | NA | NA | 0.60 | 1.72 |
| 1421833_at | Pip5k1a | phosphatidylinositol-4-phosphate 5-kinase, type 1 alpha | −0.50 | 1.72 |
| 1427019_at | Ptprz1 | protein tyrosine phosphatase, receptor type Z, polypeptide 1 | −0.99 | 1.70 |
| 1424607_a_at | Xdh | xanthine dehydrogenase | 0.59 | 1.69 |
| 1429417_at | 4833446K15Rik | RIKEN cDNA 4833446K15 gene | −1.18 | 1.67 |
| 1418349_at | Hbegf | heparin-binding EGF-like growth factor | −1.28 | 1.66 |
| 1434316_at | Chsy1 | carbohydrate (chondroitin) synthase 1 | −0.82 | 1.64 |
| 1460080_at | AI645535 | expressed sequence AI645535 | −0.55 | 1.63 |
| 1428638_at | Efhc2 | EF-hand domain (C-terminal) containing 2 | −1.07 | 1.62 |
| 1433529_at | E430002G05Rik | RIKEN cDNA E430002G05 gene | −0.92 | 1.61 |
| 1450416_at | Cbx5 | chromobox homolog 5 (Drosophila HP1a) | −0.51 | 1.60 |
| 1426782_at | Gpr125 | G protein-coupled receptor 125 | −0.65 | 1.59 |
| 1423347_at | Sec23a | SEC23A (S. cerevisiae) | −0.55 | 1.55 |
| 1452388_at | Hspa1a | heat shock protein 1A | −1.68 | 1.53 |
| 1434158_at | Gmds | GDP-mannose 4,6-dehydratase | −0.52 | 1.53 |
| 1425806_a_at | Surb7 | SRB7 (suppressor of RNA polymerase B) homolog (S. cerevisiae) | −0.43 | 1.52 |
| 1452238_at | Hrb | HIV-1 Rev binding protein | −0.55 | 1.52 |
| 1449584_at | Dgkg | diacylglycerol kinase, gamma | −0.75 | 1.49 |
| 1429888_a_at | Hspb2 | heat shock protein 2 | −0.92 | 1.49 |
| 1418538_at | Kdelr3 | KDEL (Lys-Asp-Glu-Leu) endoplasmic reticulum protein retention receptor 3 | −0.77 | 1.47 |
| 1436826_at | Tmtc3 | transmembrane and tetratricopeptide repeat containing 3 | −0.51 | 1.46 |
| 1452093_at | 2500001K11Rik | RIKEN cDNA 2500001K11 gene | −0.52 | 1.45 |
| 1418964_at | Pigm | phosphatidylinositol glycan, class M | −0.69 | 1.45 |
| 1427475_a_at | NA | NA | −0.70 | 1.44 |
| 1443939_at | LOC230628 | NA | −0.61 | 1.41 |
| 1450029_s_at | Itga9 | integrin alpha 9 | −1.00 | 1.41 |
| 1441989_at | Bnip2 | BCL2/adenovirus E1B interacting protein 1, NIP2 | −0.69 | 1.40 |
| 1417730_at | Ext1 | exostoses (multiple) 1 | −0.61 | 1.37 |
| 1458719_at | Glp1r | glucagon-like peptide 1 receptor | 1.91 | 1.36 |
| 1429232_at | 2610528B01Rik | RIKEN cDNA 2610528B01 gene | −0.48 | 1.35 |
| 1432417_a_at | Tspan2 | tetraspanin 2 | −1.09 | 1.33 |
| 1438004_at | Pols | polymerase (DNA directed) sigma | −0.82 | 1.30 |
| 1418802_at | R74862 | expressed sequence R74862 | −0.41 | 1.30 |
| 1422118_at | Sync | syncoilin | −0.78 | 1.29 |
| 1447547_at | Ltbp1 | latent transforming growth factor beta binding protein 1 | −1.31 | 1.28 |
| 1455316_x_at | Ccrn4l | CCR4 carbon catabolite repression 4-like (S. cerevisiae) | 0.57 | 1.28 |
| 1417272_at | 9130005N14Rik | RIKEN cDNA 9130005N14 gene | −0.60 | 1.28 |
| 1444396_at | Trp53inp2 | tumor protein p53 inducible nuclear protein 2 | 0.65 | 1.27 |
| 1456823_at | Gm70 | gene model 70, (NCBI) | −0.73 | 1.26 |
| 1422862_at | Pdlim5 | PDZ and LIM domain 5 | −0.85 | 1.24 |
| 1437523_s_at | Sgcg | sarcoglycan, gamma (dystrophin-associated glycoprotein) | −0.83 | 1.24 |
| 1457092_at | C630007B19Rik | RIKEN cDNA C630007B19 gene | −1.15 | 1.24 |
| 1422772_at | C1galt1 | core 1 UDP-galactose:N-acetylgalactosamine-alpha-R beta 1,3-galactosyltransferase | −0.88 | 1.20 |
| 1449063_at | Sec22l1 | SEC22 vesicle trafficking protein-like 1 (S. cerevisiae) | −0.40 | 1.20 |
| 1436101_at | Pank2 | pantothenate kinase 2 (Hallervorden-Spatz syndrome) | −0.74 | 1.20 |
| 1429065_at | 1200009F10Rik | RIKEN cDNA 1200009F10 gene | −0.79 | 1.19 |
| 1438566_at | St8sia6 | ST8 alpha-N-acetyl-neuraminide alpha-2,8-sialyltransferase 6 | 0.43 | 1.19 |
| 1419667_at | Sgcb | sarcoglycan, beta (dystrophin-associated glycoprotein) | −0.55 | 1.16 |
| 1427560_at | Six5 | sine oculis-related homeobox 5 homolog (Drosophila) | −0.45 | 1.16 |
| 1424609_a_at | Xdh | xanthine dehydrogenase | 0.57 | 1.15 |
| 1431693_a_at | Il17b | interleukin 17B | −0.73 | 1.14 |
| 1457817_at | Bcas3 | breast carcinoma amplified sequence 3 | 0.56 | 1.12 |
| 1435878_at | Stk38l | serine/threonine kinase 38 like | −0.82 | 1.12 |
| 1453172_at | Stch | stress 70 protein chaperone, microsome-associated, human homolog | −0.51 | 1.12 |
| 1417866_at | Tnfaip1 | tumor necrosis factor, alpha-induced protein 1 (endothelial) | −0.38 | 1.08 |
| 1455197_at | Rnd1 | Rho family GTPase 1 | −0.65 | 1.08 |
| 1423824_at | Gpr177 | G protein-coupled receptor 177 | −0.75 | 1.07 |
| 1454991_at | Slc7a1 | solute carrier family 7 (cationic amino acid transporter, y+ system), member 1 | −0.65 | 1.06 |
| 1439817_at | 2900064A13Rik | RIKEN cDNA 2900064A13 gene | −0.51 | 1.06 |
| 1417205_at | Kdelr2 | KDEL (Lys-Asp-Glu-Leu) endoplasmic reticulum protein retention receptor 2 | −0.63 | 1.06 |
| 1440435_at | Ky | kyphoscoliosis peptidase | −1.17 | 1.06 |
| 1450243_a_at | Dscr1l1 | Down syndrome critical region gene 1-like 1 | −1.60 | 1.02 |
| 1453087_at | 6330403L08Rik | RIKEN cDNA 6330403L08 gene | −0.72 | 1.01 |
| 1434883_at | Mtdh | Metadherin | −0.62 | 1.01 |
| 1426755_at | Ckap4 | cytoskeleton-associated protein 4 | −0.63 | 1.00 |
| 1450922_a_at | Tgfb2 | transforming growth factor, beta 2 | −1.25 | 0.99 |
| 1436203_a_at | 1110059G02Rik | RIKEN cDNA 1110059G02 gene | −0.92 | 0.97 |
| 1445421_at | NA | NA | −0.67 | 0.96 |
| 1421012_at | Srprb | signal recognition particle receptor, B subunit | −0.36 | 0.95 |
| 1428896_at | Pdgfrl | platelet-derived growth factor receptor-like | −0.75 | 0.92 |
| 1445848_at | LOC384500 | NA | 0.81 | 0.92 |
| 1448682_at | Dynll1 | dynein light chain LC8-type 1 | −0.64 | 0.92 |
| 1419015_at | Wisp2 | WNT1 inducible signaling pathway protein 2 | −0.82 | 0.92 |
| 1448556_at | Prlr | prolactin receptor | 0.67 | 0.91 |
| 1423405_at | Timp4 | tissue inhibitor of metalloproteinase 4 | 0.91 | 0.91 |
| 1424542_at | S100a4 | S100 calcium binding protein A4 | −0.71 | 0.90 |
| 1422437_at | Col5a2 | procollagen, type V, alpha 2 | −0.71 | 0.89 |
| 1431714_at | 2310015D24Rik | RIKEN cDNA 2310015D24 gene | −0.82 | 0.88 |
| 1452740_at | Myh10 | myosin, heavy polypeptide 10, non-muscle | −0.85 | 0.88 |
| 1435985_at | Stk25 | serine/threonine kinase 25 (yeast) | −0.54 | 0.85 |
| 1460302_at | Thbs1 | thrombospondin 1 | −1.17 | 0.84 |
| 1429508_at | 2310057M21Rik | RIKEN cDNA 2310057M21 gene | −0.64 | 0.84 |
| 1458566_at | Gpatc2 | G patch domain containing 2 | 0.54 | 0.83 |
| 1449096_at | 0610011N22Rik | RIKEN cDNA 0610011N22 gene | −0.55 | 0.83 |
| 1437902_s_at | Lrrc61 | leucine rich repeat containing 61 | −0.51 | 0.82 |
| 1416174_at | Rbbp9 | retinoblastoma binding protein 9 | −0.43 | 0.82 |
| 1433934_at | Sec24a | SEC24 related gene family, member A (S. cerevisiae) | −0.42 | 0.82 |
| 1418820_s_at | Zcchc10 | zinc finger, CCHC domain containing 10 | −0.49 | 0.81 |
| 1416441_at | Pgcp | plasma glutamate carboxypeptidase | −0.60 | 0.80 |
| 1451975_at | 2810453I06Rik | RIKEN cDNA 2810453I06 gene | −0.54 | 0.79 |
| 1415988_at | Hdlbp | high density lipoprotein (HDL) binding protein | −0.55 | 0.79 |
| 1448251_at | 9030425E11Rik | RIKEN cDNA 9030425E11 gene | −0.88 | 0.79 |
| 1423316_at | Tmem39a | transmembrane protein 39a | −0.74 | 0.78 |
| 1437370_at | Sgol2 | shugoshin-like 2 (S. pombe) | 0.45 | 0.76 |
| 1459495_at | NA | NA | −0.50 | 0.75 |
| 1448207_at | Lasp1 | LIM and SH3 protein 1 | −0.45 | 0.75 |
| 1420636_a_at | Dusp12 | dual specificity phosphatase 12 | −0.42 | 0.73 |
| 1460260_s_at | Kpna1 | karyopherin (importin) alpha 1 | −0.39 | 0.72 |
| 1415741_at | Tparl | TPA regulated locus | −0.52 | 0.71 |
| 1442148_at | Psip1 | PC4 and SFRS1 interacting protein 1 | −0.58 | 0.71 |
| 1454842_a_at | B3galnt2 | UDP-GalNAc:betaGlcNAc beta 1,3-galactosaminyltransferase, polypeptide 2 | −0.69 | 0.69 |
| 1416554_at | Pdlim1 | PDZ and LIM domain 1 (elfin) | −0.84 | 0.67 |
| 1442257_at | NA | NA | −1.03 | 0.67 |
| 1455583_at | Gne | glucosamine | −0.44 | 0.67 |
| 1426468_at | 0610037L13Rik | RIKEN cDNA 0610037L13 gene | −0.36 | 0.66 |
| 1450784_at | Reck | reversion-inducing-cysteine-rich protein with kazal motifs | −0.49 | 0.66 |
| 1440397_at | Cacna2d1 | calcium channel, voltage-dependent, alpha2/delta subunit 1 | −0.70 | 0.66 |
| 1434950_a_at | Armc8 | armadillo repeat containing 8 | −0.41 | 0.65 |
| 1435641_at | 9530018I07Rik | RIKEN cDNA 9530018I07 gene | −0.73 | 0.64 |
| 1418455_at | Copz2 | coatomer protein complex, subunit zeta 2 | −0.47 | 0.63 |
| 1451629_at | Lbh | limb-bud and heart | −0.70 | 0.63 |
| 1450994_at | Rock1 | Rho-associated coiled-coil forming kinase 1 | −0.66 | 0.60 |
| 1456798_at | 9330118A15Rik | RIKEN cDNA 9330118A15 gene | −0.78 | 0.60 |
| 1430479_at | 2010007H06Rik | RIKEN cDNA 2010007H06 gene | 0.43 | 0.60 |
| 1428983_at | Scx | scleraxis | −0.77 | 0.60 |
| 1432494_a_at | 1700019E19Rik | RIKEN cDNA 1700019E19 gene | −0.73 | 0.60 |
| 1425921_a_at | 1810055G02Rik | RIKEN cDNA 1810055G02 gene | −0.66 | 0.58 |
| 1431079_at | C1qtnf2 | C1q and tumor necrosis factor related protein 2 | −0.81 | 0.58 |
| 1425913_a_at | 2810022L02Rik | RIKEN cDNA 2810022L02 gene | −0.80 | 0.58 |
| 1437259_at | Slc9a2 | solute carrier family 9 (sodium/hydrogen exchanger), member 2 | −0.67 | 0.57 |
| 1433670_at | Emp2 | epithelial membrane protein 2 | −0.55 | 0.57 |
| 1424568_at | Tspan2 | tetraspanin 2 | −0.78 | 0.56 |
| 1450728_at | Fjx1 | four jointed box 1 (Drosophila) | −0.73 | 0.56 |
| 1423352_at | Crispld1 | cysteine-rich secretory protein LCCL domain containing 1 | −0.52 | 0.56 |
| 1433543_at | Anln | anillin, actin binding protein (scraps homolog, Drosophila) | −0.95 | 0.55 |
| 1445969_at | NA | NA | −0.65 | 0.54 |
| 1418722_at | Ngp | neutrophilic granule protein | 0.53 | 0.53 |
| 1441904_x_at | 9130005N14Rik | RIKEN cDNA 9130005N14 gene | −0.63 | 0.53 |
| 1416686_at | Plod2 | procollagen lysine, 2-oxoglutarate 5-dioxygenase 2 | −0.78 | 0.53 |
| 1450943_at | 2010012C16Rik | RIKEN cDNA 2010012C16 gene | −0.56 | 0.53 |
| 1428219_at | Rybp | RING1 and YY1 binding protein | −0.50 | 0.53 |
| 1453993_a_at | Bnip2 | BCL2/adenovirus E1B interacting protein 1, NIP2 | −0.55 | 0.53 |
| 1428538_s_at | Rarres2 | retinoic acid receptor responder (tazarotene induced) 2 | −0.57 | 0.51 |
| 1433929_at | Nhlrc2 | NHL repeat containing 2 | −0.40 | 0.51 |
| 1418413_at | Cav3 | caveolin 3 | −0.75 | 0.50 |
| 1460203_at | Itpr1 | inositol 1,4,5-triphosphate receptor 1 | −0.57 | 0.49 |
| 1424556_at | Pycr1 | pyrroline-5-carboxylate reductase 1 | −0.98 | 0.49 |
| 1426540_at | Endod1 | endonuclease domain containing 1 | −0.83 | 0.48 |
| 1419169_at | Mapk6 | mitogen-activated protein kinase 6 | −0.46 | 0.48 |
| 1455294_at | 1110029L17Rik | RIKEN cDNA 1110029L17 gene | −0.36 | 0.48 |
| 1417104_at | Emp3 | epithelial membrane protein 3 | −0.52 | 0.47 |
| 1452521_a_at | Plaur | plasminogen activator, urokinase receptor | −0.59 | 0.46 |
| 1455320_at | Pbef1 | pre-B-cell colony-enhancing factor 1 | 0.76 | 0.45 |
| 1416165_at | Rab31 | RAB31, member RAS oncogene family | −0.51 | 0.45 |
| 1422818_at | Nedd9 | neural precursor cell expressed, developmentally down-regulated gene 9 | −0.90 | 0.44 |
| 1437637_at | Phtf2 | putative homeodomain transcription factor 2 | −0.98 | 0.44 |
| 1418454_at | Mfap5 | microfibrillar associated protein 5 | −0.60 | 0.44 |
| 1435981_at | NA | NA | −0.70 | 0.43 |
| 1424800_at | Enah | enabled homolog (Drosophila) | −0.77 | 0.43 |
| 1437101_at | Lats2 | large tumor suppressor 2 | −0.65 | 0.43 |
| 1429823_at | 5430420E18Rik | RIKEN cDNA 5430420E18 gene | −0.95 | 0.42 |
| 1428668_at | Acbd3 | acyl-Coenzyme A binding domain containing 3 | −0.45 | 0.42 |
| 1427730_a_at | Zfp148 | zinc finger protein 148 | −0.34 | 0.41 |
| 1415758_at | Fryl | furry homolog-like (Drosophila) | −0.40 | 0.41 |
| 1443916_at | 2900026A02Rik | RIKEN cDNA 2900026A02 gene | −0.55 | 0.40 |
| 1443501_at | NA | NA | 0.44 | 0.40 |
| 1452094_at | P4ha1 | procollagen-proline, 2-oxoglutarate 4-dioxygenase (proline 4-hydroxylase), alpha 1 | −0.77 | 0.40 |
| | | polypeptide | | |
| 1423440_at | 1110001A07Rik | RIKEN cDNA 1110001A07 gene | −0.37 | 0.39 |
| 1423566_a_at | Hsp110 | heat shock protein 110 | −1.19 | 0.39 |
| 1417570_at | Anapc1 | anaphase promoting complex subunit 1 | −0.83 | 0.39 |
| 1431166_at | Chd1 | chromodomain helicase DNA binding protein 1 | 0.57 | 0.38 |
| 1455375_at | NA | NA | −0.84 | 0.38 |
| 1425993_a_at | Hsp110 | heat shock protein 110 | −0.88 | 0.37 |
| 1454876_at | Rab23 | RAB23, member RAS oncogene family | −0.67 | 0.37 |
| 1424801_at | Enah | enabled homolog (Drosophila) | −0.78 | 0.37 |
| 1448810_at | Gne | glucosamine | −0.37 | 0.37 |
| 1423649_at | Tmem68 | transmembrane protein 68 | −0.35 | 0.37 |
| 1458667_at | 4930519N13Rik | RIKEN cDNA 4930519N13 gene | 0.48 | 0.36 |
| 1450923_at | Tgfb2 | transforming growth factor, beta 2 | −1.09 | 0.36 |
| 1436737_a_at | Sorbs1 | sorbin and SH3 domain containing 1 | 0.46 | 0.35 |
| 1431340_a_at | 2310002J21Rik | RIKEN cDNA 2310002J21 gene | −0.46 | 0.35 |
| 1422919_at | Hrasls | HRAS-like suppressor | −0.59 | 0.35 |
| 1436178_at | Leprel1 | leprecan-like 1 | −0.85 | 0.35 |
| 1431131_s_at | A630007B06Rik | RIKEN cDNA A630007B06 gene | −0.35 | 0.35 |
| 1415856_at | Emb | embigin | −0.88 | 0.33 |
| 1444289_at | Yipf5 | Yip1 domain family, member 5 | −0.73 | 0.32 |
| 1420855_at | Eln | elastin | −0.89 | 0.32 |
| 1424318_at | 1110067D22Rik | RIKEN cDNA 1110067D22 gene | −0.47 | 0.31 |
| 1417629_at | Prodh | proline dehydrogenase | 1.13 | 0.30 |
| 1423790_at | Dap | death-associated protein | −0.52 | 0.30 |
| 1454916_s_at | Arfip1 | ADP-ribosylation factor interacting protein 1 | −0.41 | 0.29 |
| 1423841_at | Bxdc2 | brix domain containing 2 | −0.67 | 0.29 |
| 1451453_at | Dapk2 | death-associated kinase 2 | −0.62 | 0.29 |
| 1434802_s_at | Ntf3 | neurotrophin 3 | −0.69 | 0.28 |
| 1457042_at | AI256396 | EST AI256396 | −0.85 | 0.28 |
| 1434787_at | Arf3 | ADP-ribosylation factor 3 | −0.43 | 0.27 |
| 1451469_at | D530005L17Rik | RIKEN cDNA D530005L17 gene | −0.49 | 0.27 |
| 1418792_at | Sh3gl2 | SH3-domain GRB2-like 2 | 0.39 | 0.26 |
| 1453189_at | Ube2i | ubiquitin-conjugating enzyme E2I | 0.47 | 0.26 |
| 1418350_at | Hbegf | heparin-binding EGF-like growth factor | −1.32 | 0.26 |
| 1452283_at | Rassf8 | Ras association (RalGDS/AF-6) domain family 8 | −0.71 | 0.25 |
| 1436181_at | Itgb1bp1 | integrin beta 1 binding protein 1 | −0.77 | 0.25 |
| 1438271_at | Lpp | LIM domain containing preferred translocation partner in lipoma | −0.64 | 0.25 |
| 1422568_at | Ndel1 | nuclear distribution gene E-like homolog 1 (A. nidulans) | −0.35 | 0.24 |
| 1452968_at | Cthrc1 | collagen triple helix repeat containing 1 | −0.53 | 0.23 |
| 1452145_at | H6pd | hexose-6-phosphate dehydrogenase (glucose 1-dehydrogenase) | 0.46 | 0.23 |
| 1433761_at | NA | NA | −0.63 | 0.22 |
| 1450079_at | Nrk | Nik related kinase | −0.85 | 0.22 |
| 1456415_at | Zfp451 | zinc finger protein 451 | −0.79 | 0.21 |
| 1430259_at | Tnfrsf11a | tumor necrosis factor receptor superfamily, member 11a | −0.47 | 0.21 |
| 1428644_at | Mgat5 | mannoside acetylglucosaminyltransferase 5 | −0.65 | 0.20 |
| 1455570_x_at | Cnn3 | calponin 3, acidic | −0.60 | 0.20 |
| 1423825_at | Gpr177 | G protein-coupled receptor 177 | −0.57 | 0.20 |
| 1456626_a_at | 1110005A23Rik | RIKEN cDNA 1110005A23 gene | −0.40 | 0.20 |
| 1435160_at | 1110064P04Rik | RIKEN cDNA 1110064P04 gene | −0.49 | 0.19 |
| 1434869_at | Tdrd3 | tudor domain containing 3 | −0.48 | 0.19 |
| 1455862_at | 9630054F20Rik | RIKEN cDNA 9630054F20 gene | −0.63 | 0.18 |
| 1437268_at | Lancl3 | LanC lantibiotic synthetase component C-like 3 (bacterial) | −0.88 | 0.18 |
| 1423033_at | Stt3a | STT3, subunit of the oligosaccharyltransferase complex, homolog A (S. cerevisiae) | −0.57 | 0.17 |
| 1426584_a_at | Sord | sorbitol dehydrogenase | 0.40 | 0.16 |
| 1439837_at | Tnrc15 | trinucleotide repeat containing 15 | −0.58 | 0.15 |
| 1429045_at | Smurf2 | SMAD specific E3 ubiquitin protein ligase 2 | −0.43 | 0.15 |
| 1456763_at | AA536749 | expressed sequence AA536749 | −0.46 | 0.15 |
| 1436297_a_at | Grina | glutamate receptor, ionotropic, N-methyl D-asparate-associated protein 1 (glutamate binding) | 0.60 | 0.13 |
| 1452770_at | Vkorc1 | vitamin K epoxide reductase complex, subunit 1 | −0.44 | 0.13 |
| 1423915_at | Olfml2b | olfactomedin-like 2B | −0.66 | 0.13 |
| 1421818_at | Bcl6 | B-cell leukemia/lymphoma 6 | −0.83 | 0.12 |
| 1429027_at | 0610007N19Rik | RIKEN cDNA 0610007N19 gene | −0.60 | 0.12 |
| 1434958_at | Sacs | sacsin | −0.83 | 0.12 |
| 1428097_at | 2510009E07Rik | RIKEN cDNA 2510009E07 gene | −0.55 | 0.11 |
| 1434078_at | D7Wsu128e | DNA segment, Chr 7, Wayne State University 128, expressed | −0.40 | 0.11 |
| 1423136_at | Fgf1 | fibroblast growth factor 1 | −0.49 | 0.10 |
| 1422644_at | Sh3bgr | SH3-binding domain glutamic acid-rich protein | −0.78 | 0.10 |
| 1443550_at | NA | NA | −0.33 | 0.08 |
| 1417965_at | Plekha1 | pleckstrin homology domain containing, family A (phosphoinositide binding specific) member 1 | −0.63 | 0.07 |
| 1449324_at | Ero1l | ERO1-like (S. cerevisiae) | −0.48 | 0.07 |
| 1431381_at | 3110005L24Rik | RIKEN cDNA 3110005L24 gene | 0.71 | 0.07 |
| 1437396_at | Creb3l2 | cAMP responsive element binding protein 3-like 2 | −0.61 | 0.06 |
| 1421260_a_at | Srm | spermidine synthase | −0.35 | 0.05 |
| 1436204_at | 1110059G02Rik | RIKEN cDNA 1110059G02 gene | −0.73 | 0.04 |
| 1454043_a_at | Kcnab1 | potassium voltage-gated channel, shaker-related subfamily, beta member 1 | −1.44 | 0.04 |
| 1455779_at | Hisppd2a | histidine acid phosphatase domain containing 2A | −0.62 | 0.04 |
| 1444246_at | Chd2 | chromodomain helicase DNA binding protein 2 | 0.44 | 0.03 |
| 1425066_a_at | 1110061O04Rik | RIKEN cDNA 1110061O04 gene | 0.64 | 0.03 |
| 1440484_at | Unc5d | unc-5 homolog D (C. elegans) | −0.50 | 0.03 |
| 1416749_at | Htra1 | HtrA serine peptidase 1 | −0.63 | 0.03 |
| 1416469_at | Luzp1 | leucine zipper protein 1 | −0.40 | 0.02 |
| 1433877_at | 4732473B16Rik | RIKEN cDNA 4732473B16 gene | −0.78 | 0.01 |
| 1438993_a_at | Atp6v1d | ATPase, H+ transporting, lysosomal V1 subunit D | 0.42 | 0.01 |

Pathway-Express analysis was performed on the gene lists in Tables 1 and 2 to determine the pathways that were most associated with the onset of diabetes in ApoE null mice and the effect of RAGE gene deletion in diabetic ApoE null mice. Statistically significant pathways (with a gamma p-value corrected for false discoveries ≦0.05) are listed in Tables 3 and 4. Tgf-β2 and focal adhesion pathways are common to both lists, suggesting that these pathways play a significant role in both the mechanism by which diabetes facilitates the formation of atherosclerotic plaques in ApoE null mice, and the mechanism by which deletion of RAGE ameliorates this effect. Thus, the Tgf-β pathway was focused on because of the established role for this pathway in atherogenesis (9-15).

TABLE 3
Statistically Significant KEGG Pathways Associated with
Differentially-Expressed Genes for Diabetic ApoE
null Relative to Non-Diabetic ApoE null mice

| | | Number of | Total | False |
|---|---|---|---|---|
| | | differentially | number of | discovery |
| | | expressed | genes in | rate |
| | | genes in | KEGG | corrected |
| | | KEGG | pathway | gamma |
| Rank | Pathway Name | pathway | on chip | p-value |
| 1 | Insulin signaling pathway | 5 | 137 | 1.5E−03 |
| 2 | Bladder cancer | 3 | 42 | 9.6E−03 |
| 3 | Cell Communication | 4 | 130 | 0.01 |
| 4 | ECM-receptor interaction | 3 | 84 | 0.01 |
| 5 | Focal adhesion | 4 | 192 | 0.01 |
| 6 | TGF-beta signaling | 2 | 89 | 0.01 |
| | pathway | | | |
| 7 | Antigen processing | 1 | 99 | 0.01 |
| | and presentation | | | |
| 8 | Adipocytokine signaling | 1 | 71 | 0.02 |
| | pathway | | | |
| 9 | Long-term depression | 2 | 76 | 0.02 |

TABLE 4
Statistically Significant KEGG Pathways Associated with
Differentially Expressed Genes for Diabetic ApoE
null/RAGE null Relative to Diabetic ApoE null mice

| | | Number of | Number | False |
|---|---|---|---|---|
| | | differentially | of | discovery |
| | | expressed | genes in | rate |
| | | genes in | KEGG | corrected |
| | | KEGG | pathway | gamma |
| Rank | Pathway Name | pathway | on chip | p-value |
| 1 | Phosphatidylinositol | 3 | 70 | 9.7E−12 |
| | signaling system | | | |
| 2 | Wnt signaling pathway | 3 | 141 | 0.02 |
| 3 | Focal adhesion | 6 | 186 | 0.02 |
| 4 | MAPK signaling pathway | 5 | 249 | 0.02 |
| 5 | Melanoma | 2 | 70 | 0.02 |
| 6 | Gap junction | 2 | 84 | 0.02 |
| 7 | ErbB signaling pathway | 2 | 85 | 0.02 |
| 8 | TGF-beta signaling pathway | 5 | 86 | 0.02 |

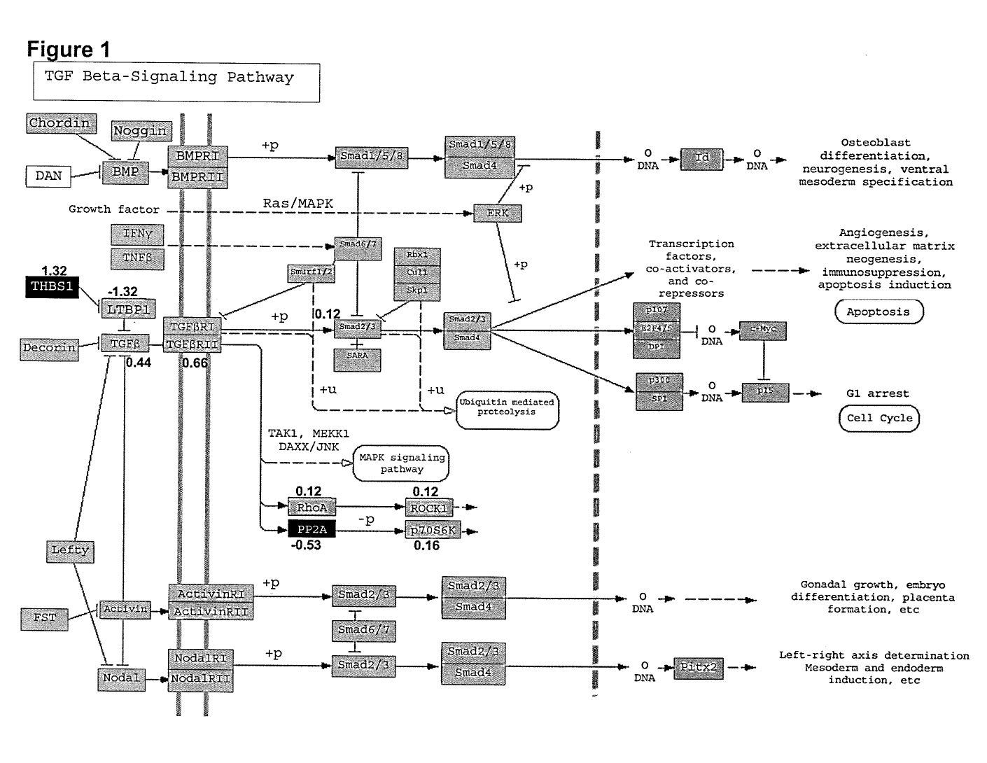

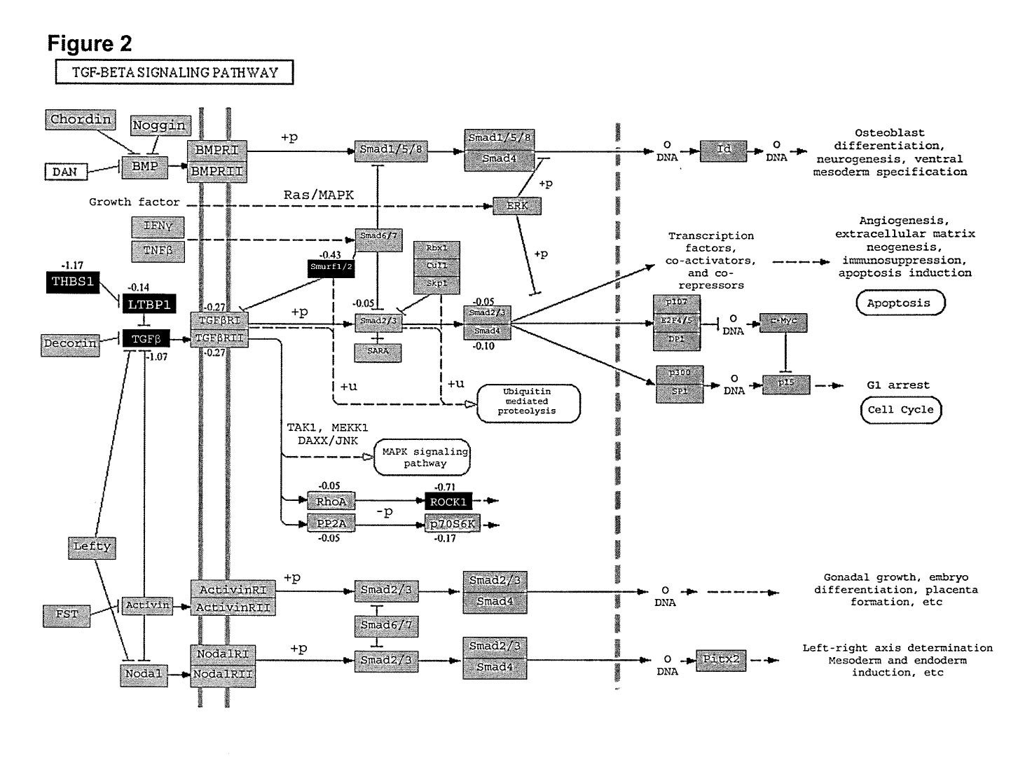

The Tgf-β pathway, with the genes that are differentially expressed indicated for the two comparisons under consideration, are given in FIGS. 1 and 2. The genes that are differentially expressed in each comparison are given in Tables 5 and 6. The genes whose perturbation factors are changed in each comparison are given in Tables 7 and 8. Perturbation factors, defined and discussed in detail by Draghici and colleagues (16), are effective log₂ fold changes which take into account both any actual change in the expression of the gene and the effect of those genes in the pathway upstream to it. Genes without a statistically significant change may still have non-zero perturbation factor.

Table 5 shows that expression of Thbs1 mRNA is increased in diabetic ApoE null mice compared to non-diabetic ApoE null mice (comparison 1). Table S6 show that expression of Thbs1 mRNA is lower in diabetic ApoE null/RAGE null mice relative to diabetic ApoE null mice (comparison 4). This finding suggests that deletion of RAGE may wholly or partially mitigate the effect of diabetes on Thbs1 up-regulation in ApoE null mice. Analysis of FIGS. 1-2 reveals that Latent transforming growth factor beta binding protein 1 (Ltbp1) is an inhibitor of Tgf-β2(17-19). Since Thbs1 inhibits the suppressive effect of Ltbp1 on activation of Tgf-β2, the results suggest that in diabetic ApoE null mice, the effect of increased Thbs1 mRNA expression is to activate Tgf-β2 protein. Similarly, FIG. 2 suggests that the reduction of Thbs1 expression in diabetic ApoE null/RAGE null mice relative to non-diabetic ApoE null mice deactivates Tgf-β2 protein. FIG. 2 and Table 6 list other genes in the Tgf-β pathway whose expression is reduced in comparison 4. Further, in addition to Thbs1 and Tgf-β2, ROCK1 is also linked to atherogenesis (20-22).

TABLE 5
Fold Changes of Differentially Expressed Tgf-β Pathway Genes
in Diabetic ApoE null Relative to Non-Diabetic ApoE null mice

| | | log₂ |
|---|---|---|
| Gene Symbol | Gene Name | FC |
| Ppp2r1b | protein phosphatase 2 (formerly 2A), | −0.53 |
| | regulatory subunit A (PR 65), beta isoform | |
| Thbs1 | thrombospondin 1 | 1.32 |

TABLE 6
Fold Changes of Differentially Expressed Tgf-β Pathway Genes in
Diabetic ApoE null/RAGE null Relative to Diabetic ApoE null mice

| Gene | | log₂ |
|---|---|---|
| Symbol | Gene Name | FC |
| Ltbp1 | latent transforming growth factor beta binding protein 1 | −1.3 |
| Rock1 | Rho-associated coiled-coil containing protein kinase 1 | −0.66 |
| Smurf2 | SMAD specific E3 ubiquitin protein ligase 2 | −0.43 |
| Tgfb2 | transforming growth factor, beta 2 | −1.1 |
| Thbs1 | thrombospondin 1 | −1.2 |

TABLE 7
Perturbation Factors of Differentially Expressed Tgf-β Pathway Genes in
Diabetic ApoE null Relative to Non-Diabetic ApoE null mice

| Gene | | | log₂ | Is Input |
|---|---|---|---|---|
| Symbol | Gene Name | Perturbation Factor | FC | Gene |
| Ltbp1 | latent transforming growth factor beta binding protein 1 | −1.32 | 0 | No |
| Ppp2ca | protein phosphatase 2 (formerly 2A), catalytic subunit, alpha | 0.12 | 0 | No |
| Ppp2cb | protein phosphatase 2 (formerly 2A), catalytic subunit, beta isoform | 0.12 | 0 | No |
| Ppp2r1a | protein phosphatase 2 (formerly 2A), regulatory subunit A (PR 65), alpha isoform | 0.12 | 0 | No |
| Ppp2r1b | protein phosphatase 2 (formerly 2A), regulatory subunit A (PR 65), beta isoform | −0.41 | −0.53 | Yes |
| Ppp2r2b | protein phosphatase 2 (formerly 2A), regulatory subunit B (PR 52), beta isoform | 0.12 | 0 | No |
| Ppp2r2c | protein phosphatase 2 (formerly 2A), regulatory subunit B (PR 52), gamma isoform | 0.12 | 0 | No |
| Ppp2r2d | protein phosphatase 2, regulatory subunit B, delta isoform | 0.12 | 0 | No |
| Rhoa | ras homolog gene family, member A | 0.12 | 0 | No |
| Rock1 | Rho-associated coiled-coil containing protein kinase 1 | 0.12 | 0 | No |
| Rock2 | Rho-associated coiled-coil containing protein kinase 2 | 0.12 | 0 | No |
| Rps6kb1 | ribosomal protein S6 kinase, polypeptide 1 | 0.16 | 0 | No |
| Rps6kb2 | ribosomal protein S6 kinase, polypeptide 2 | 0.16 | 0 | No |
| Smad2 | MAD homolog 2 (Drosophila) | 0.12 | 0 | No |
| Smad3 | MAD homolog 3 (Drosophila) | 0.12 | 0 | No |
| Smad4 | MAD homolog 4 (Drosophila) | 0.24 | 0 | No |
| Tgfb1 | transforming growth factor, beta 1 | 0.44 | 0 | No |
| Tgfb2 | transforming growth factor, beta 2 | 0.44 | 0 | No |
| Tgfb3 | transforming growth factor, beta 3 | 0.44 | 0 | No |
| Tgfbr1 | transforming growth factor, beta receptor I | 0.66 | 0 | No |
| Tgfbr2 | transforming growth factor, beta receptor II | 0.66 | 0 | No |
| Thbs1 | thrombospondin 1 | 1.32 | 1.32 | Yes |

TABLE 8
Perturbation Factors of Differentially Expressed Tgf-β Pathway Genes in Diabetic
ApoE null/RAGE null Relative to Diabetic ApoE null mice

| Gene | | Perturbation | | Is Input |
|---|---|---|---|---|
| Symbol | Gene Name | Factor | Fold change | Gene |
| Ltbp1 | latent transforming growth factor beta binding protein 1 | −0.14 | −1.31 | Yes |
| Ppp2ca | protein phosphatase 2 (formerly 2A), catalytic subunit, alpha | −0.05 | 0.00 | No |
| Ppp2cb | protein phosphatase 2 (formerly 2A), catalytic subunit, beta isoform | −0.05 | 0.00 | No |
| Ppp2r1a | protein phosphatase 2 (formerly 2A), regulatory subunit A (PR 65), alpha isoform | −0.05 | 0.00 | No |
| Ppp2r1b | protein phosphatase 2 (formerly 2A), regulatory subunit A (PR 65), beta isoform | −0.05 | 0.00 | No |
| Ppp2r2b | protein phosphatase 2 (formerly 2A), regulatory subunit B (PR 52), beta isoform | −0.05 | 0.00 | No |
| Ppp2r2c | protein phosphatase 2 (formerly 2A), regulatory subunit B (PR 52), gamma isoform | −0.05 | 0.00 | No |
| Ppp2r2d | protein phosphatase 2, regulatory subunit B, delta isoform | −0.05 | 0.00 | No |
| Rhoa | ras homolog gene family, member A | −0.05 | 0.00 | No |
| Rock1 | Rho-associated coiled-coil containing protein kinase 1 | −0.71 | −0.66 | Yes |
| Rock2 | Rho-associated coiled-coil containing protein kinase 2 | −0.05 | 0.00 | No |
| Rps6kb1 | ribosomal protein S6 kinase, polypeptide 1 | −0.17 | 0.00 | No |
| Rps6kb2 | ribosomal protein S6 kinase, polypeptide 2 | −0.17 | 0.00 | No |
| Smad2 | MAD homolog 2 (Drosophila) | −0.05 | 0.00 | No |
| Smad3 | MAD homolog 3 (Drosophila) | −0.05 | 0.00 | No |
| Smad4 | MAD homolog 4 (Drosophila) | −0.10 | 0.00 | No |
| Smurf2 | SMAD specific E3 ubiquitin protein ligase 2 | −0.43 | −0.43 | Yes |
| Tgfb1 | transforming growth factor, beta 1 | 0.05 | 0.00 | No |
| Tgfb2 | transforming growth factor, beta 2 | −1.07 | −1.12 | Yes |
| Tgfb3 | transforming growth factor, beta 3 | 0.05 | 0.00 | No |
| Tgfbr1 | transforming growth factor, beta receptor I | −0.27 | 0.00 | No |
| Tgfbr2 | transforming growth factor, beta receptor II | −0.27 | 0.00 | No |
| Thbs1 | thrombospondin 1 | −1.17 | −1.17 | Yes |

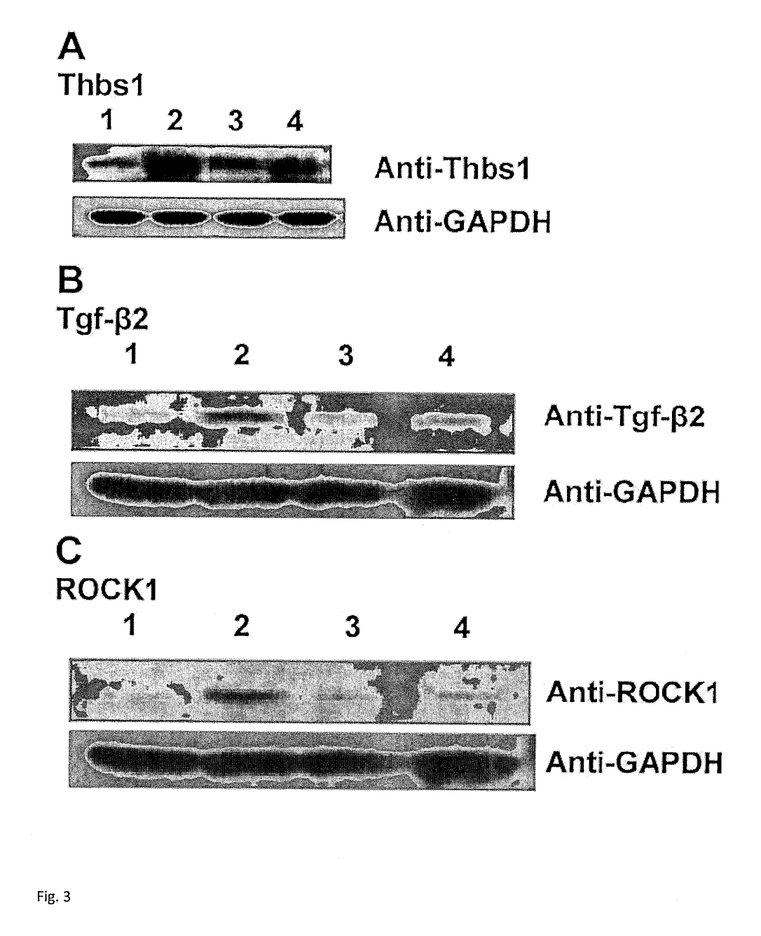

Real-time quantitative PCR followed by Western blotting was used to validate the microarray results for Thbs1, Tgf-β2, and ROCK1 by (Table 9, FIG. 3). These data reveal that diabetes increases protein levels of Thbs1, Tgf-β2 and ROCK1 in ApoE null aorta, and that particularly in the diabetic state; deletion of RAGE suppresses diabetes-linked up-regulation of Thbs1, Tgf-β2 and ROCK1 protein in ApoE null aorta.

TABLE 9
Change in mRNA Expression by both Microarray and PCR and Protein Expression (Western
Blotting) of
Key Genes in the ROCK1 Branch of the Tgf-β Pathway

| comparison # | | | | | | | | | | | | | | |
|---|---|---|---|---|---|---|---|---|---|---|---|---|---|---|
| 1 | Diabetic ApoE null relative to Non-diabetic ApoE null | | | | | | | | | | | | | |
| | Microarray | | | | | | | | | | | | | |
| | | | | | | | PCR | | | | | Western Blot | | |
| | | | | | | | ApoE null ND | ApoE null D | | | | ApoE null ND | ApoE null D | |
| | | log₂FC | FC | P(BH) | B | sig | ΔCt | ΔCt | log₂FC(95% CL) | FC(95% CL) | P | log10FC(95% CL) | FC(95% CL) | P |
| | Thbs1 | 1.32 | 2.50 | 0.22 | 1.50 | Y | 12.34(11.92, 12.75) | 11.07(10.89, 11.25) | 1.27(0.88, 1.66) | 2.41(1.84, 3.17) | 8.E−04 | 0.29(0.25, 0.33) | 2.0(1.8, 2.1) | 7.E−04 |
| | Tgf-Beta2 | 0.45 | 1.37 | 0.40 | −3.19 | N | 11.50(10.18, 12.82) | 11.02(9.82, 12.22) | 0.48(−0.89, 1.86) | 1.39(0.54, 3.62) | 0.42 | 0.12(0.03, 0.21) | 1.3(1.1, 1.6) | 0.03 |
| | ROCK1 | 0.32 | 1.25 | 0.44 | −3.55 | N | 11.52(10.73, 12.31) | 11.03(9.96, 12.11) | 0.49(−1.02, 1.53) | 1.40(0.49, 2.90) | 0.29 | 0.42(0.66, 1.08) | 2.78(4.52, 11.97) | 9.E−04 |
| 2 | Non-diabetic ApoE null/RAGE null relative to non-diabetic ApoE null | | | | | | | | | | | | | |
| | Microarray | | | | | | | | | | | | | |
| | | | | | | | PCR | | | | | Western Blot | | |
| | | | | | | | ApoE null ND | Double null ND | | | | ApoE null ND | Double null ND | |
| | | log₂FC | FC | P(BH) | B | sig | ΔCt | ΔCt | log₂FC(95% CL) | FC(95% CL) | P | log10FC(95% CL) | FC(95% CL) | P |
| | Thbs1 | 0.32 | 1.25 | 1.00 | −4.55 | N | 12.34(11.92, 12.75) | 11.69 | 0.65(−1.64, 2.92) | 1.56(1.19, | 0.37 | 0.007(−0.122, | 1.02(0.76, 1.36) | 0.87 |
| | | | | | | | | (9.26, 14.13) | | 7.57) | | 0.135) | | |
| | Tgf-Beta2 | −0.25 | 0.84 | 100 | −4.50 | N | 11.50(10.18, 12.82) | 11.57(10.51, 12.62) | −0.06(−1.34, | 0.95(0.40, 2.30) | 0.90 | −0.01(−0.02, 0.00) | 0.97(0.95, 0.00) | 0.06 |
| | | | | | | | | | 1.20) | | | | | |
| | ROCK1 | 0.24 | 1.18 | 1.00 | −4.66 | N | 11.52(10.73, 12.31) | 11.27(10.57, 11.99) | 0.24(−0.53, 1.01) | 1.18(0.69, 2.02) | 0.46 | 0.024 | 1.06(1.02, 1.09) | 0.02 |
| | | | | | | | | | | | | (0.011, 0.038) | | |
| 3 | Diabetic ApoE null/RAGE null relative to non-diabetic ApoE null/RAGE null | | | | | | | | | | | | | |
| | Microarray | | | | | | | | | | | | | |
| | | | | | | | PCR | | | | | Western Blot | | |
| | | | | | | | Double null ND | Double null D | | | | Double null ND | Double null D | |
| | | log₂FC | FC | P(BH) | B | sig | ΔCt | ΔCt | log₂FC(95% CL) | FC(95% CL) | P | log10FC(95% CL) | FC(95% CL) | P |
| | Thbs1 | 0.29 | 1.22 | 0.85 | −4.91 | N | 11.69 | 12.08(11.94, 12.22) | −0.38(−2.80, | 0.77(0.14, 4.06) | 0.56 | −0.06(−0.18, 0.06) | 0.87(0.66, | 0.22 |
| | | | | | | | (9.26, 14.13) | | 2.02) | | | | 1.51) | |
| | Tgf-Beta2 | −0.40 | 0.76 | 0.69 | −3.81 | N | 11.57(10.51, 12.62) | 12.43(11.83, 13.02) | −0.86(−1.71, −0.01) | 0.55(0.31, 1.00) | 0.05 | −0.002(0.020, .017) | 1.00(0.95, 1.040) | 0.79 |
| | ROCK1 | −1.57 | 0.34 | 0.67 | −2.58 | N | 11.27(10.57, 11.99) | 12.54(11.48, 13.60) | −1.55(−2.27, −0.25) | 0.34(0.21, 0.84) | 2.E−04 | −0.01(−0.02, 0.00) | 0.98(0.96, 1.01) | 0.11 |
| 4 | Diabetic ApoE null/RAGE null relative to diabetic ApoE null | | | | | | | | | | | | | |
| | Microarray | | | | | | | | | | | | | |
| | | | | | | | PCR | | | | | Western Blot | | |
| | | | | | | | ApoE null D | Double null D | | | | ApoE null D | Double null D | |
| | | log₂FC | FC | P(BH) | B | sig | ΔCt | ΔCt | log₂FC(95% CL) | FC(95% CL) | P | log10FC(95% CL) | FC(95% CL) | P |
| | Thbs1 | −1.17 | 0.44 | 0.06 | 0.84 | Y | 11.07(10.89, 11.25) | 12.08(11.94, 12.22) | −1.02(−1.19, −0.84) | 0.49(.56, .81) | 2.E−06 | −0.48(−0.66, −0.29) | 0.33(0.22, 0.51) | 6.E−03 |
| | Tgt-Beta2 | −1.09 | 0.47 | 0.07 | 0.36 | Y | 11.02(9.82, 12.22) | 12.43(11.83, 13.02) | −1.41(−2.54, −0.28) | 0.36(0.17, 0.82) | 0.02 | −0.14(−0.23, −0.05) | 0.72(0.59, 0.89) | 0.02 |
| | ROCK1 | −0.66 | 0.63 | 0.06 | 0.60 | Y | 11.03(9.96, 12.11) | 12.54(11.48, 13.60) | −1.50(−2.67, −0.34) | 0.35(0.16, 0.79) | 0.02 | −0.27(−0.31, −0.22) | 0.54(0.49, 0.60) | 0.001 |
| 5 | Diabetic ApoE null/RAGE null relative to non-diabetic Apo E null | | | | | | | | | | | | | |
| | Microarray | | | | | | | | | | | | | |
| | | | | | | | PCR | | | | | Western Blot | | |
| | | | | | | | ApoE null D | Double null D | | | | ApoE null D | Double null D | |
| | | log₂FC | FC | P(BH) | B | sig | ΔCt | ΔCt | log₂FC(95% CL) | FC(95% CL) | P | log10FC(95% CL) | FC(95% CL) | P |
| | Thbs1 | 0.40 | 1.32 | 0.67 | −4.75 | N | 12.34(11.92, 12.75) | 12.08(11.94, 12.22) | 0.25(−0.14, 0.65) | 1.19(0.91, 1.57) | 0.14 | −0.05(−0.12, 0.01) | 0.88(0.76, 1.02) | 0.08 |
| | Tgf-Beta2 | −0.56 | 0.68 | 0.45 | −2.22 | N | 11.50(10.18, 12.82) | 12.43(11.83, 13.02) | −0.93(−2.17, | 0.53(0.22, 1.25) | 0.11 | −0.01(−0.03, 0.00) | 0.97(0.93, 1.01) | 0.11 |
| | | | | | | | | | 0.22) | | | | | |
| | ROCK1 | −1.14 | 0.45 | 0.51 | −3.38 | N | 11.52(10.73, 12.31) | 12.54(11.48, 13.60) | −1.01(−2.06, | 0.49(0.24, 1.02) | 0.05 | 0.017(0.006, 0.029) | 1.04(1.01, 1.07) | 0.01 |
| | | | | | | | | | 0.02) | | | | | |
| 6 | Non-diabetic Apo E null/RAGE null relative to diabetic ApoE null | | | | | | | | | | | | | |
| | Microarray | | | | | | | | | | | | | |
| | | | | | | | PCR | | | | | Western Blot | | |
| | | | | | | | ApoE null D | Double null ND | | | | ApoE null D | Double null ND | |
| | | log₂FC | FC | P(BH) | B | sig | ΔCt | ΔCt | log₂FC(95% CL) | FC(95% CL) | P | log10FC(95% CL) | FC(95% CL) | P |
| | Thbs1 | −1.00 | 0.50 | 0.24 | −1.00 | N | 11.07(10.89, 11.25) | 11.69 | −0.63(−3.02, | 0.65(0.12, 3.47) | 0.38 | −0.42(−0.59, −0.24) | 0.38(0.26, 0.57) | 0.003 |
| | | | | | | | | (9.26, 14.13) | 1.77) | | | | | |
| | Tgf-Beta2 | −0.81 | 0.57 | 0.21 | 0.27 | N | 11.02(9.82, 12.22) | 11.57(10.51, 12.62) | −0.55(−1.72, | 0.68(0.30, 1.54) | 0.28 | −0.14(−0.23, −0.04) | 0.73(0.58, 0.91) | 0.02 |
| | | | | | | | | | 0.62) | | | | | |
| | ROCK1 | −0.22 | 0.86 | 0.64 | 4.82 | N | 11.03(9.96, 12.11) | 11.27(10.57, 11.99) | −0.25(−1.27, | 0.84(0.42, 1.71) | 0.55 | −0.26(−0.31, −0.21) | 0.55(0.49, 0.61) | 0.002 |
| | | | | | | | | | 0.78) | | | | | |

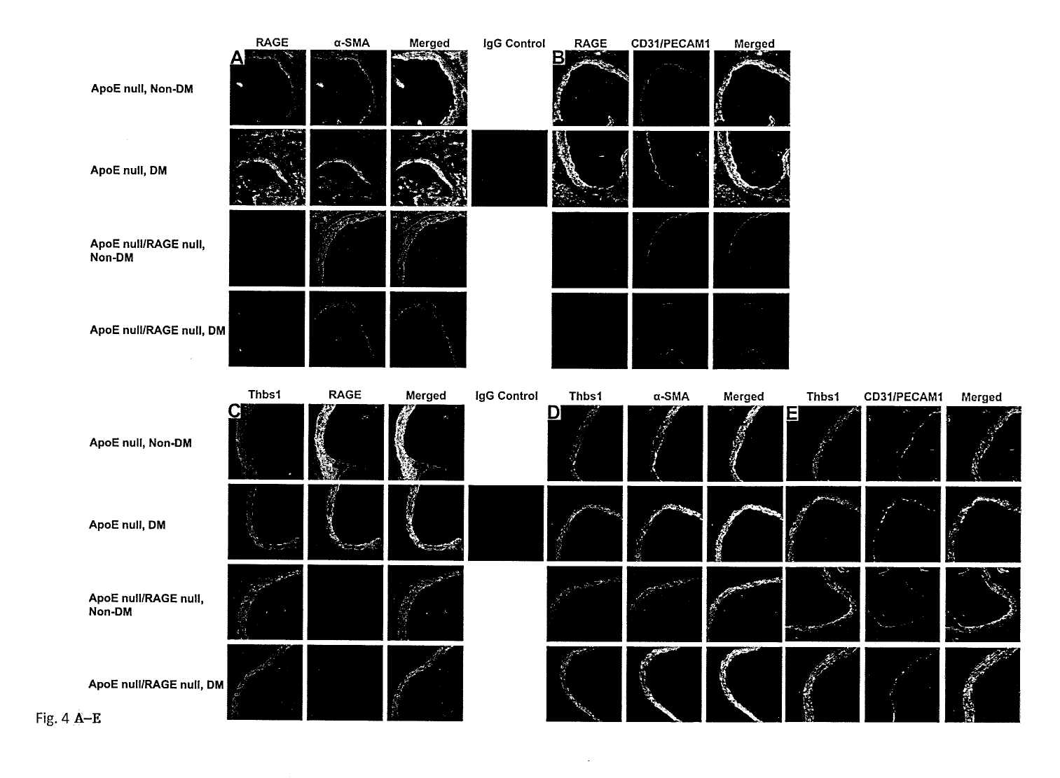

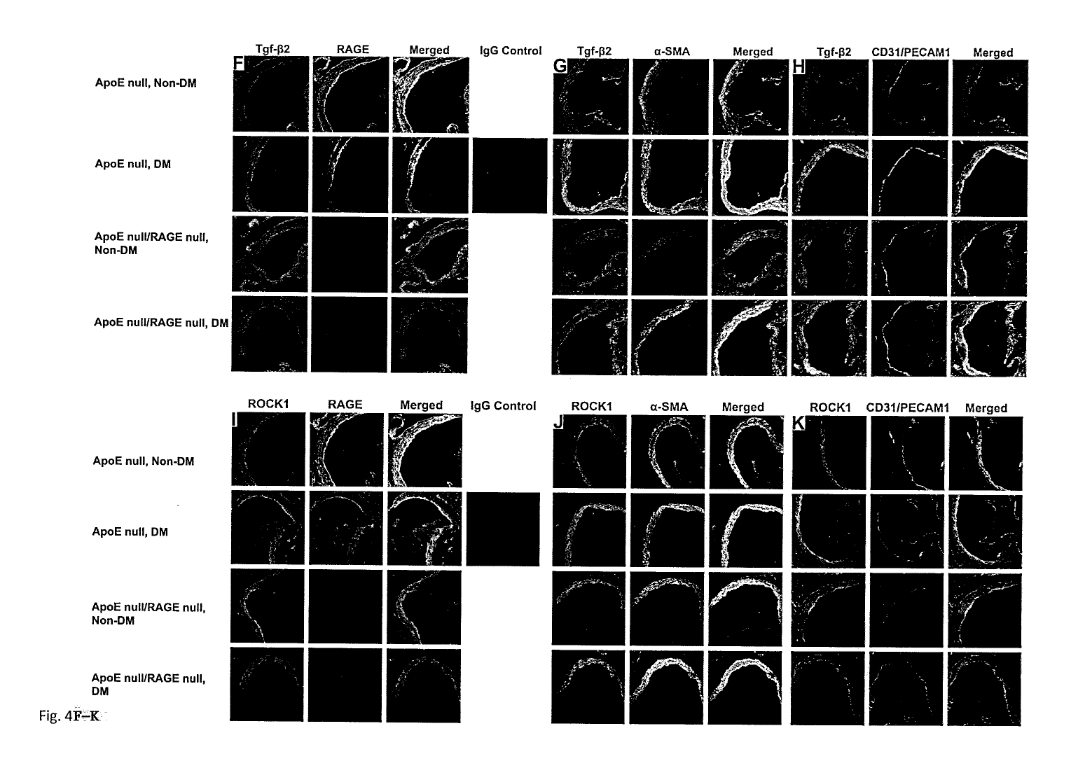

In order to identify the specific histological distribution of the key molecules identified in this model, mouse aorta sections were immunostained from the four groups of mice being studied and these sections were subjected to confocal microscopy (FIG. 4). First, the expression of RAGE was examined in the aorta of ApoE null mice at age 9 weeks (FIG. 4). RAGE is absent in the RAGE null animals, as has been observed previously (5). In both non-diabetic and diabetic ApoE null mice, RAGE (green) is expressed in SMCs (α-smooth muscle actin (red) (FIG. 4A), as indicated by the (yellow) merged images in column 3. Furthermore, RAGE (green) and CD31/PECAM1 (red) are colocalized, indicating that RAGE is expressed in the EC as well (FIG. 4B).

The cellular localization of the three key genes of the Tgf-β pathway identified in these studies was determined. FIG. 4C shows co-localization of Thbs1 with RAGE. The Thbs1 and α-SMA images merge, under all four conditions, indicating co-localization of the two proteins in the smooth muscle layer (FIG. 4D), consistent with what has been observed previously (23). However, the Thbs1 and CD31/PECAM1 images do not merge under any of the four conditions (FIG. 4E), indicating that Thbs1 is not expressed to appreciable degrees in the endothelial layer of ApoE null mice at age 9 weeks, although Thbs1 expression in ECs has been noted in other settings (24).

TGF-β2 is coexpressed with RAGE in the aorta (FIG. 4F). In all cases, TGF-β2 merges with RAGE and α-SMA or CD31/PECAM1, with the exception of CD31/PECAM1 in non-diabetic ApoE null mice, indicating that TGF-32 is expressed in SMCs in all conditions and in endothelial layers in diabetic but not non-diabetic ApoE null mice aorta (FIG. 40,H). Furthermore, ROCK1 is coexpressed with RAGE. The images of ROCK1 and RAGE merge, indicating that the two molecules are colocalized (FIG. 4T). Further, ROCK1 and α-SMA are also colocalized, indicating that ROCK1 is expressed in the smooth muscle layer (FIG. 4J). The images of ROCK1 and CD31/PECAN colocalize weakly in non-diabetic and diabetic ApoE null mice (FIG. 4K). These findings suggest that ROCK1 is predominantly expressed in the smooth muscle layer in early atherogenesis in ApoE null aorta, but previous studies have noted EC expression as well (25).

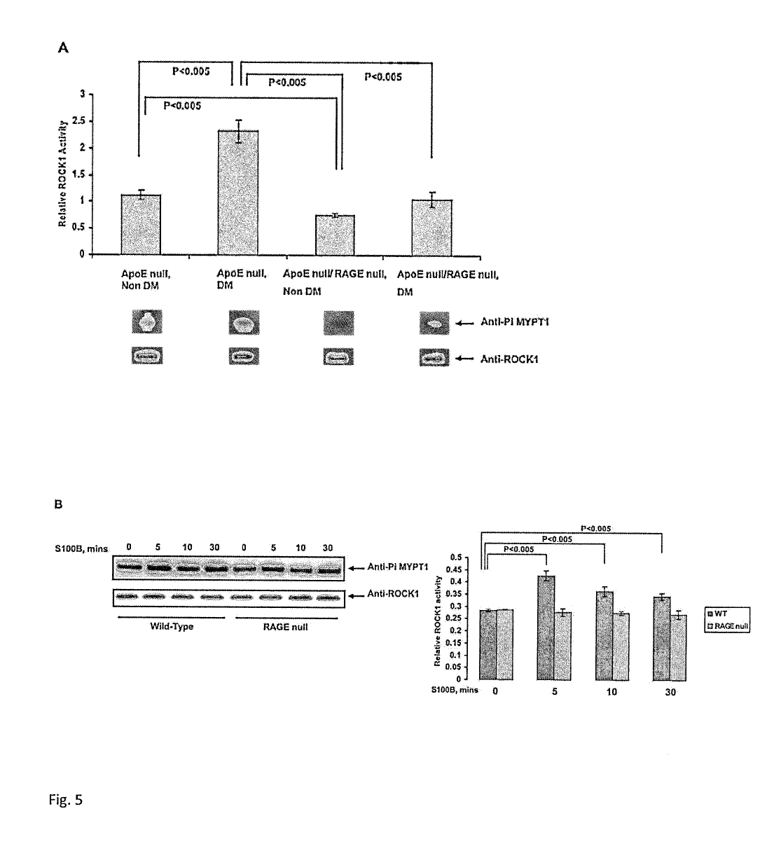

Because the analyses suggested that the ROCK1 branch of the Tgf-β pathway is importantly involved in RAGE-dependent atherogenesis, the activation state of ROCK1 in this tissue was assessed. The relative quantity of phosphorylated MYPT1/Ppp1r12a, which is directly phosphorylated by ROCK1 (26-27) was measured, and serves as a measure of the quantity of activated ROCK1. FIG. 5A shows that the extent of MYPT1/Ppp1r12a phosphorylation increases in diabetic ApoE null mouse aorta relative to non-diabetic ApoE null mice aorta, and diabetic ApoE null/RAGE null mice reveal significantly decreased MYPT1/Ppp1r12a phosphorylation vs. diabetic ApoE null mice. Furthermore, as SMCs were the primary cell type expressing ROCK1 in the aorta, SMCs were retrieved from the aortas of wild-type and RAGE null mice and treated them with RAGE ligand. Although primary aortic SMCs from wild-type mice displayed increased ROCK1 activity upon incubation with RAGE ligand, S100b, SMCs from RAGE null mice failed to increase ROCK1 activity under these conditions (FIG. 5B).

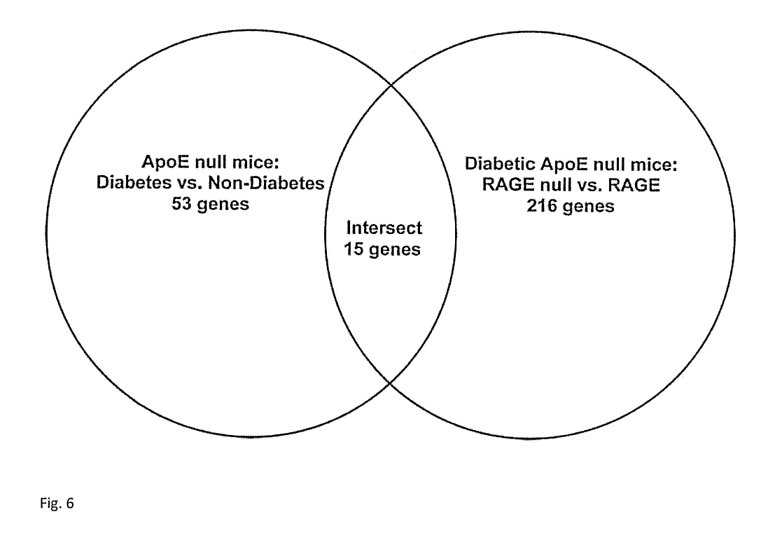

The data reveal that the observed changes in the TGFβ pathway are typical of changes in transcription associated with atherogenesis accompanying the onset of diabetes in ApoE null mice, and the effect of RAGE deletion in diabetic ApoE null mice. Table 9 provides the numbers of differentially expressed unique genes (as distinct from probesets) for each comparison that have Entrez Gene symbols and the numbers with positive and negative log fold changes. In addition, this table gives the numbers of genes resulting from Boolean operations on these genelists. Tables 11-15 give the lists of genes whose numbers are given in Table 8. FIG. 6 represents a Venn diagram showing the intersection of comparison 1, diabetic ApoE null relative to non-diabetic ApoE null, with comparison 4, diabetic ApoE null/RAGE null relative to diabetic ApoE null. Although there are 53 genes which are statistically significantly differentially expressed in diabetic ApoE null relative to the non-diabetic ApoE null state, and 216 genes which are statistically significantly differentially expressed in diabetic ApoE null/RAGE null relative to diabetic ApoE null, only 15 of these genes are statistically significantly differentially expressed in both comparisons (Tables 10 and 11 and FIG. 6). There is very little overlap of the genes which are differentially expressed both in the onset of diabetes in ApoE null mice and in the effect of RAGE deletion in diabetic ApoE null mice.

TABLE 10
Differentially Expressed Genes with Unique Entrez Gene Symbols
for Diabetic ApoE null mice vs. Non-Diabetic ApoE null mice
and for Diabetic ApoE null/RAGE null vs. Diabetic ApoE null mice,
and their Directional and Boolean Subsets

| Table | Gene set or subset | |
|---|---|---|
| S10 | Diabetic ApoE null mice vs. Non-Diabetic ApoE null mice All | 53 |
| | Diabetic ApoE null/RAGE null vs. Diabetic ApoE null mice All | 216 |
| | Diabetic ApoE null mice vs. Non-Diabetic ApoE null mice All UNION Diabetic ApoE null/RAGE null vs. Diabetic ApoE null mice All | 254 |
| | Diabetic ApoE null mice vs. Non-Diabetic ApoE null mice All INTERSECTION Diabetic ApoE null/RAGE null vs. Diabetic ApoE | 15 |
| | null mice All | |
| | Diabetic ApoE null mice vs. Non-Diabetic ApoE null mice All NOT Diabetic ApoE null/RAGE null vs. Diabetic ApoE null mice All | 38 |
| | Diabetic ApoE null/RAGE null vs. diabetic ApoE null mice All NOT Diabetic ApoE null mice vs. Non-Diabetic ApoE null mice All | 201 |
| S11 | Diabetic ApoE null mice vs. Non-Diabetic ApoE null mice Positive | 34 |
| | Diabetic ApoE null/RAGE null vs. Diabetic ApoE null mice Positive | 27 |
| | Diabetic ApoE null mice vs. non-diabetic ApoE null mice Positive UNION Diabetic ApoE null/RAGE null vs. Diabetic ApoE null | 61 |
| | mice Positive | |
| | Diabetic ApoE null mice vs. non-diabetic ApoE null mice Positive INTERSECTION Diabetic ApoE null/RAGE null vs. Diabetic ApoE | 0 |
| | null mice Positive | |
| | Diabetic ApoE null mice vs. non-diabetic ApoE null mice Positive NOT Diabetic ApoE null/RAGE null vs. Diabetic ApoE null | 34 |
| | mice Positive | |
| | Diabetic ApoE null/RAGE null vs. diabetic ApoE null mice Positive NOT Diabetic ApoE null mice vs. Non-Diabetic ApoE null | 27 |
| | mice Positive | |
| S12 | Diabetic ApoE null mice vs. Non-Diabetic ApoE null mice Negative | 19 |
| | Diabetic ApoE null/RAGE null vs. Diabetic ApoE null mice Negative | 189 |
| | Diabetic ApoE null mice vs. Non-Diabetic ApoE null mice Negative UNION Diabetic ApoE null/RAGE null vs. Diabetic ApoE null | 208 |
| | mice Positive | |
| | Diabetic ApoE null mice vs. Non-Diabetic ApoE null mice Negative INTERSECTION Diabetic ApoE null/RAGE null vs. Diabetic ApoE | 0 |
| | null mice Positive | |
| | Diabetic ApoE null mice vs. Non-Diabetic ApoE null miceNegative NOT Diabetic ApoE null/RAGE null vs. Diabetic ApoE null | 19 |
| | mice Positive | |
| | Diabetic ApoE null/RAGE null vs. diabetic ApoE null mice Positive NOT Diabetic ApoE null mice vs. Non-Diabetic ApoE null | 189 |
| | mice Negative | |
| S13 | Diabetic ApoE null mice vs. Non-Diabetic ApoE null mice Positive | 34 |
| | Diabetic ApoE null/RAGE null vs. Diabetic ApoE null mice Negative | 189 |
| | Diabetic ApoE null mice vs. Non-Diabetic ApoE null mice Positive UNION Diabetic ApoE null/RAGE null vs. Diabetic ApoE null | 210 |
| | mice Positive | |
| | Diabetic ApoE null mice vs. Non-Diabetic ApoE null mice Positive INTERSECTION Diabetic ApoE null/RAGE null vs. Diabetic ApoE | 14 |
| | null mice Positive | |
| | Diabetic ApoE null mice vs. Non-Diabetic ApoE null mice Positive NOT Diabetic ApoE null/RAGE null vs. Diabetic ApoE null | 21 |
| | mice Positive | |
| | Diabetic ApoE null/RAGE null vs. Diabetic ApoE null mice Positive NOT Diabetic ApoE null mice vs. Non-Diabetic ApoE null | 175 |
| | mice Positive | |
| S14 | Diabetic ApoE null mice vs. Non-Diabetic ApoE null mice Negative | 19 |
| | Diabetic ApoE null/RAGE null vs. Diabetic ApoE null mice Positive | 27 |
| | Diabetic ApoE null mice vs. non-diabetic ApoE null mice Negative UNION Diabetic ApoE null/RAGE null vs. Diabetic ApoE null | 45 |
| | mice Positive | |
| | Diabetic ApoE null mice vs. Non-Diabetic ApoE null mice Negative INTERSECTION Diabetic ApoE null/RAGE null vs. Diabetic ApoE | 1 |
| | null mice Positive | |
| | Diabetic ApoE null mice vs. Non-Diabetic ApoE null mice Negative NOT Diabetic ApoE null/RAGE null vs. Diabetic ApoE null | 18 |
| | mice Positive | |
| | Diabetic ApoE null/RAGE null vs. Diabetic ApoE null mice Positive NOT Diabetic ApoE null mice vs. Non-Diabetic ApoE null | 26 |
| | mice Negative | |

TABLE 11
Numbers of Differentially Expressed Genes with Unique Entrez Gene Symbols
for Diabetic ApoE null mice vs. Non-Diabetic ApoE null mice
and for Diabetic ApoE null/RAGE null vs. Diabetic ApoE null mice, and their Boolean Subsets

| | | Diabetic ApoE null mice | Diabetic ApoE null | | |
|---|---|---|---|---|---|
| | Diabetic | vs. Non-Diabetic ApoE | mice vs. Non-Diabetic | | |
| Diabetic ApoE | ApoE null/ | null mice All | ApoE null mice All | Diabetic ApoE null mice vs. | |
| null mice vs. | RAGE null | UNION Diabetic | NTERSECTION | Non-Diabetic ApoE null mice | Diabetic ApoE null/RAGE null vs. |
| Non-Diabetic | vs. Diabetic | ApoE null/RAGE null | Diabetic ApoE null/ | All NOT Diabetic ApoE null/ | Diabetic ApoE null mice All NOT |
| ApoE null | ApoE null | vs. Diabetic ApoE | RAGE null vs. Diabetic | RAGE null vs. Diabetic ApoE | Diabetic ApoE null mice vs. Non- |
| mice All | mice All | null mice All | ApoE null mice All | null mice All | Diabetic ApoE null mice All |
| Aacs | Acbd3 | Aacs | Chordc1 | Aacs | Acbd3 |
| Acaca | Acot1 | Acaca | Crem | Acaca | Acot1 |
| Acacb | Anapc1 | Acacb | Cyp26b1 | Acacb | Anapc1 |
| Acly | Anln | Acbd3 | Dnaja1 | Acly | Anln |
| Acss2 | Arf3 | Acly | Erdr1 | Acss2 | Arf3 |
| Agbl3 | Arfip1 | Acot1 | Golt1b | Agbl3 | Arfip1 |
| Anxa3 | Armc8 | Acss2 | Hsp110 | Anxa3 | Armc8 |
| Chordc1 | Armcx3 | Agbl3 | Hspa1b | Ctps | Armcx3 |
| Crem | Atp6v1d | Anapc1 | Rybp | Eef1e1 | Atp6v1d |
| Ctps | B3gaint2 | Anln | S100a4 | Emp1 | B3gaint2 |
| Cyp26b1 | Bcas3 | Anxa3 | Slc7a1 | Fasn | Bcas3 |
| Dnaja1 | Bcl6 | Arf3 | Thbs1 | Figf | Bcl6 |
| Eef1e1 | Bdh1 | Arfip1 | Tparl | Fmo5 | Bdh1 |
| Emp1 | Bnip2 | Armc8 | Tshz3 | Fn1 | Bnip2 |
| Erdr1 | Btaf1 | Armcx3 | Tspan6 | H2-Q10 | Btaf1 |
| Fasn | Bxdc2 | Atp6v1d | | Hexim1 | Bxdc2 |
| Figf | Bysl | B3gaint2 | | Ifitm2 | Bysl |
| Fmo5 | C1galt1 | Bcas3 | | Kpna2 | C1galt1 |
| Fn1 | C1qtnt2 | Bcl6 | | Kras | C1qtnf2 |
| Golt1b | Cacna2d1 | Bdh1 | | Lmnb2 | Cacna2d1 |
| H2-Q10 | Cav3 | Bnip2 | | Mod1 | Cav3 |
| Hexim1 | Cbx5 | Btaf1 | | Mtmr11 | Cbx5 |
| Hsp110 | Ccm4l | Bxdc2 | | Mtmr4 | Ccm4l |
| Hspa1b | Cdr2 | Bysl | | Nudt18 | Cdr2 |
| Ifitm2 | Chd1 | C1galt1 | | Ppp2r1b | Chd1 |
| Kpna2 | Chd2 | C1qtnf2 | | Pvt1 | Chd2 |
| Kras | Chordc1 | Cacna2d1 | | Pygl | Chma1 |
| Lmnb2 | Chrna1 | Cav3 | | Rbm8a | Chsy1 |
| Mod1 | Chsy1 | Cbx5 | | Reln | Ckap4 |
| Mtmr11 | Ckap4 | Ccrn4l | | Sfpq | Cnn3 |
| Mtmr4 | Cnn3 | Cdr2 | | Sfrs3 | Col5a2 |
| Nudt18 | Col5a2 | Chd1 | | Slc1a5 | Copz2 |
| Ppp2r1b | Copz2 | Chd2 | | Slc25a1 | Creb3l2 |
| Pvt1 | Creb3l2 | Chordc1 | | Thrsp | Crispld1 |
| Pygl | Crem | Chma1 | | Tkt | Cthrc1 |
| Rbm8a | Crispld1 | Chsy1 | | Tm4sf1 | Dap |
| Reln | Cthrc1 | Ckap4 | | Ube1l2 | Dapk2 |
| Rybp | Cyp26b1 | Cnn3 | | Wwc2 | Dgkg |
| S100a4 | Dap | Col5a2 | | | Dock7 |
| Sfpq | Dapk2 | Copz2 | | | Dscr1l1 |
| Sfrs3 | Dgkg | Creb3l2 | | | Dusp12 |
| Slc1a5 | Dnaja1 | Crem | | | Dynll1 |
| Slc25a1 | Dock7 | Crispld1 | | | Efhc2 |
| Slc7a1 | Dscr1l1 | Cthrc1 | | | Eln |
| Thbs1 | Dusp12 | Ctps | | | Emb |
| Thrsp | Dynll1 | Cyp26b1 | | | Eml5 |
| Tkt | Efhc2 | Dap | | | Emp2 |
| Tm4sf1 | Eln | Dapk2 | | | Emp3 |
| Tparl | Emb | Dgkg | | | Enah |
| Tshz3 | Eml5 | Dnaja1 | | | Endod1 |
| Tspan6 | Emp2 | Dock7 | | | Ereg |
| Ube1l2 | Emp3 | Dscr1l1 | | | Ero1l |
| Wwc2 | Enah | Dusp12 | | | Ext1 |
| | Endod1 | Dynll1 | | | Fgt1 |
| | Erdr1 | Eef1e1 | | | Fjx1 |
| | Ereg | Efhc2 | | | Fkbp14 |
| | Ero1l | Eln | | | Fryl |
| | Ext1 | Emb | | | Gal3st4 |
| | Fgf1 | Eml5 | | | Ggh |
| | Fjx1 | Emp1 | | | Glo1 |
| | Fkbp14 | Emp2 | | | Glp1r |
| | Fryl | Emp3 | | | Gm347 |
| | Gal3st4 | Enah | | | Gm70 |
| | Ggh | Endod1 | | | Gmds |
| | Glo1 | Erdr1 | | | Gne |
| | Glp1r | Ereg | | | Gpatc2 |
| | Gm347 | Ero1l | | | Gpr125 |
| | Gm70 | Ext1 | | | Gpr177 |
| | Gmds | Fasn | | | Grina |
| | Gne | Fgf1 | | | H6pd |
| | Golt1b | Figf | | | Hbegf |
| | Gpatc2 | Fjx1 | | | Hcfc2 |
| | Gpr125 | Fkbp14 | | | Hdac9 |
| | Gpr177 | Fmo5 | | | Hdlbp |
| | Grina | Fn1 | | | Hisppd2a |
| | H6pd | Fryl | | | Hrasls |
| | Hbegf | Gal3st4 | | | Hrb |
| | Hcfc2 | Ggh | | | Hspa1a |
| | Hdac9 | Glo1 | | | Hspb2 |
| | Hdlbp | Glp1r | | | Htra1 |
| | Hisppd2a | Gm347 | | | Il17b |
| | Hrasls | Gm70 | | | Itga9 |
| | Hrb | Gmds | | | Itgb1bp1 |
| | Hsp110 | Gne | | | Itpr1 |
| | Hspa1a | Golt1b | | | Kcnab1 |
| | Hspa1b | Gpatc2 | | | Kdelr2 |
| | Hspb2 | Gpr125 | | | Kdelr3 |
| | Htra1 | Gpr177 | | | Kpna1 |
| | Il17b | Grina | | | Ky |
| | Itga9 | H2-Q10 | | | Lancl3 |
| | Itgb1bp1 | H6pd | | | Lasp1 |
| | Itpr1 | Hbegf | | | Lats2 |
| | Kcnab1 | Hcfc2 | | | Lbh |
| | Kdelr2 | Hdac9 | | | Leprel1 |
| | Kdelr3 | Hdlbp | | | Lox |
| | Kpna1 | Hexim1 | | | Lpp |
| | Ky | Hisppd2a | | | Lrrc61 |
| | Lancl3 | Hrasls | | | Lrrfip1 |
| | Lasp1 | Hrb | | | Ltbp1 |
| | Lats2 | Hsp110 | | | Luzp1 |
| | Lbh | Hspa1a | | | Maged2 |
| | Leprel1 | Hspa1b | | | Mapk6 |
| | Lox | Hspb2 | | | Mfap5 |
| | Lpp | Htra1 | | | Mgat5 |
| | Lrrc61 | Ifitm2 | | | Mobk1b |
| | Lrrfip1 | Il17b | | | Mrpl15 |
| | Ltbp1 | Itga9 | | | Mtdh |
| | Luzp1 | Itgb1bp1 | | | Myh10 |
| | Maged2 | Itpr1 | | | Ndel1 |
| | Mapk6 | Kcnab1 | | | Nedd9 |
| | Mfap5 | Kdelr2 | | | Ngp |
| | Mgat5 | Kdelr3 | | | Nhlrc2 |
| | Mobk1b | Kpna1 | | | Nrk |
| | Mrpl15 | Kpna2 | | | Ntf3 |
| | Mtdh | Kras | | | Olfml2b |
| | Myh10 | Ky | | | P4ha1 |
| | Ndel1 | Lancl3 | | | Pank2 |
| | Nedd9 | Lasp1 | | | Pbef1 |
| | Ngp | Lats2 | | | Pcgf5 |
| | Nhlrc2 | Lbh | | | Pdgfd |
| | Nrk | Leprel1 | | | Pdgfrl |
| | Ntf3 | Lmnb2 | | | Pdlim1 |
| | Olfml2b | Lox | | | Pdlim5 |
| | P4ha1 | Lpp | | | Pgcp |
| | Pank2 | Lrrc61 | | | Phtf2 |
| | Pbet1 | Lrrfip1 | | | Pigm |
| | Pcgf5 | Ltbp1 | | | Pip5k1a |
| | Pdgfd | Luzp1 | | | Plaur |
| | Pdgfrl | Maged2 | | | Plekha1 |
| | Pdlim1 | Mapk6 | | | Plod2 |
| | Pdlim5 | Mfap5 | | | Pols |
| | Pgcp | Mgat5 | | | Prlr |
| | Phtf2 | Mobk1b | | | Prodh |
| | Pigm | Mod1 | | | Psip1 |
| | Pip5k1a | Mrpl15 | | | Ptprz1 |
| | Plaur | Mtdh | | | Pycr1 |
| | Plekha1 | Mtmr11 | | | Rab23 |
| | Plod2 | Mtmr4 | | | Rab31 |
| | Pols | Myh10 | | | Rabep1 |
| | Prlr | Ndel1 | | | Ramp1 |
| | Prodh | Nedd9 | | | Rarres2 |
| | Psip1 | Ngp | | | Rassf8 |
| | Ptprz1 | Nhlrc2 | | | Rbbp9 |
| | Pycr1 | Nrk | | | Reck |
| | Rab23 | Ntf3 | | | Rnd1 |
| | Rab31 | Nudt18 | | | Rock1 |
| | Rabep1 | Olfml2b | | | Sacs |
| | Ramp1 | P4ha1 | | | Scx |
| | Rarres2 | Pank2 | | | Sec22l1 |
| | Rassf8 | Pbef1 | | | Sec23a |
| | Rbbp9 | Pcgf5 | | | Sec24a |
| | Reck | Pdgfd | | | Sema3c |
| | Rnd1 | Pdgfrl | | | Serpine1 |
| | Rock1 | Pdlim1 | | | Sfrp2 |
| | Rybp | Pdlim5 | | | Sgcb |
| | S100a4 | Pgcp | | | Sgce |
| | Sacs | Phtf2 | | | Sgcg |
| | Scx | Pigm | | | Sgol2 |
| | Sec22l1 | Pip5k1a | | | Sh3bgr |
| | Sec23a | Plaur | | | Sh3gl2 |
| | Sec24a | Plekha1 | | | Six5 |
| | Sema3c | Plod2 | | | Slc9a2 |
| | Serpine1 | Pols | | | Smurf2 |
| | Sfrp2 | Ppp2r1b | | | Sorbs1 |
| | Sgcb | Prlr | | | Sord |
| | Sgce | Prodh | | | Srm |
| | Sgcg | Psip1 | | | Srprb |
| | Sgol2 | Ptprz1 | | | St8sia6 |
| | Sh3bgr | Pvt1 | | | Stch |
| | Sh3gl2 | Pycr1 | | | Stk25 |
| | Six5 | Pygl | | | Stk38l |
| | Slc7a1 | Rab23 | | | Stt3a |
| | Slc9a2 | Rab31 | | | Sulf1 |
| | Smurf2 | Rabep1 | | | Surb7 |
| | Sorbs1 | Ramp1 | | | Sync |
| | Sord | Rarres2 | | | Tdrd3 |
| | Srm | Rassf8 | | | Tgfb2 |
| | Srprb | Rbbp9 | | | Timp4 |
| | St8sia6 | Rbm8a | | | Tmem39a |
| | Stch | Reck | | | Tmem45a |
| | Stk25 | Rein | | | Tmem68 |
| | Stk38l | Rnd1 | | | Tmtc3 |
| | Stt3a | Rock1 | | | Tnfaip1 |
| | Sulf1 | Rybp | | | Tnfrsf11a |
| | Surb7 | S100a4 | | | Tnfrsf12a |
| | Sync | Sacs | | | Tnrc15 |
| | Tdrd3 | Scx | | | Trp53inp2 |
| | Tgfb2 | Sec22l1 | | | Tspan2 |
| | Thbs1 | Sec23a | | | Ube2i |
| | Timp4 | Sec24a | | | Unc5d |
| | Tmem39a | Sema3c | | | Vkorc1 |
| | Tmem45a | Serpine1 | | | Wif1 |
| | Tmem68 | Sfpq | | | Wisp2 |
| | Tmtc3 | Sfrp2 | | | Wtip |
| | Tnfaip1 | Sfrs3 | | | Xdh |
| | Tnfrsf11a | Sgcb | | | Yipf5 |
| | Tnfrsf12a | Sgce | | | Zbtb41 |
| | Tnrc15 | Sgcg | | | Zcchc10 |
| | Tparl | Sgol2 | | | Zfp148 |
| | Trp53inp2 | Sh3bgr | | | Zfp451 |
| | Tshz3 | Sh3gl2 | | | Zfp9 |
| | Tspan2 | Six5 | | | |
| | Tspan6 | Slc1a5 | | | |
| | Ube2i | Slc25a1 | | | |
| | Unc5d | Slc7a1 | | | |
| | Vkorc1 | Slc9a2 | | | |
| | Wif1 | Smurf2 | | | |
| | Wisp2 | Sorbs1 | | | |
| | Wtip | Sord | | | |
| | Xdh | Srm | | | |
| | Yipf5 | Srprb | | | |
| | Zbtb41 | St8sia6 | | | |
| | Zcchc10 | Stch | | | |
| | Zfp148 | Stk25 | | | |
| | Zfp451 | Stk38l | | | |
| | Zfp9 | Stt3a | | | |
| | | Sulf1 | | | |
| | | Surb7 | | | |
| | | Sync | | | |
| | | Tdrd3 | | | |
| | | Tgfb2 | | | |
| | | Thbs1 | | | |
| | | Thrsp | | | |
| | | Timp4 | | | |
| | | Tkt | | | |
| | | Tm4sf1 | | | |
| | | Tmem39a | | | |
| | | Tmem45a | | | |
| | | Tmem68 | | | |
| | | Tmtc3 | | | |
| | | Tnfaip1 | | | |
| | | Tnfrsf11a | | | |
| | | Tnfrsf12a | | | |
| | | Tnrc15 | | | |
| | | Tparl | | | |
| | | Trp53inp2 | | | |
| | | Tshz3 | | | |
| | | Tspan2 | | | |
| | | Tspan6 | | | |
| | | Ube1l2 | | | |
| | | Ube2i | | | |
| | | Unc5d | | | |
| | | Vkorc1 | | | |
| | | Wif1 | | | |
| | | Wisp2 | | | |
| | | Wtip | | | |
| | | Wwc2 | | | |
| | | Xdh | | | |
| | | Yipf5 | | | |
| | | Zbtb41 | | | |
| | | Zcchc10 | | | |
| | | Zfb148 | | | |
| | | Zfb451 | | | |
| | | Zfp9 | | | |
| | | Xdh | | | |

TABLE 12
Numbers of Differentially Expressed Genes with Unique Entrez Gene Symbols
for Diabetic ApoE null mice vs. Non-Diabetic ApoE null mice
and for Diabetic ApoE null/RAGE null vs. Diabetic ApoE null mice
both with Positive log2 Fold Change and their Boolean Subsets

| Diabetic | | | | | |
|---|---|---|---|---|---|
| ApoE null | | | Diabetic ApoE null mice | | Diabetic ApoE null/ |
| mice vs. | Diabetic | | vs. Non-Diabetic ApoE | Diabetic ApoE null mice | RAGE null vs. Diabetic |
| Non- | ApoE null/ | Diabetic ApoE null mice vs. | null mice Positive | vs. Non-Diabetic ApoE | ApoE null mice Positive |
| Diabetic | RAGE null vs. | Non-Diabetic ApoE null mice | INTERSECTION | null mice Positive NOT | NOT Diabetic ApoE |
| ApoE null | Diabetic | Positive UNION Diabetic ApoE null/ | Diabetic ApoE null/ | Diabetic ApoE null/ | null mice vs. Non- |
| mice | ApoE null | RAGE null vs. Diabetic ApoE null mice | RAGE null vs. Diabetic | RAGE null vs. Diabetic | Diabetic ApoE null mice |
| Positive | mice Positive | Positive | ApoE null mice Positive | ApoE null mice Positive | Positive |
| Anxa3 | Acot1 | Acot1 | | Anxa3 | Acot1 |
| Chordc1 | Atp6v1d | Anxa3 | | Chordc1 | Atp6v1d |
| Crem | Bcas3 | Atp6v1d | | Crem | Bcas3 |
| Ctps | Ccrn4l | Bcas3 | | Ctps | Ccrn4l |
| Cyp26b1 | Chd1 | Ccrn4l | | Cyp26b1 | Chd1 |
| Dnaja1 | Chd2 | Chd1 | | Dnaja1 | Chd2 |
| Eef1e1 | Eml5 | Chd2 | | Eef1e1 | Eml5 |
| Emp1 | Erdr1 | Chordc1 | | Emp1 | Erdr1 |
| Figf | Glo1 | Crem | | Figf | Glo1 |
| Fn1 | Glp1r | Ctps | | Fn1 | Glp1r |
| Golt1b | Gpatc2 | Cyp26b1 | | Golt1b | Gpatc2 |
| Hexim1 | Grina | Dnaja1 | | Hexim1 | Grina |
| Hsp110 | H6pd | Eef1e1 | | Hsp110 | H6pd |
| Hspa1b | Mrpl15 | Eml5 | | Hspa1b | Mrpl15 |
| Ifitm2 | Ngp | Emp1 | | Ifitm2 | Ngp |
| Kpna2 | Pbef1 | Erdr1 | | Kpna2 | Pbef1 |
| Kras | Prlr | Figf | | Kras | Prlr |
| Lmnb2 | Prodh | Fn1 | | Lmnb2 | Prodh |
| Mtmr4 | Sgol2 | Glo1 | | Mtmr4 | Sgol2 |
| Pvt1 | Sh3gl2 | Glp1r | | Pvt1 | Sh3gl2 |
| Rbm8a | Sorbs1 | Golt1b | | Rbm8a | Sorbs1 |
| Reln | Sord | Gpatc2 | | Reln | Sord |
| Rybp | St8sia6 | Grina | | Rybp | St8sia6 |
| S100a4 | Timp4 | H6pd | | S100a4 | Timp4 |
| Sfpq | Trp53inp2 | Hexim1 | | Sfpq | Trp53inp2 |
| Sfrs3 | Ube2i | Hsp110 | | Sfrs3 | Ube2i |
| Slc7a1 | Xdh | Hspa1b | | Slc7a1 | Xdh |
| Thbs1 | | Ifitm2 | | Thbs1 | |
| Tm4sf1 | | Kpna2 | | Tm4sf1 | |
| Tparl | | Kras | | Tparl | |
| Tshz3 | | Lmnb2 | | Tshz3 | |
| Tspan6 | | Mrpl15 | | Tspan6 | |
| Ube1l2 | | Mtmr4 | | Ube1l2 | |
| Wwc2 | | Ngp | | Wwc2 | |
| | | Pbef1 | | | |
| | | Prlr | | | |
| | | Prodh | | | |
| | | Pvt1 | | | |
| | | Rbm8a | | | |
| | | Reln | | | |
| | | Rybp | | | |
| | | S100a4 | | | |
| | | Sfpq | | | |
| | | Sfrs3 | | | |
| | | Sgol2 | | | |
| | | Sh3gl2 | | | |
| | | Slc7a1 | | | |
| | | Sorbs1 | | | |
| | | Sord | | | |
| | | St8sia6 | | | |
| | | Thbs1 | | | |
| | | Timp4 | | | |
| | | Tm4sf1 | | | |
| | | Tparl | | | |
| | | Trp53inp2 | | | |
| | | Tshz3 | | | |
| | | Tspan6 | | | |
| | | Ube1l2 | | | |
| | | Ube2i | | | |
| | | Wwc2 | | | |
| | | Xdh | | | |

TABLE 13
Numbers of Differentially Expressed Genes with Unique Entrez Gene
Symbols for Diabetic ApoE null mice vs. Non-Diabetic ApoE null mice
and for Diabetic ApoE null/RAGE null vs. Diabetic ApoE null mice
both with Negative log2 Fold Change and their Boolean Subsets

| | | | Diabetic ApoE null | | Diabetic |
|---|---|---|---|---|---|
| | | | mice vs. Non-Diabetic | | ApoE null/ |
| | | Diabetic ApoE null mice | ApoE null mice | | RAGE null vs. |
| | | vs. Non-Diabetic | Negative | Diabetic ApoE null mice | Diabetic ApoE |
| | | ApoE null mice | INTERSECTION | vs. Non-Diabetic ApoE | null mice Negative |
| Diabetic ApoE null | Diabetic ApoE null/ | Negative UNION Diabetic | Diabetic ApoE null/ | null mice Negative NOT | NOT Diabetic |
| mice vs. Non- | RAGE null vs. | ApoE null/RAGE null vs. | RAGE null vs. Diabetic | Diabetic ApoE null/ | ApoE null mice vs. |
| Diabetic ApoE null | Diabetic ApoE null | Diabetic ApoE null mice | ApoE null mice | RAGE null vs. Diabetic | Non-Diabetic ApoE null |
| mice Negative | mice Negative | Negative | Negative | ApoE null mice Negative | mice Negative |
| Aacs | Acbd3 | Aacs | No Entries | Aacs | Acbd3 |
| Acaca | Anapc1 | Acaca | | Acaca | Anapc1 |
| Acacb | Anln | Acacb | | Acacb | Anln |
| Acly | Arf3 | Acly | | Acly | Arf3 |
| Acss2 | Arfip1 | Acss2 | | Acss2 | Arfip1 |
| Agbl3 | Armc8 | Agbl3 | | Agbl3 | Armc8 |
| Erdr1 | Armcx3 | Erdr1 | | Erdr1 | Armcx3 |
| Fasn | B3galnt2 | Fasn | | Fasn | B3galnt2 |
| Fmo5 | Bcl6 | Fmo5 | | Fmo5 | Bcl6 |
| H2-Q10 | Bdh1 | H2-Q10 | | H2-Q10 | Bdh1 |
| Mod1 | Bnip2 | Mod1 | | Mod1 | Bnip2 |
| Mtmr11 | Btaf1 | Mtmr11 | | Mtmr11 | Btaf1 |
| Nudt18 | Bxdc2 | Nudt18 | | Nudt18 | Bxdc2 |
| Ppp2r1b | Bysl | Ppp2r1b | | Ppp2r1b | Bysl |
| Pygl | C1galt1 | Pygl | | Pygl | C1galt1 |
| Slc1a5 | C1qtnf2 | Slc1a5 | | Slc1a5 | C1qtnf2 |
| Slc25a1 | Cacna2d1 | Slc25a1 | | Slc25a1 | Cacna2d1 |
| Thrsp | Cav3 | Thrsp | | Thrsp | Cav3 |
| Tkt | Cbx5 | Tkt | | Tkt | Cbx5 |
| | Cdr2 | Acbd3 | | | Cdr2 |
| | Chordc1 | Anapc1 | | | Chordc1 |
| | Chrna1 | Anln | | | Chrna1 |
| | Chsy1 | Arf3 | | | Chsy1 |
| | Ckap4 | Arfip1 | | | Ckap4 |
| | Cnn3 | Armc8 | | | Cnn3 |
| | Col5a2 | Armcx3 | | | Col5a2 |
| | Copz2 | B3galnt2 | | | Copz2 |
| | Creb3l2 | Bcl6 | | | Creb3l2 |
| | Crem | Bdh1 | | | Crem |
| | Crispld1 | Bnip2 | | | Crispld1 |
| | Cthrc1 | Btaf1 | | | Cthrc1 |
| | Cyp26b1 | Bxdc2 | | | Cyp26b1 |
| | Dap | Bysl | | | Dap |
| | Dapk2 | C1galt1 | | | Dapk2 |
| | Dgkg | C1qtnf2 | | | Dgkg |
| | Dnaja1 | Cacna2d1 | | | Dnaja1 |
| | Dock7 | Cav3 | | | Dock7 |
| | Dscr1l1 | Cbx5 | | | Dscr1l1 |
| | Dusp12 | Cdr2 | | | Dusp12 |
| | Dynll1 | Chordc1 | | | Dynll1 |
| | Efhc2 | Chrna1 | | | Efhc2 |
| | Eln | Chsy1 | | | Eln |
| | Emb | Ckap4 | | | Emb |
| | Emp2 | Cnn3 | | | Emp2 |
| | Emp3 | Col5a2 | | | Emp3 |
| | Enah | Copz2 | | | Enah |
| | Endod1 | Creb3l2 | | | Endod1 |
| | Ereg | Crem | | | Ereg |
| | Ero1l | Crispld1 | | | Ero1l |
| | Ext1 | Cthrc1 | | | Ext1 |
| | Fgf1 | Cyp26b1 | | | Fgf1 |
| | Fjx1 | Dap | | | Fjx1 |
| | Fkbp14 | Dapk2 | | | Fkbp14 |
| | Fryl | Dgkg | | | Fryl |
| | Gal3st4 | Dnaja1 | | | Gal3st4 |
| | Ggh | Dock7 | | | Ggh |
| | Gm347 | Dscr1l1 | | | Gm347 |
| | Gm70 | Dusp12 | | | Gm70 |
| | Gmds | Dynll1 | | | Gmds |
| | Gne | Efhc2 | | | Gne |
| | Golt1b | Eln | | | Golt1b |
| | Gpr125 | Emb | | | Gpr125 |
| | Gpr177 | Emp2 | | | Gpr177 |
| | Hbegf | Emp3 | | | Hbegf |
| | Hcfc2 | Enah | | | Hcfc2 |
| | Hdac9 | Endod1 | | | Hdac9 |
| | Hdlbp | Ereg | | | Hdlbp |
| | Hisppd2a | Ero1l | | | Hisppd2a |
| | Hrasls | Ext1 | | | Hrasls |
| | Hrb | Fgf1 | | | Hrb |
| | Hsp110 | Fjx1 | | | Hsp110 |
| | Hspa1a | Fkbp14 | | | Hspa1a |
| | Hspa1b | Fryl | | | Hspa1b |
| | Hspb2 | Gal3st4 | | | Hspb2 |
| | Htra1 | Ggh | | | Htra1 |
| | Il17b | Gm347 | | | Il17b |
| | Itga9 | Gm70 | | | Itga9 |
| | Itgb1bp1 | Gmds | | | Itgb1bp1 |
| | Itpr1 | Gne | | | Itpr1 |
| | Kcnab1 | Golt1b | | | Kcnab1 |
| | Kdelr2 | Gpr125 | | | Kdelr2 |
| | Kdelr3 | Gpr177 | | | Kdelr3 |
| | Kpna1 | Hbegf | | | Kpna1 |
| | Ky | Hcfc2 | | | Ky |
| | Lancl3 | Hdac9 | | | Lancl3 |
| | Lasp1 | Hdlbp | | | Lasp1 |
| | Lats2 | Hisppd2a | | | Lats2 |
| | Lbh | Hrasls | | | Lbh |
| | Leprel1 | Hrb | | | Leprel1 |
| | Lox | Hsp110 | | | Lox |
| | Lpp | Hspa1a | | | Lpp |
| | Lrrc61 | Hspa1b | | | Lrrc61 |
| | Lrrfip1 | Hspb2 | | | Lrrfip1 |
| | Ltbp1 | Htra1 | | | Ltbp1 |
| | Luzp1 | Il17b | | | Luzp1 |
| | Maged2 | Itga9 | | | Maged2 |
| | Mapk6 | Itgb1bp1 | | | Mapk6 |
| | Mfap5 | Itpr1 | | | Mfap5 |
| | Mgat5 | Kcnab1 | | | Mgat5 |
| | Mobk1b | Kdelr2 | | | Mobk1b |
| | Mtdh | Kdelr3 | | | Mtdh |
| | Myh10 | Kpna1 | | | Myh10 |
| | Ndel1 | Ky | | | Ndel1 |
| | Nedd9 | Lancl3 | | | Nedd9 |
| | Nhlrc2 | Lasp1 | | | Nhlrc2 |
| | Nrk | Lats2 | | | Nrk |
| | Ntf3 | Lbh | | | Ntf3 |
| | Olfml2b | Leprel1 | | | Olfml2b |
| | P4ha1 | Lox | | | P4ha1 |
| | Pank2 | Lpp | | | Pank2 |
| | Pcgf5 | Lrrc61 | | | Pcgf5 |
| | Pdgfd | Lrrfip1 | | | Pdgfd |
| | Pdgfrl | Ltbp1 | | | Pdgfrl |
| | Pdlim1 | Luzp1 | | | Pdlim1 |
| | Pdlim5 | Maged2 | | | Pdlim5 |
| | Pgcp | Mapk6 | | | Pgcp |
| | Phtf2 | Mfap5 | | | Phtf2 |
| | Pigm | Mgat5 | | | Pigm |
| | Pip5k1a | Mobk1b | | | Pip5k1a |
| | Plaur | Mtdh | | | Plaur |
| | Plekha1 | Myh10 | | | Plekha1 |
| | Plod2 | Ndel1 | | | Plod2 |
| | Pols | Nedd9 | | | Pols |
| | Psip1 | Nhlrc2 | | | Psip1 |
| | Ptprz1 | Nrk | | | Ptprz1 |
| | Pycr1 | Ntf3 | | | Pycr1 |
| | Rab23 | Olfml2b | | | Rab23 |
| | Rab31 | P4ha1 | | | Rab31 |
| | Rabep1 | Pank2 | | | Rabep1 |
| | Ramp1 | Pcgf5 | | | Ramp1 |
| | Rarres2 | Pdgfd | | | Rarres2 |
| | Rassf8 | Pdgfrl | | | Rassf8 |
| | Rbbp9 | Pdlim1 | | | Rbbp9 |
| | Reck | Pdlim5 | | | Reck |
| | Rnd1 | Pgcp | | | Rnd1 |
| | Rock1 | Phtf2 | | | Rock1 |
| | Rybp | Pigm | | | Rybp |
| | S100a4 | Pip5k1a | | | S100a4 |
| | Sacs | Plaur | | | Sacs |
| | Scx | Plekha1 | | | Scx |
| | Sec22l1 | Plod2 | | | Sec22l1 |
| | Sec23a | Pols | | | Sec23a |
| | Sec24a | Psip1 | | | Sec24a |
| | Sema3c | Ptprz1 | | | Sema3c |
| | Serpine1 | Pycr1 | | | Serpine1 |
| | Sfrp2 | Rab23 | | | Sfrp2 |
| | Sgcb | Rab31 | | | Sgcb |
| | Sgce | Rabep1 | | | Sgce |
| | Sgcg | Ramp1 | | | Sgcg |
| | Sh3bgr | Rarres2 | | | Sh3bgr |
| | Six5 | Rassf8 | | | Six5 |
| | Slc7a1 | Rbbp9 | | | Slc7a1 |
| | Slc9a2 | Reck | | | Slc9a2 |
| | Smurf2 | Rnd1 | | | Smurf2 |
| | Srm | Rock1 | | | Srm |
| | Srprb | Rybp | | | Srprb |
| | Stch | S100a4 | | | Stch |
| | Stk25 | Sacs | | | Stk25 |
| | Stk38l | Scx | | | Stk38l |
| | Stt3a | Sec22l1 | | | Stt3a |
| | Sulf1 | Sec23a | | | Sulf1 |
| | Surb7 | Sec24a | | | Surb7 |
| | Sync | Sema3c | | | Sync |
| | Tdrd3 | Serpine1 | | | Tdrd3 |
| | Tgfb2 | Sfrp2 | | | Tgfb2 |
| | Thbs1 | Sgcb | | | Thbs1 |
| | Tmem39a | Sgce | | | Tmem39a |
| | Tmem45a | Sgcg | | | Tmem45a |
| | Tmem68 | Sh3bgr | | | Tmem68 |
| | Tmtc3 | Six5 | | | Tmtc3 |
| | Tnfaip1 | Slc7a1 | | | Tnfaip1 |
| | Tnfrsf11a | Slc9a2 | | | Tnfrsf11a |
| | Tnfrsf12a | Smurf2 | | | Tnfrsf12a |
| | Tnrc15 | Srm | | | Tnrc15 |
| | Tparl | Srprb | | | Tparl |
| | Tshz3 | Stch | | | Tshz3 |
| | Tspan2 | Stk25 | | | Tspan2 |
| | Tspan6 | Stk38l | | | Tspan6 |
| | Unc5d | Stt3a | | | Unc5d |
| | Vkorc1 | Sulf1 | | | Vkorc1 |
| | Wif1 | Surb7 | | | Wif1 |
| | Wisp2 | Sync | | | Wisp2 |
| | Wtip | Tdrd3 | | | Wtip |
| | Yipf5 | Tgfb2 | | | Yipf5 |
| | Zbtb41 | Thbs1 | | | Zbtb41 |
| | Zcchc10 | Tmem39a | | | Zcchc10 |
| | Zfp148 | Tmem45a | | | Zfp148 |
| | Zfp451 | Tmem68 | | | Zfp451 |
| | Zfp9 | Tmtc3 | | | Zfp9 |
| | | Tnfaip1 | | | |
| | | Tnfrsf11a | | | |
| | | Tnfrsf12a | | | |
| | | Tnrc15 | | | |
| | | Tparl | | | |
| | | Tshz3 | | | |
| | | Tspan2 | | | |
| | | Tspan6 | | | |
| | | Unc5d | | | |
| | | Vkorc1 | | | |
| | | Wif1 | | | |
| | | Wisp2 | | | |
| | | Wtip | | | |
| | | Yipf5 | | | |
| | | Zbtb41 | | | |
| | | Zcchc10 | | | |
| | | Zfp148 | | | |
| | | Zfp451 | | | |
| | | Zfp9 | | | |

TABLE 14
Numbers of Differentially Expressed Genes with Unique Entrez Gene Symbols
for Diabetic ApoE null mice vs. Non-Diabetic ApoE null mice with Positive log Fold
Change, and for Diabetic ApoE null/RAGE null vs. Diabetic ApoE null with Negative log
Fold Change and their Boolean Subsets

| | | | | Diabetic ApoE | |
|---|---|---|---|---|---|
| | | | Diabetic ApoE null | null mice vs. | |
| | | | mice vs. Non-Diabetic | Non-Diabetic | Diabetic ApoE null/ |
| Diabetic | | | ApoE null mice | ApoE null mice | RAGE null vs. |
| ApoE null | | | Positive | Positive NOT | Diabetic ApoE null |
| mice vs. | Diabetic ApoE | Diabetic ApoE null mice | INTERSECTION | Diabetic ApoE | mice Negative NOT |
| Non-Diabetic | null/RAGE null | vs. Non-Diabetic ApoE null | Diabetic ApoE null/ | null/RAGE null | Diabetic ApoE null |
| ApoE null | vs. Diabetic | mice Positive UNION | RAGE null vs. Diabetic | vs. Diabetic | mice vs. Non-Diabetic |
| mice | ApoE null mice | Diabetic ApoE null/RAGE null vs. | ApoE null mice | ApoE null mice | ApoE null mice |
| Positive | Negative | Diabetic ApoE null mice Negative | Negative | Negative | Positive |
| Anxa3 | Acbd3 | Anxa3 | Chordc1 | Anxa3 | Acbd3 |
| Chordc1 | Anapc1 | Chordc1 | Crem | Ctps | Anapc1 |
| Crem | Anln | Crem | Cyp26b1 | Eef1e1 | Anln |
| Ctps | Arf3 | Ctps | Dnaja1 | Emp1 | Arf3 |
| Cyp26b1 | Arfip1 | Cyp26b1 | Golt1b | Figf | Arfip1 |
| Dnaja1 | Armc8 | Dnaja1 | Hsp110 | Fn1 | Armc8 |
| Eef1e1 | Armcx3 | Eef1e1 | Hspa1b | Hexim1 | Armcx3 |
| Emp1 | B3galnt2 | Emp1 | Rybp | Ifitm2 | B3gaint2 |
| Figf | Bcl6 | Figf | S100a4 | Kpna2 | Bcl6 |
| Fn1 | Bdh1 | Fn1 | Slc7a1 | Kras | Bdh1 |
| Golt1b | Bnip2 | Golt1b | Thbs1 | Lmnb2 | Bnip2 |
| Hexim1 | Btaf1 | Hexim1 | Tparl | Mtmr4 | Btaf1 |
| Hsp110 | Bxdc2 | Hsp110 | Tshz3 | Pvt1 | Bxdc2 |
| Hspa1b | Bysl | Hspa1b | Tspan6 | Rbm8a | Bysl |
| Ifitm2 | C1galt1 | Ifitm2 | | Reln | C1galt1 |
| Kpna2 | C1qtnf2 | Kpna2 | | Sfpq | C1qtnf2 |
| Kras | Cacna2d1 | Kras | | Sfrs3 | Cacna2d1 |
| Lmnb2 | Cav3 | Lmnb2 | | Tm4sf1 | Cav3 |
| Mtmr4 | Cbx5 | Mtmr4 | | Ube1l2 | Cbx5 |
| Pvt1 | Cdr2 | Pvt1 | | Wwc2 | Cdr2 |
| Rbm8a | Chordc1 | Rbm8a | | | Chma1 |
| Reln | Chrna1 | Reln | | | Chsy1 |
| Rybp | Chsy1 | Rybp | | | Ckap4 |
| S100a4 | Ckap4 | S100a4 | | | Cnn3 |
| Sfpq | Cnn3 | Sfpq | | | Col5a2 |
| Sfrs3 | Col5a2 | Sfrs3 | | | Copz2 |
| Slc7a1 | Copz2 | Slc7a1 | | | Creb3l2 |
| Thbs1 | Creb3l2 | Thbs1 | | | Crispld1 |
| Tm4sf1 | Crem | Tm4sf1 | | | Cthrc1 |
| Tparl | Crispld1 | Tparl | | | Dap |
| Tshz3 | Cthrc1 | Tshz3 | | | Dapk2 |
| Tspan6 | Cyp26b1 | Tspan6 | | | Dgkg |
| Ube1l2 | Dap | Ube1l2 | | | Dock7 |
| Wwc2 | Dapk2 | Wwc2 | | | Dscr1l1 |
| | Dgkg | Acbd3 | | | Dusp12 |
| | Dnaja1 | Anapc1 | | | Dynll1 |
| | Dock7 | Anln | | | Efhc2 |
| | Dscr1l1 | Arf3 | | | Eln |
| | Dusp12 | Arfip1 | | | Emb |
| | Dynll1 | Armc8 | | | Emp2 |
| | Efhc2 | Armcx3 | | | Emp3 |
| | Eln | B3galnt2 | | | Enah |
| | Emb | Bcl6 | | | Endod1 |
| | Emp2 | Bdh1 | | | Ereg |
| | Emp3 | Bnip2 | | | Ero1l |
| | Enah | Btaf1 | | | Ext1 |
| | Endod1 | Bxdc2 | | | Fgf1 |
| | Ereg | Bysl | | | Fjx1 |
| | Ero1l | C1galt1 | | | Fkbp14 |
| | Ext1 | C1gtnf2 | | | Fryl |
| | Fgf1 | Cacna2d1 | | | Gal3st4 |
| | Fjx1 | Cav3 | | | Ggh |
| | Fkbp14 | Cbx5 | | | Gm347 |
| | Fryl | Cdr2 | | | Gm70 |
| | Gal3st4 | Chrna1 | | | Gmds |
| | Ggh | Chsy1 | | | Gne |
| | Gm347 | Ckap4 | | | Gpr125 |
| | Gm70 | Cnn3 | | | Gpr177 |
| | Gmds | Col5a2 | | | Hbegf |
| | Gne | Copz2 | | | Hcfc2 |
| | Golt1b | Creb3l2 | | | Hdac9 |
| | Gpr125 | Crispld1 | | | Hdlbp |
| | Gpr177 | Cthrc1 | | | Hisppd2a |
| | Hbegf | Dap | | | Hrasls |
| | Hcfc2 | Dapk2 | | | Hrb |
| | Hdac9 | Dgkg | | | Hspa1a |
| | Hdlbp | Dock7 | | | Hspb2 |
| | Hisppd2a | Dscr1l1 | | | Htra1 |
| | Hrasls | Dusp12 | | | Il17b |
| | Hrb | Dynll1 | | | Itga9 |
| | Hsp110 | Efhc2 | | | Itgb1bp1 |
| | Hspa1a | Eln | | | Itpr1 |
| | Hspa1b | Emb | | | Kcnab1 |
| | Hspb2 | Emp2 | | | Kdelr2 |
| | Htra1 | Emp3 | | | Kdelr3 |
| | Il17b | Enah | | | Kpna1 |
| | Itga9 | Endod1 | | | Ky |
| | Itgb1bp1 | Ereg | | | Lancl3 |
| | Itpr1 | Ero1l | | | Lasp1 |
| | Kcnab1 | Ext1 | | | Lats2 |
| | Kdelr2 | Fgf1 | | | Lbh |
| | Kdelr3 | Fjx1 | | | Leprel1 |
| | Kpna1 | Fkbp14 | | | Lox |
| | Ky | Fryl | | | Lpp |
| | Lancl3 | Gal3st4 | | | Lrrc61 |
| | Lasp1 | Ggh | | | Lrrfip1 |
| | Lats2 | Gm347 | | | Ltbp1 |
| | Lbh | Gm70 | | | Luzp1 |
| | Leprel1 | Gmds | | | Maged2 |
| | Lox | Gne | | | Mapk6 |
| | Lpp | Gpr125 | | | Mfap5 |
| | Lrrc61 | Gpr177 | | | Mgat5 |
| | Lrrfip1 | Hbegf | | | Mobk1b |
| | Ltbp1 | Hcfc2 | | | Mtdh |
| | Luzp1 | Hdac9 | | | Myh10 |
| | Maged2 | Hdlbp | | | Ndel1 |
| | Mapk6 | Hisppd2a | | | Nedd9 |
| | Mfap5 | Hrasls | | | Nhlrc2 |
| | Mgat5 | Hrb | | | Nrk |
| | Mobk1b | Hspa1a | | | Ntf3 |
| | Mtdh | Hspb2 | | | Olfml2b |
| | Myh10 | Htra1 | | | P4ha1 |
| | Ndel1 | Il17b | | | Pank2 |
| | Nedd9 | Itga9 | | | Pcgf5 |
| | Nhlrc2 | Itgb1bp1 | | | Pdgfd |
| | Nrk | Itpr1 | | | Pdgfrl |
| | Ntf3 | Kcnab1 | | | Pdlim1 |
| | Olfml2b | Kdelr2 | | | Pdlim5 |
| | P4ha1 | Kdelr3 | | | Pgcp |
| | Pank2 | Kpna1 | | | Phtf2 |
| | Pcgf5 | Ky | | | Pigm |
| | Pdgfd | Lancl3 | | | Pip5k1a |
| | Pdgfrl | Lasp1 | | | Plaur |
| | Pdlim1 | Lats2 | | | Plekha1 |
| | Pdlim5 | Lbh | | | Plod2 |
| | Pgcp | Leprel1 | | | Pols |
| | Phtf2 | Lox | | | Psip1 |
| | Pigm | Lpp | | | Ptprz1 |
| | Pip5k1a | Lrrc61 | | | Pycr1 |
| | Plaur | Lrrfip1 | | | Rab23 |
| | Plekha1 | Ltbp1 | | | Rab31 |
| | Plod2 | Luzp1 | | | Rabep1 |
| | Pols | Maged2 | | | Ramp1 |
| | Psip1 | Mapk6 | | | Rarres2 |
| | Ptprz1 | Mfap5 | | | Rassf8 |
| | Pycr1 | Mgat5 | | | Rbbp9 |
| | Rab23 | Mobk1b | | | Reck |
| | Rab31 | Mtdh | | | Rnd1 |
| | Rabep1 | Myh10 | | | Rock1 |
| | Ramp1 | Ndel1 | | | Sacs |
| | Rarres2 | Nedd9 | | | Scx |
| | Rassf8 | Nhlrc2 | | | Sec22l1 |
| | Rbbp9 | Nrk | | | Sec23a |
| | Reck | Ntf3 | | | Sec24a |
| | Rnd1 | Olfml2b | | | Sema3c |
| | Rock1 | P4ha1 | | | Serpine1 |
| | Rybp | Pank2 | | | Sfrp2 |
| | S100a4 | Pcgt5 | | | Sgcb |
| | Sacs | Pdgfd | | | Sgce |
| | Scx | Pdgfrl | | | Sgcg |
| | Sec22l1 | Pdlim1 | | | Sh3bgr |
| | Sec23a | Pdlim5 | | | Six5 |
| | Sec24a | Pgcp | | | Slc9a2 |
| | Sema3c | Phtf2 | | | Smurf2 |
| | Serpine1 | Pigm | | | Srm |
| | Sfrp2 | Pip5k1a | | | Srprb |
| | Sgcb | Plaur | | | Stch |
| | Sgce | Plekha1 | | | Stk25 |
| | Sgcg | Plod2 | | | Stk38l |
| | Sh3bgr | Pols | | | Stt3a |
| | Six5 | Psip1 | | | Sulf1 |
| | Slc7a1 | Ptprz1 | | | Surb7 |
| | Slc9a2 | Pycr1 | | | Sync |
| | Smurf2 | Rab23 | | | Tdrd3 |
| | Srm | Rab31 | | | Tgfb2 |
| | Srprb | Rabep1 | | | Tmem39a |
| | Stch | Ramp1 | | | Tmem45a |
| | Stk25 | Rarres2 | | | Tmem68 |
| | Stk38l | Rassf8 | | | Tmtc3 |
| | Stt3a | Rbbp9 | | | Tnfaip1 |
| | Sulf1 | Reck | | | Tnfrsf11a |
| | Surb7 | Rnd1 | | | Tnfrsf12a |
| | Sync | Rock1 | | | Tnrc15 |
| | Tdrd3 | Sacs | | | Tspan2 |
| | Tgfb2 | Scx | | | Unc5d |
| | Thbs1 | Sec22l1 | | | Vkorc1 |
| | Tmem39a | Sec23a | | | Wif1 |
| | Tmem45a | Sec24a | | | Wisp2 |
| | Tmem68 | Sema3c | | | Wtip |
| | Tmtc3 | Serpine1 | | | Yipf5 |
| | Tnfaip1 | Sfrp2 | | | Zbtb41 |
| | Tnfrsf11a | Sgcb | | | Zcchc10 |
| | Tnfrsf12a | Sgce | | | Zfp148 |
| | Tnrc15 | Sgcg | | | Zfp451 |
| | Tparl | Sh3bgr | | | Zfp9 |
| | Tshz3 | Six5 | | | |
| | Tspan2 | Slc9a2 | | | |
| | Tspan6 | Smurf2 | | | |
| | Unc5d | Srm | | | |
| | Vkorc1 | Srprb | | | |
| | Wif1 | Stch | | | |
| | Wisp2 | Stk25 | | | |
| | Wtip | Stk38l | | | |
| | Yipf5 | Stt3a | | | |
| | Zbtb41 | Sulf1 | | | |
| | Zcchc10 | Surb7 | | | |
| | Zfp148 | Sync | | | |
| | Zfp451 | Tdrd3 | | | |
| | Zfp9 | Tgfb2 | | | |
| | | Tmem39a | | | |
| | | Tmem45a | | | |
| | | Tmem68 | | | |
| | | Tmtc3 | | | |
| | | Tnfaip1 | | | |
| | | Tnfrsf11a | | | |
| | | Tnfrsf12a | | | |
| | | Tnrc15 | | | |
| | | Tspan2 | | | |
| | | Unc5d | | | |
| | | Vkorc1 | | | |
| | | Wif1 | | | |
| | | Wisp2 | | | |
| | | Wtip | | | |
| | | Yipf5 | | | |
| | | Zbtb41 | | | |
| | | Zcchc10 | | | |
| | | Zfp148 | | | |
| | | Zfp451 | | | |
| | | Zfp9 | | | |

TABLE 15
Numbers of Differentially Expressed Genes with Unique Entrez Gene
Symbols for Diabetic ApoE null vs. Non-Diabetic ApoE null mice with
Negative log Fold Change, and for Diabetic ApoE null/RAGE null vs. Diabetic
ApoE null mice with Positive log Fold Change and their Boolean Subsets

| | | | | Diabetic ApoE null | |
|---|---|---|---|---|---|
| | | | Diabetic ApoE null mice | mice vs. Non-Diabetic | |
| | Diabetic ApoE | | vs. Non-Diabetic ApoE | ApoE null mice | Diabetic ApoE null/RAGE |
| Diabetic ApoE | null/RAGE | Diabetic ApoE null | null mice Negative | Negative NOT | null vs. Diabetic ApoE null |
| null mice vs. | null vs. | mice vs. Non-Diabetic | INTERSECTION | Diabetic ApoE null/ | mice Positive NOT |
| Non-Diabetic | Diabetic ApoE | ApoE null mice Negative UNION | Diabetic ApoE null/ | RAGE null vs. Diabetic | Diabetic ApoE null mice |
| ApoE null mice | null mice | Diabetic ApoE null/RAGE null vs. | RAGE null vs. Diabetic | ApoE null mice | vs. Non-Diabetic ApoE null |
| Negative | Positive | Diabetic ApoE null mice Positive | ApoE null mice Positive | Positive | mice Negative |
| Aacs | Acot1 | Aacs | Erdr1 | Aacs | Acot1 |
| Acaca | Atp6v1d | Acaca | | Acaca | Atp6v1d |
| Acacb | Bcas3 | Acacb | | Acacb | Bcas3 |
| Acly | Ccrn4l | Acly | | Acly | Ccrn4l |
| Acss2 | Chd1 | Acss2 | | Acss2 | Chd1 |
| Agbl3 | Chd2 | Agbl3 | | Agbl3 | Chd2 |
| Erdr1 | Eml5 | Erdr1 | | Fasn | Eml5 |
| Fasn | Erdr1 | Fasn | | Fmo5 | Glo1 |
| Fmo5 | Glo1 | Fmo5 | | H2-Q10 | Glp1r |
| H2-Q10 | Glp1r | H2-Q10 | | Mod1 | Gpatc2 |
| Mod1 | Gpatc2 | Mod1 | | Mtmr11 | Grina |
| Mtmr11 | Grina | Mtmr11 | | Nudt18 | H6pd |
| Nudt18 | H6pd | Nudt18 | | Ppp2r1b | Mrpl15 |
| Ppp2r1b | Mrpl15 | Ppp2r1b | | Pygl | Ngp |
| Pygl | Ngp | Pygl | | Slc1a5 | Pbef1 |
| Slc1a5 | Pbef1 | Slc1a5 | | Slc25a1 | Prlr |
| Slc25a1 | Prlr | Slc25a1 | | Thrsp | Prodh |
| Thrsp | Prodh | Thrsp | | Tkt | Sgol2 |
| Tkt | Sgol2 | Tkt | | | Sh3gl2 |
| | Sh3gl2 | Acot1 | | | Sorbsl |
| | Sorbs1 | Atp6v1d | | | Sord |
| | Sord | Bcas3 | | | St8sia6 |
| | St8sia6 | Ccrn4l | | | Timp4 |
| | Timp4 | Chd1 | | | Trp53inp2 |
| | Trp53inp2 | Chd2 | | | Ube2i |
| | Ube2i | Eml5 | | | Xdh |
| | Xdh | Glo1 | | | |
| | | Glp1r | | | |
| | | Gpatc2 | | | |
| | | Grina | | | |
| | | H6pd | | | |
| | | Mrpl15 | | | |
| | | Ngp | | | |
| | | Pbef1 | | | |
| | | Prlr | | | |
| | | Prodh | | | |
| | | Sgol2 | | | |
| | | Sh3gl2 | | | |
| | | Sorbs1 | | | |
| | | Sord | | | |
| | | St8sia6 | | | |
| | | Timp4 | | | |
| | | Trp53inp2 | | | |
| | | Ube2i | | | |
| | | Xdh | | | |

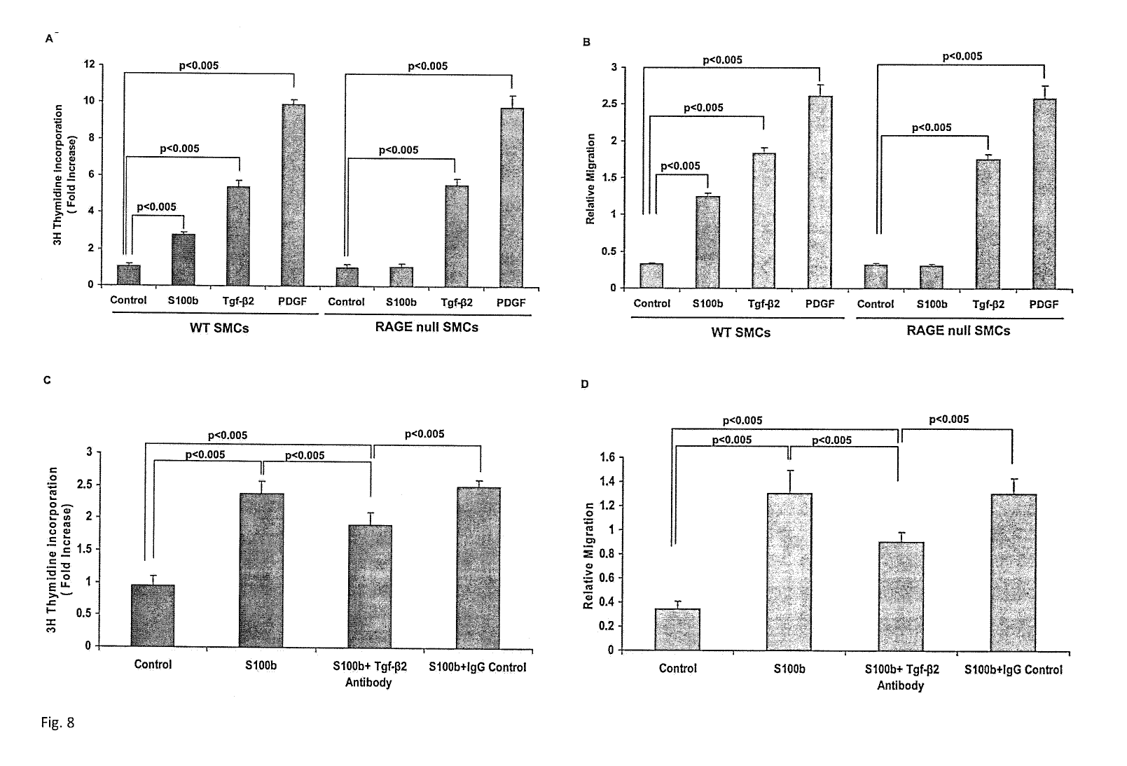

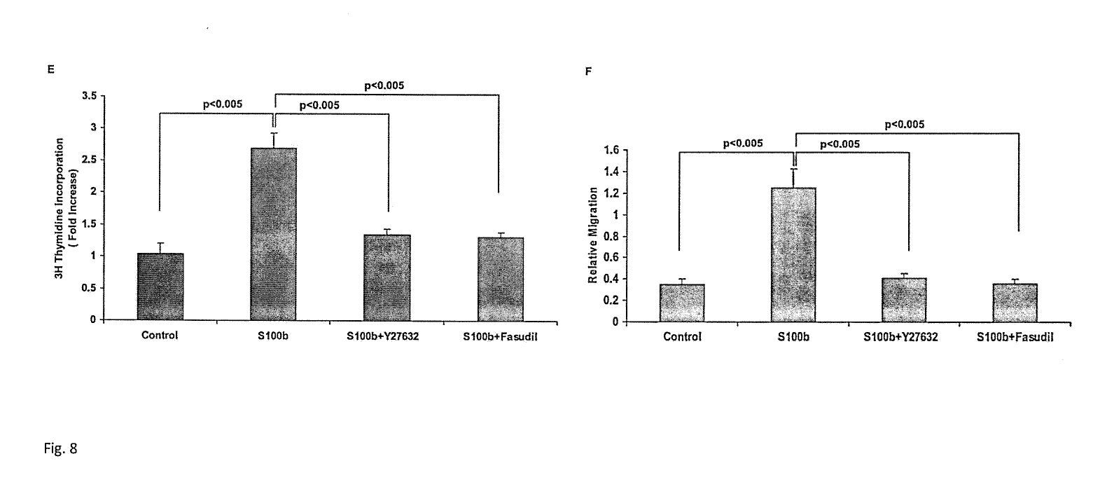

Next, to specifically link RAGE to SMC proliferation and migration, studies were performed in primary SMCs retrieved from RAGE-expressing or RAGE-deficient mouse aortas. As illustrated in FIGS. 8A and 8B, incubation of wild-type SMCs with RAGE ligand S100B resulted in significantly increased proliferation and migration, but S100B failed to stimulate proliferation and migration in RAGE-deficient SMCs. These data indicate that RAGE is required for the actions of S100B on SMC proliferation and migration. Note that in wild-type and RAGE-deficient SMCs, incubation with Tgf-β2 or a non-RAGE ligand PDGF increased proliferation and migration, suggesting that Tgf-β2 and PDGF are not direct ligands of RAGE and that exogenous addition of Tgf-β2 to RAGE-deficient cells restores proliferation and migration responses (FIGS. 8A and 8B, respectively).

Next, it was necessary to establish that RAGE ligand-stimulated SMC proliferation and migration required Tgf-β2 and ROCK1 action. We treated wild-type SMCs with S100B in the presence or absence of Tgf-β2 or ROCK1 inhibitors. Consistent with key roles for Tgf-β2 in S100B-mediated effects on SMCs, pre-treatment of wild-type SMCs with anti-Tgf-β2 antibody resulted in a significant decrease in proliferation and migration compared to treatment with an IgG control (FIGS. 8C & 8D, respectively). ROCK signaling was implicated in the S100B modulation of SMC properties, as treatment of SMCs with S100B in the presence of ROCK inhibitors Y27632 or fasudil significantly reduced S100B-stimulated proliferation and migration (FIGS. 8E & 8F, respectively).

### Discussion

These findings illustrate the mechanisms by which diabetes accelerates atherogenesis in ApoE null mice, and by which RAGE deletion slows atherogenesis in diabetic ApoE null mice. The effect of diabetes on ApoE null mice was analyzed. The implications of the Pathway Express analysis (FIGS. 1 and 2, Tables 7 and 8) are as follows: diabetes up-regulates Thbs1; no change in levels of LTBP1 was detected for this comparison; the amount of activated Tgf-β2 may increase because the total amount of Tgf-β2 increases, and because of increased activation due to up-regulation of Thbs1; since Tgf-β2 activates Tgf-βR1&2 complex (TGFBR) and since the amount of activated TGF-β2 increases, the amount of activated TGFBR increases; since no change in the amount of SMURF2 mRNA was detected in this comparison, interaction with SMURF2, which targets TGFBR1 for destruction (28), will not change the amount of total or activated Tgf-βR; since Tgf-βR complex indirectly activates RhoA, by a mechanism that has not yet been fully characterized (29-30), and since the amount of activated Tgf-βR complex increases, the amount of activated RhoA increases; since RhoA activates ROCK1 (31-32), and since the amount of activated RhoA increases, the amount of activated ROCK1 increases; since ROCK1 accelerates atherogenesis (ATHRG), and since the extent of ROCK1 activation increases, acceleration of atherosclerosis ensues.

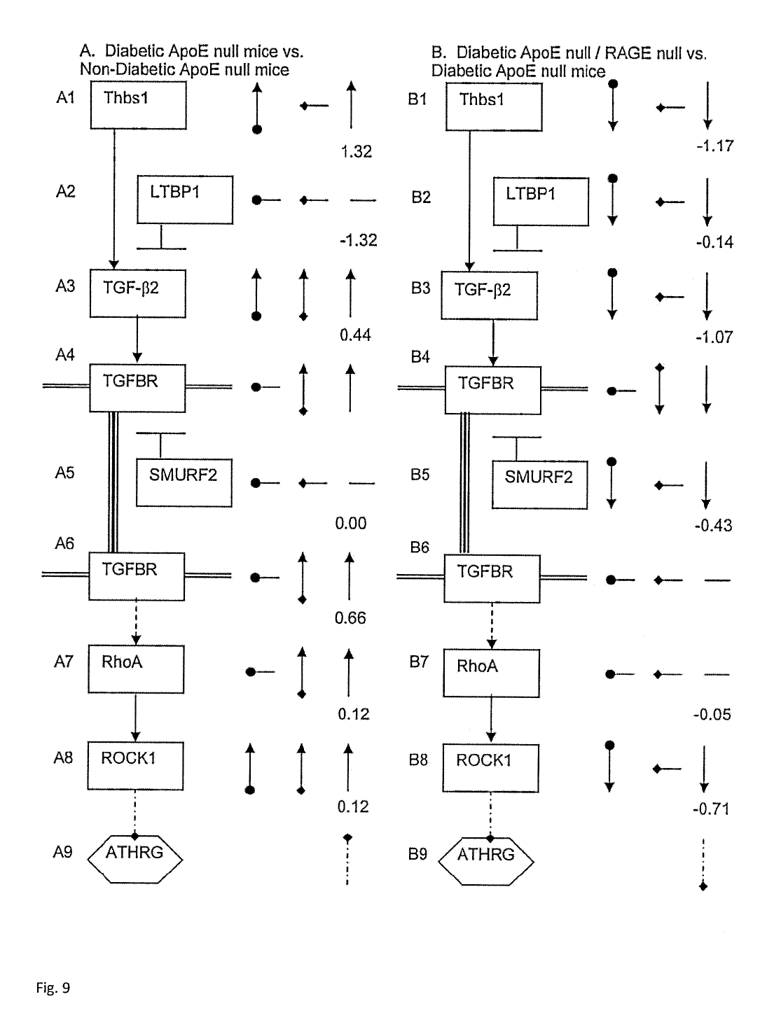

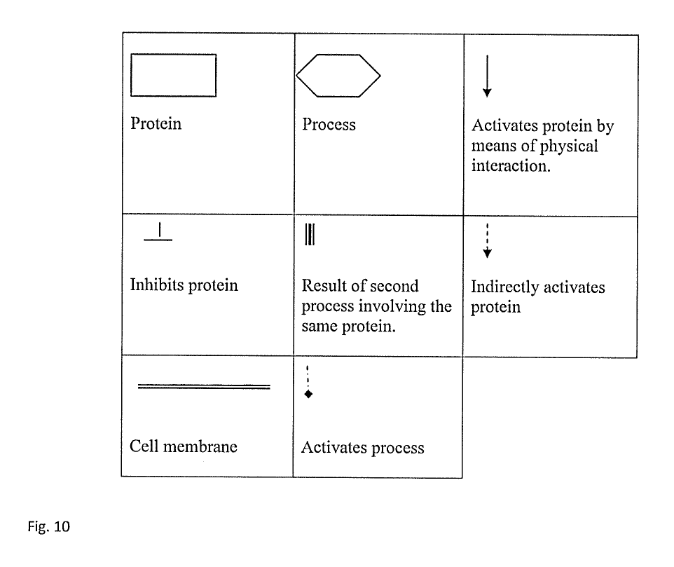

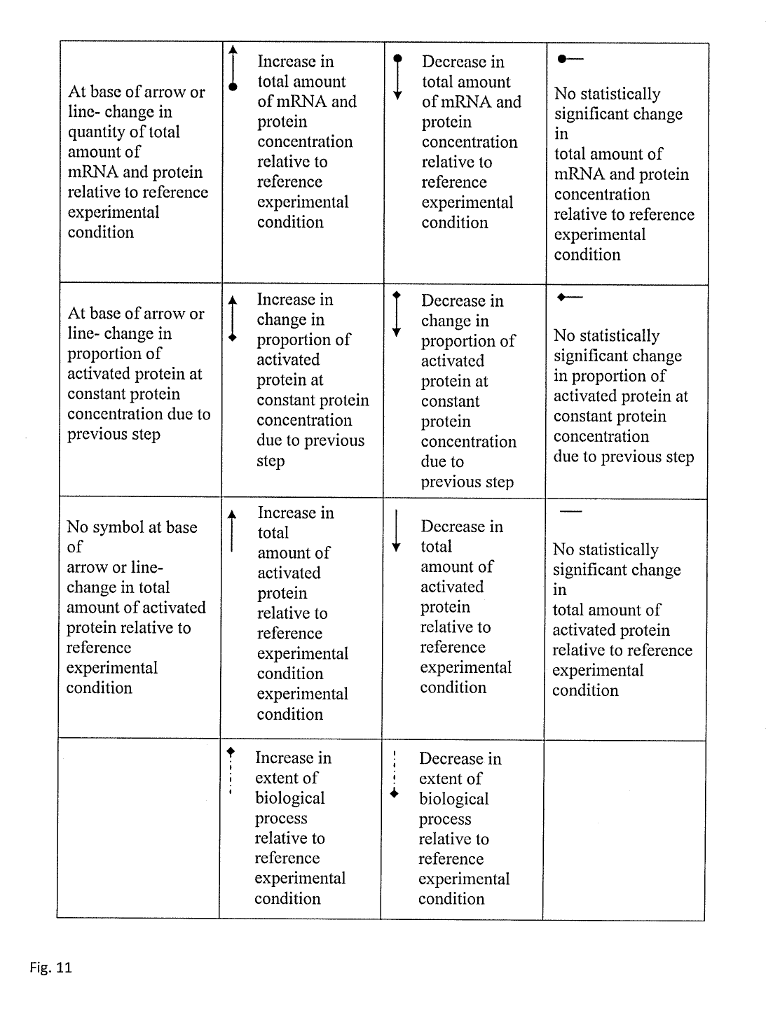

The mechanism by which RAGE deletion delays acceleration of atherosclerosis in diabetic ApoE null mice was also analyzed. Diabetic ApoE null/RAGE null vs. diabetic ApoE null mice: the amount of Thbs1 decreases upon deletion of RAGE; LTBP1 expression decreases; the amount of activated TGF-β2 decreases as the total amount of TGF-β2 decreases. (The proportion of activated TGF-β2 decreases because of the decrease in Thbs1, however, this effect may be cancelled, all or in part, by the decrease in TGF-β2 deactivation by LTBP1 accompanying the decrease in LTBP1); since TGF-β2 activates Tgf-βR , and since the amount of activated TGF-β2 decreases, the amount of activated complex decreases; the amount of SMURF2 decreases; since SMURF2 deactivates TGFBR1, by targeting it for destruction, and the amount of SMURF2 decreases, the amount of Tgf-βR1 may increase, canceling all or part of the effect of decrease in activated Tgf-βR; since Tgf-βR complex indirectly activates RhoA, by a mechanism that has not yet been fully characterized, and since the amount of activated Tgf-βR is approximately unchanged, the amount of activated ROCK1 is also approximately unchanged; the total amount of ROCK1 decreases (since the amount of activated RhoA remains roughly constant, and the total amount of ROCK1 decreases, the amount of activated ROCK1 decreases); since ROCK1 accelerates atherosclerosis (ATHRG) (18-20), and since the amount of activated ROCK1 decreases, atherosclerosis is reduced. See FIGS. 9-11. In summary, The observed reduction of accelerated atherosclerosis in diabetic ApoE null/RAGE null vs. diabetic ApoE null mice occurs, all or in part, through the ROCK1 branch of the TGF-β pathway.

### REFERENCES

1. Park L, Raman K G, Lee K J, Lu Y, Ferran L J, Chow W S, Stern D, Schmidt A M. Suppression of accelerated diabetic atherosclerosis by the soluble receptor for advanced glycation endproducts. Nat Med 1998; 4: 1025-1031.
2. Kislinger T, Tanji N, Wendt T, Qu W, Lu Y, Ferran L J Jr, Taguchi A, Olson K, Bucciarelli L, Goova M, Hofmann M A, Cataldegirmen G, D'Agati V, Pischetsrieder M, Stern D M, Schmidt A M. Receptor for advanced glycation endproducts mediates inflammation and enhanced expression of tissue factor in vasculature of diabetic apolipoprotein E null mice. Arterioscler Thromb Vasc Biol 2001; 21: 905-910
3. Bucciarelli L G, Wendt T, Qu W, Lu Y, Lalla E, Rong L L, Goova M T, Moser B, Kislinger T, Lee D C, Kashypa Y, Stern D M, Schmidt A M. RAGE blockade stabilizes established atherosclerosis in diabetic apolipoprotein E null mice. Circ 2002; 106: 2827-2835.
4. Wendt T, Harja E, Bucciarelli L, Qu W, Lu Y, Rong L L, Jenkins D G, Stein G, Schmidt A M, Yan S F. RAGE modulates vascular inflammation and atherosclerosis in a murine model of type 2 diabetes. Atherosclerosis 2006; 185: 70-77
5. Harja E, Bu D X, Hudson B I, Chang J S, Shen X, Hallam K, Kalea A Z, Lu Y, Rosario R H, Oruganti S, Nikolla Z, Belay D, Lalla E, Ramasamy R, Yan S F, Schmidt A M. Vascular and inflammatory stresses mediate atherosclerosis via RAGE and its ligands in apoE−/−mice. J Clin Invest 2008; 118: 183-194.
6. Soro-Paavonen A, Watson A M, Li J, Paavonen K, Koitka A, Calkin A C, Barit D, Coughlan M T, Drew B G, Lancaster G I, Thomas M, Forbes J M, Nawroth P P, Bierhaus A, Cooper M E, Jandeleit-Dahm K A. Receptor for advanced glycation endproducts (RAGE) deficiency attenuates the development of atherosclerosis in diabetes. Diabetes 2008; 57: 2461-2469
7. Kimura K, Ito M, Amano K, Chihara Y, Fukata M, Nakafuku M, Yamamori B, Feng J, Nakano T, Okawa K, Iwamatsu A, Kaibuchi K. (1996) Regulation of myosin phosphatase by Rho and Rho-associated kinase (Rho-kinase). Science 273:245-248.
8. Mong P Y, Petrulio C, Kaufman H L, Wang Q Activation of Rho kinase by TNF-alpha is required for JNK activation in human pulmonary microvascular endothelial cells. J Immunol 2008; 180:550-558.
9. Libby P. Inflammation in atherosclerosis. Nature 2002; 420, 868-874
10. Grainger D J. Transforming growth factor beta and atherosclerosis: so far, so good for the protective cytokine hypothesis. Arterioscler Thromb Vasc Biol 2004; 24: 399-404
11. Grainger D J. TGF-beta and atherosclerosis in man. Cardiovasc Res 2007; 74: 213-222
12. Mallat Z, Tedgui A. The role of transforming growth factor beta in atherosclerosis: novel insights and future perspectives. Curr Opin Lipidol 2002; 13: 523-529
13. Grainiger D J, Mosedale D E, Metcalfe J C, Bottinger E P. Dietary fat and reduced levels of TGF beta 1 act synergistically to promote activation of the vascular endothelium and formation of lipid lesions. J Cell Sci 2000; 113: 2355-2361
14. Gojova A, Brun V. Esposito B, Cottrez F, Gourdy P, Ardouin P, Tedgui A, Mallat Z, Groux H. Specific abrogation of transforming growth factor-beta signaling in T cells alters atherosclerotic lesion size and composition in mice. Blood 2003; 102: 4052-4058
15. Mallat Z, Gojova A, Marchiol-Fournigault C, Esposito B, Kamate C, Merval R, Fradelizi D, Tedgui A. Inhibition of transforming growth factor-beta signaling accelerates atherosclerosis and induces an unstable plaque phenotype in mice. Circ Res 2001; 89: 930-934
16. Draghici S, Khatri P, Tarca A L, Amin K, Done A, Voichita C, Georgescu C, Romero R. A systems biology approach for pathway level analysis. Genome Res 2007; 17:1537-1545.
17. Miyazono K, Hellman U, Hellman C, Wernstedt C, Heldin C H. Latent high molecular weight complex of transforming growth factor beta 1. Purification from human platelets and structural characterization J Biol Chem 1998; 263: 6407-6415
18. Saharinen J, Hyytiainen M, Taipale J, Keski-Oja J. Latent transforming growth factor beta binding proteins (LTBPs)-structural extracellular matrix proteins for targeting TGF beta action. Cytokine Growth Factor Rev 1999; 10: 99-117
19. Saharinen J, Taipale J, Keski-Oja J. Association of the small latent transforming growth factor-beta with an eight cysteine repeat of its binding protein LTBP-1. EMBO J. 1996; 15: 245-253
20. Mallat Z, Gojova A, Sauzeau V. Brun V, Silvestre J S, Esposito B, Merval R, Groux H, Loirand G, Tedgui A. Rho-associated protein kinase contributes to early atherosclerotic lesion formation in mice. Circ Res 2003; 93: 884-888
21. Wang H W, Liu P Y, Oyama N, Rikitake Y, Kitamoto S, Gitlin J, Liao J K, Boisvert W A. Deficiency of ROCK1 in bone marrow-derived cells protects against atherosclerosis in LDL receptor null mice. FASEB J 2008; 22: 3561-3570
22. Noma K, Rikitake Y, Oyama N, Yan G, Alcaide P, Liu P Y, Wang H, Ahl D, Sawada N, Okamoto R, Hiroi Y, Shimizu K, Luscinskas F W, Sun J, Liao J K. ROCK1 mediates leukocyte recruitment and neointima formation following vascular injury. J Clin Invest 2008; 118: 1632-1644
23. Riessen R, Kearney M, Lawler J, Isner J M. Immunolocalization of thrombospondin-1 in human atherosclerotic and restenotic arteries. Am Heart J 1998; 135: 357-364
24. Lane T F, Iruela-Arispe M L, Sage E H. Regulation of gene expression by SPARC during angiogenesis in vitro. Changes in fibronectin, thrombospondin-1, and plasminogen activator inhibitor-1. J Biol Chem (1992) 267: 16736-16745
25. Shimokawa H, Rashid M. Development of Rho kinase inhibitors for cardiovascular medicine. Trends Pharmacol Sci 2007; 28: 296-302
26. Kaysak P, Rasmussen R K, Causing C G, Bonni S, Zhu H, Thomsen G H, Wrana J L. Smad7 binds to Smurf2 to form E3 ubiquitin ligase that targets the TGF beta receptor for degradation. Mol Cell 2000; 6: 1365-1375
27. Clements Rt, Minnear F L, Singer H A, Keller R S, Vincent P A. Rho A and Rho-kinase dependent and independent signals mediate TGF-beta induced pulmonary endothelial cytoskeletal reorganization and permeability. Am J Physiol Cell Mol Physiol 2005; 288: L294-L306
28. Rosman D S, Phukan S, Huang C C, Pasche B. TGFBR1*6A enhances the migration and invasion of MCF-7 breast cancer cells through RhoA activation. Cancer Res 2008; 68: 1319-1328
29. Fujisawa K, Fujita A, Ishizaki T, Saito Y, Narumiya S. Identification of the Rho-binding domain of p160ROCK, a Rho-associated coiled-coil containing protein kinase. J Biol Chem 1996; 271: 23022-23028
30. Ishizaki T, Mackawa M, Fujisawa K, Okawa K, Iwamatsu A, Fujita A, Watanabe N, Saito Y, Kakizuka A, Morii N, Narumiya S. The small GTP-binding protein Rho binds to and activates a 160 kDa Ser/Thr protein kinase homologous to myotonic dystrophy kinase. EMBO J. 1996; 15: 1885-1893
31. Ruiz-Ortega M, Rodriguez Vita J, Sanchez Lopez E, Carvajal G, Egido J. TGF beta signaling in vascular fibrosis. Cardiovasc Res 2007; 74: 196-206
32. Zegarska J, Paczek L, Pawlowska M, Wycalkowsk A, Michalska W, Ziolkowski J, Gorski A, Rowinski W, Kosieradzki M, Kwiatkowski A, Gornicka B, Ziarkiewicz-Wroblewska B. Increased mRNA expression of transforming growth factor beta in the arterial wall of chronically rejected renal allografts in humans. Transplant Proc 2006; 38: 115-11B
33. Ryan S T, Koteliansky V E, Gotwals P J, Lindner V. Transforming growth factor-beta dependent events in vascular remodeling following arterial injury. J Vasc Res 2003; 40: 37-46
34. Sun L, Ishida T, Yasuda T, Kojima Y, Honjo T, Yamamoto Y, Yamamoto H, Ishibashi S, Hirata K, Hayashi Y. RAGE mediates oxidized LDL-induced pro-inflammatory effects and atherosclerosis in non-diabetic LDL receptor-deficient mice. Cardiovasc Res 2009; epub ahead of print
35. Ming X F, Barandier C, Viswambharan H, Kwak B R, Mach F, Mazzolai L, Hayoz D, Ruffieux J, Rusconi S, Montani J P, Yang Z. Thrombin stimulates human endothelial arginase enzymatic activity via RhoA/ROCK pathway: implications for atherosclerotic endothelial dysfunction. Circ 2004; 110: 3708-3714
36. Noma K, Goto C, Nishioka K, Jitsuiki D, Umemura T, Jitsuiki D, Nakagawa K, Oshima T, Chayama K, Yoshizumi M, Higashi Y. Roles of rho-associated kinase and oxidative stress in the pathogenesis of aortic stiffness. J Am Coll Cardiol 2007; 49: 698-705
37. Nohria A, Prsic A, Liu P Y, Okamoto R, Creager M A, Selwyn A, Liao J K, Ganz P. Statins inhibit rho kinase activity in patients with atherosclerosis. Atherosclerosis 2008; in press

1. A method of treating a renal disease in a subject comprising administering to the subject an amount of an antagonist of receptor for advanced glycation end products (RAGE) effective to treat the renal disease in the subject.

2. The method of claim 1, wherein the renal disease is ureteral obstructive kidney disease or renal fibrosis.

3. A method of treating a disease involving apoptosis of cardiomyocytes in a subject comprising administering to the subject an amount of an antagonist of receptor for advanced glycation end products (RAGE) effective to treat the disease involving apoptosis of cardiomyocytes in the subject.

4. A method of treating a lung disease in a subject comprising administering to the subject an amount of an antagonist of receptor for advanced glycation end products (RAGE) effective to treat the lung disease in the subject.

5. The method of claim 4, wherein the lung disease is Acute Respiratory Distress Syndrome.

6. A method of enhancing the efficacy of a chemotherapeutic agent in inducing apoptosis of a tumor cell in a subject comprising administering to the subject a chemotherapeutic agent and an amount of an antagonist of receptor for advanced glycation end products (RAGE) effective to enhance the efficacy of the chemotherapeutic agent in inducing apoptosis of the tumor cell the subject.

7. The method of claim 6, wherein the tumor cell is a colorectal cancer cell, a brain cancer cell, or a breast cancer cell.

8. The method of claim 6, wherein the chemotherapeutic agent is thymidylate synthase inhibitor BGC9331 or topoisomerase I inhibitor SN-38.

9-17. (canceled)

18. The method of claim 1, wherein the antagonist is a RAGE antibody, a small molecule RAGE antagonist, a fusion protein RAGE antagonist, or a polypeptide RAGE antagonist.

19-62. (canceled)

63. The method of claim 7, wherein the chemotherapeutic agent is thymidylate synthase inhibitor BGC9331 or topoisomerase I inhibitor SN-38.

64. The method of claim 3, wherein the antagonist is a RAGE antibody, a small molecule RAGE antagonist, a fusion protein RAGE antagonist, or a polypeptide RAGE antagonist.

65. The method of claim 4, wherein the antagonist is a RAGE antibody, a small molecule RAGE antagonist, a fusion protein RAGE antagonist, or a polypeptide RAGE antagonist.

66. The method of claim 6, wherein the antagonist is a RAGE antibody, a small molecule RAGE antagonist, a fusion protein RAGE antagonist, or a polypeptide RAGE antagonist.
